(19) United States
(12) Patent Application Publication
(10) Pub. No.: US 2025/0090791 A1
(43) Pub. Date: Mar. 20, 2025

(54) TREATMENT LIGHT CONTROL SYSTEM AND METHOD

(71) Applicant: Koji INOUE, Tokyo (JP)

(72) Inventor: Koji INOUE, Tokyo (JP)

(21) Appl. No.: 18/969,439

(22) Filed: Dec. 5, 2024

Related U.S. Application Data
(63) Continuation of application No. PCT/JP2023/026925, filed on Jul. 24, 2023.

(30) Foreign Application Priority Data
Jul. 27, 2022 (JP) 2022-119478

(51) Int. Cl. A61M 21/00; B05B 1/14

(52) U.S. Cl. CPC A61M 21/00; A61M 2021/0027; A61M 2021/005; A61M 2205/50; B05B 1/14

(57) ABSTRACT

A treatment light control system including: a light source configured to apply blinking light; a controller configured to control a blinking frequency of the light source such that the blinking frequency is included in a predetermined frequency band; and a fine particle discharge apparatus configured to discharge fine particles into a spatial region to which the light source applies light.

### CROSS-REFERENCE TO RELATED APPLICATIONS

This application is a continuation of International Application No. PCT/JP2023/026925, filed Jul. 24, 2023, which claims priority to Japanese Patent Application No. 2022-119478, filed Jul. 27, 2022, the entire contents of each are incorporated herein by reference.

### FIELD

The present invention relates to a treatment light control system and a method.

### BACKGROUND

It is known that diseases pertaining to brain activities such as Alzheimer-type dementia and seasonal affective disorder can be prevented by a light therapy that is continued on a daily basis.

In addition, a glasses-type treatment light application device is known to perform such a light therapy to prevent brain diseases.

In this treatment light application device, LEDs are disposed at a position close to eyes of a user, and the LEDs apply treatment light that blinks at a predetermined frequency, by which a visual stimulus that is preventive of a brain disease can be given to the user.

Since the treatment light application device applies the light from the position close to eyes, the treatment light application device can reliably perform a therapy. However, the light is applied to a user at close range from the device fixed to a face, and thus the user may feel strong discomfort.

At a preventive stage, a therapy recipient generally leads the same life as healthy individuals and does not feel significantly harmful influence from a disease to be prevented. Therefore, there is a case where a stress felt from preventive treatment becomes greater than anxiety for future onset and the preventive treatment is suspended.

That is, a conventional treatment light application device has a trade-off between a therapeutic effect and a continuous ease of use for a user. Thus, there is a demand for development of a system that has a high therapeutic effect and continuous ease of use for a user.

A feature of the present invention is to provide a treatment light control system that has a high therapeutic effect and is easy to continuously use for a user.

### BRIEF DESCRIPTION OF DRAWINGS

FIG. 1A is an external view of a treatment light control system according to a first embodiment of the present invention.

FIG. 1B is an external view of a treatment light control system according to a first embodiment of the present invention.

FIG. 2 is an A-A sectional view of the treatment light control system illustrated in FIG. 1B.

FIG. 3 is a diagram illustrating a flow of data in starting use of the treatment light control system illustrated in FIG. 1A and FIG. 1B.

FIG. 4A is an explanatory diagram about a scene illustrated in FIG. 2.

FIG. 4B is an explanatory diagram about a scene illustrated in FIG. 2.

FIG. 4C is an explanatory diagram about a scene illustrated in FIG. 2.

FIG. 5 is a block diagram of the treatment light control system illustrated in FIG. 1A and FIG. 1B.

FIG. 6A is a diagram illustrating a panel-shape LED light source.

FIG. 6B is a diagram illustrating a panel-shape LED light source.

FIG. 7 is a block diagram illustrating a hardware configuration of a controller illustrated in FIG. 2.

FIG. 8 is a block diagram illustrating a functional configuration of the controller illustrated in FIG. 2.

FIG. 9 is a diagram illustrating pulse waveforms of beams of light that are output at gamma frequencies.

FIG. 10 is a diagram illustrating a pulse waveform of light that is generated by performing PWM control while light is turned on.

FIG. 11 is a diagram of a comparison of pulse waveforms between light amount adjustment by the PWM control and light amount adjustment by only an interval of an ON time.

FIG. 12 is a diagram illustrating an example of a method of generating treatment content.

FIG. 13 is a diagram illustrating a process of inserting light OFF and sound OFF.

FIG. 14 is a diagram illustrating a process of detecting a portion that is not suitable for a therapy from treatment content and correcting the portion.

FIG. 15A is a diagram illustrating a structure of a head unit of a mist generator included in a fine particle discharge device.

FIG. 15B is a diagram illustrating a structure of a head unit of a mist generator included in a fine particle discharge device.

FIG. 16A is a diagram illustrating a structure of a smart speaker of a bath pillow type.

FIG. 16B is a diagram illustrating a structure of a smart speaker of a bath pillow type.

FIG. 17 is a diagram illustrating an example of a display screen of a management application.

FIG. 18 is a diagram illustrating a communication state of data during use of the treatment light control system.

FIG. 19 is a chart illustrating a process in use of the treatment light control system.

FIG. 20 is a diagram illustrating a process of analyzing conditions of a user.

FIG. 21A is a diagram illustrating a modification of a light source.

FIG. 21B is a diagram illustrating a modification of a light source.

FIG. 22 is a diagram illustrating a positional relation in a case where a projector is used as the light source.

FIG. 23 is a diagram illustrating a state where treatment light is projected from the projector onto fine particles.

FIG. 24 is a schematic diagram of AR glasses provided with small projectors.

FIG. 25 is a diagram illustrating a method of generating treatment content according to a modification.

FIG. 26 is a diagram illustrating an example of a visual presentation using fine particles for treatment content.

FIG. 27A is a diagram illustrating a state of the treatment light control system corresponding to an image X in FIG. 26.

FIG. 27B is a diagram illustrating a state of the treatment light control system corresponding to an image X in FIG. 26.

FIG. 28A is a diagram illustrating a state of the treatment light control system corresponding to an image Y in FIG. 26.

FIG. 28B is a diagram illustrating a state of the treatment light control system corresponding to an image Y in FIG. 26.

FIG. 29 is an external view of a case where the treatment light control system is used in a toilet.

FIG. 30 is an external view of a case where the treatment light control system is used in an amusement facility.

FIG. 31 is an external view of a case where the treatment light control system is used in a bedroom.

FIG. 32 is a diagram illustrating a process at a time when the treatment light control system is applied to adjustment of a circadian rhythm.

FIG. 33 is a diagram illustrating an example of a picture that changes in wavelength in chronological order.

FIG. 34 is a diagram illustrating a business model using an inspection system using the treatment light control system.

FIG. 35 is a diagram illustrating a business model using an insurance/financial system using the treatment light control system.

FIG. 36 is a diagram illustrating a business model in which an insurance company provides a platform of stimulus treatment of/inspection for dementia.

FIG. 37 is a diagram illustrating a communication state of data at a time of starting use of the insurance system that uses the treatment light control system.

FIG. 38 is a diagram illustrating a functional configuration of an insurance management server.

FIG. 39 is a diagram illustrating a communication state of data at a time of using the insurance system that uses the treatment light control system.

FIG. 40 is a chart illustrating a process performed by an evaluation module of the management server.

FIG. 41 is a table illustrating an example of a criterion of evaluation pertaining to a primary score that is given in accordance with a frequency of use by a user.

FIG. 42 is a table illustrating an example of a criterion of evaluation pertaining to a weighting coefficient that is given in accordance with a result of inspection.

FIG. 43 is a table illustrating an example of a definition pertaining to details of a reward based on an evaluation score.

FIG. 44 is a chart illustrating a process at a time when a reward is provided to a corporation.

FIG. 45 is a diagram illustrating an example of evaluation of good content.

FIG. 46A is a diagram illustrating examples of periodic changes in illuminance in another modification of the light source.

FIG. 46B is a diagram illustrating examples of periodic changes in illuminance in another modification of the light source.

FIG. 46C is a diagram illustrating examples of periodic changes in illuminance in another modification of the light source.

FIG. 46D is a diagram illustrating examples of periodic changes in illuminance in another modification of the light source.

FIG. 47 is a diagram illustrating a configuration of another modification of the light source.

FIG. 48 is a schematic diagram of experiment conditions of Experiment 1.

FIG. 49 is a diagram illustrating indices of subjective evaluation made by subjects of Experiment 1.

FIG. 50A is an image illustrating appearance of fog under each condition in Experiment 1.

FIG. 50B is an image illustrating appearance of fog under each condition in Experiment 1.

FIG. 50C is an image illustrating appearance of fog under each condition in Experiment 1.

FIG. 50D is an image illustrating appearance of fog under each condition in Experiment 1.

FIG. 51 is a graph illustrating average values of results of the subjective evaluation made by the subjects of Experiment 1 on a condition basis.

FIG. 52 is a schematic diagram of experiment conditions of Experiment 2.

FIG. 53 is a diagram illustrating indices of subjective evaluation made by subjects of Experiment 2.

FIG. 54 is a graph illustrating average values of results of the subjective evaluation made by the subjects of Experiment 2 on a condition basis.

FIG. 55 is a schematic diagram of an example in which a light stimulus device is placed in a bathtub.

FIG. 56 is an external view of an example of placing a liquid discharging device.

FIG. 57 is an external view of an example of placing a liquid discharging device.

FIG. 58 is an external view of a droplet generating device.

### DESCRIPTION OF EMBODIMENTS

An aspect of the present disclosure is a treatment light control system comprising: a light source configured to apply blinking light; a controller configured to control a blinking frequency of the light source such that the blinking frequency is included in a predetermined frequency band; and a fine particle discharge apparatus configured to discharge fine particles into a spatial region to which the light source applies light.

### First Embodiment

Hereinafter, a first embodiment of the present invention will be described in detail with reference to the accompanying drawings.

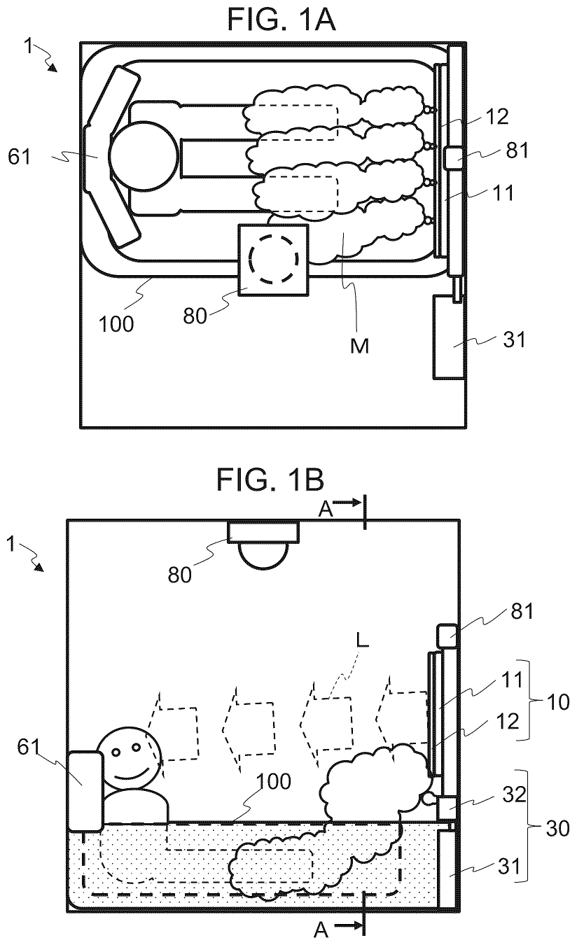

In the drawings for describing the embodiment, the same constituent components will be denoted by the same reference characters in principle, and repetitive descriptions thereof will be omitted. FIG. 1A is an external view of a treatment light control system 1 according to the first embodiment of the present invention. FIG. 1B is an external view of a treatment light control system 1 according to the first embodiment of the present invention.

### (1) Overview of First Embodiment

An overview of the present embodiment will be described.

The treatment light control system 1 according to the present embodiment (hereinafter, simply referred to as a system 1) is a system that has a function of mainly outputting treatment light for performing treatment necessary for a user as a light stimulus acting on eyes of the user.

### (1-1) Overview of Configuration and Usage of System 1

FIG. 1A is a diagram illustrating a configuration of the system 1 according to a typical embodiment (the first embodiment). FIG. 1B is a diagram illustrating a configuration of the system 1 according to a typical embodiment (the first embodiment).

The system 1 according to the first embodiment is used at a time of giving a light stimulus/sound stimulus with a gamma frequency aimed at preventing or improving dementia, or delaying progression of dementia in a bathroom.

In general, in a cerebral cortex in a brain, a group of neurons fire in synchronization with one another to cause synaptic activities, thus bringing about periodic neuronal activities (oscillations). These neuronal activities are categorized by frequency into a theta band (4 to 12 Hz), a gamma band (25 to 100 Hz), and the like. In this description, the gamma frequency denotes frequencies equivalent to those of neuronal activities categorized into the gamma band (25 to 100 Hz). It is considered that neuronal activities in the gamma band are deeply involved particularly in cognitive functions such as attention, memory, and decision making, and it is said that an external input of a stimulus in the gamma band can effectively act on the cognitive functions. In the following description, a band of 25 Hz to 100 Hz is called the gamma band.

As illustrated in FIG. 1A and FIG. 1B, after entering a bathtub 100, the user gives a smart speaker 61 instructions to start treatment and operates devices around the user through a communicating function of the smart speaker 61, thus receiving treatment with the light stimulus and sound stimulus.

The system 1 first turns off an illuminating device 80 in a bathroom and then uses an optical sensor 81 to check that there is no optical noise that influences a therapy. Next, the system 1 applies a light stimulus with the gamma frequency from a light source 11, displays visual content on a liquid crystal display (a visual display unit 12), and discharges mist from a mist generator 32 toward the bathtub 100.

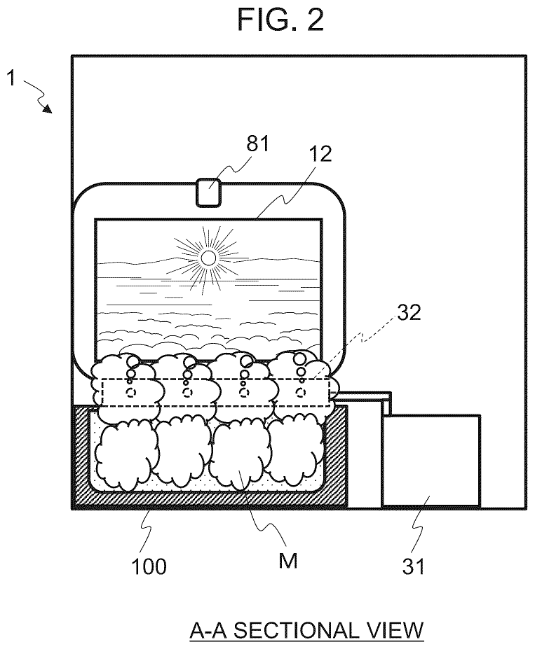

FIG. 2 is an A-A sectional view of the system 1 illustrated in FIG. 1B.

As illustrated in FIG. 2, a video of a scene particularly such as a sunset cloud sea is desirably used as the visual content displayed on the liquid crystal display. The cloud sea displayed on a lower side of a screen and the mist discharged from the mist generator 32 are integrated to produce a compound visual effect, so that the user is provided with an experience of enhanced realism.

In addition, the smart speaker 61 generating the sound stimulus with the gamma frequency using a function of speakers 63 enables treatment with a combination of the light stimulus and the sound stimulus.

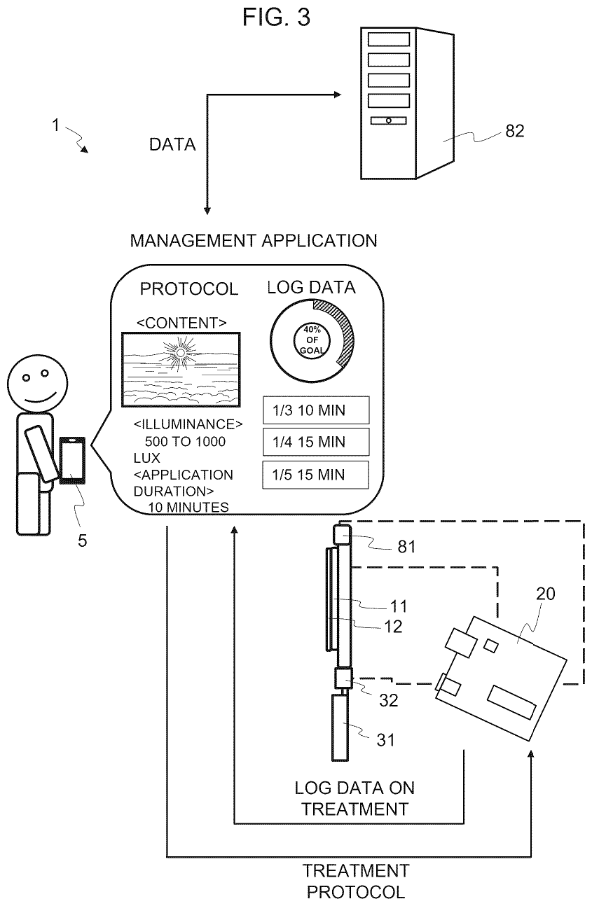

FIG. 3 is a diagram illustrating a flow of data in starting use of the system 1 illustrated in FIG. 1A and FIG. 1B.

As illustrated in FIG. 3, as an advance preparation to use the system 1, the user launches a management application on a user terminal 5 and sets a protocol for the treatment with the light stimulus and the sound stimulus (a treatment protocol). The treatment protocol includes visual content to be displayed on the liquid crystal display, an amount of the mist to be generated, sound content to be used as the sound stimulus, a duration of the treatment, and the like. Data on the treatment protocol is sent to a controller 20 in advance.

After entering the bathtub 100, the user gives the smart speaker 61 of a bath pillow type instructions to start the therapy and operates devices around the user (a lighting instrument, a treatment light source, the liquid crystal display, a valve) through the communicating function of the smart speaker 61, thus receiving the treatment with the light stimulus.

The system 1 first turns off illumination of the bathroom and then uses the optical sensor 81 to check that there is no optical noise that influences the therapy.

Next, the controller 20 of the treatment light source and the liquid crystal display applies the light stimulus with the gamma frequency from the light source 11 and displays the visual content on the liquid crystal display. In addition, the controller 20 gives instructions to open an IoT valve to start supply of hot water, thus discharging mist from the mist generator 32 toward the bathtub 100.

In addition, the speakers 63 provided in the smart speaker 61 generating the sound stimulus with the gamma frequency enables treatment with the combination of the light stimulus and the sound stimulus. Noise that spoils an effect of the sound stimulus can be detected by microphones 64 of the smart speaker 61 and reduced by a noise canceling function of the speakers 63.

A log of the treatment including time points of performing the treatment with the light/sound stimuli and the like is sent from the controller 20 to the management application and saved in a cloud server 82 through an Internet link.

Since the system 1 outputs the light stimulus from the liquid crystal panel, it is possible to display various visual contents that are tailored to differences in users' preferences or changes in the user's preference. It is known that, in general, a person continuously given the same visual stimulus becomes visually accustomed to the visual stimulus, thus declining an attention to (getting tired of) an object. In contrast, the system 1 can provide visual contents that are tailored to various changes in preference by operating the liquid crystal panel to dynamically change a transmission pattern of light.

Although the system 1 can display various visual contents on the liquid crystal panel, assuming that a size of the light source 11 is 1 m×0.6 m (the same size as a 46-inch television), a luminance of about 1200 cd/m² or more is required with consideration given to a prevention effect of the light stimulus on dementia.

With consideration given to the fact that a maximum luminance of a typical work display is about 300 cd/m², the user will watch a displayed visual content that will significantly dazzle the user.

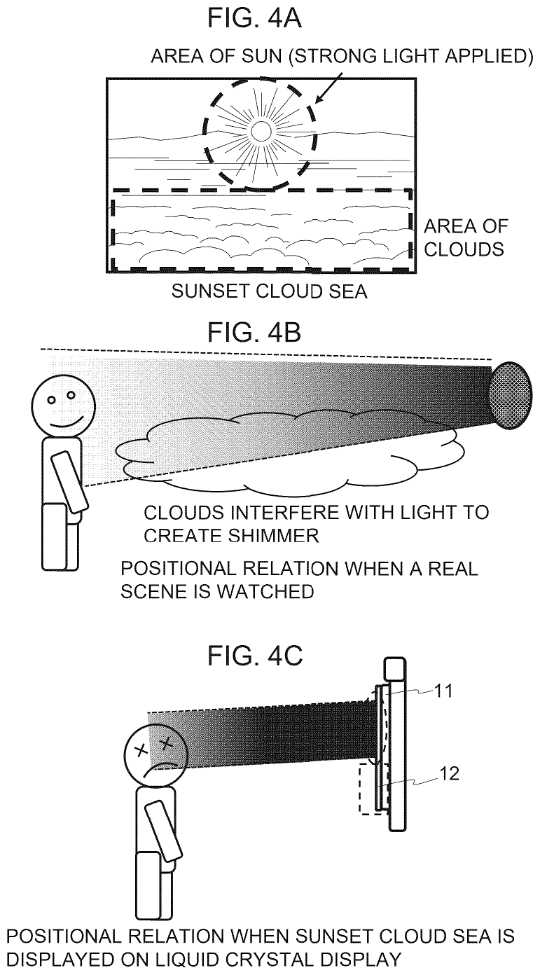

FIG. 4A is a diagram illustrating a sunset cloud sea. FIG. 4B is a diagram illustrating positional relation where a real sunset cloud sea is watched. FIG. 4C is a diagram illustrating positional relation where the sunset cloud sea is displayed on the display.

As illustrated in FIG. 4A, as a scene that a person finds agreeable even if strong light is applied, for example, a sunset cloud sea is known. As illustrated in FIG. 4B, although strong light is applied from an area where the sun is seen, the light undergoes diffuse reflection to shimmer in an area of clouds forward of the area where the sun is seen, and thus the scene can be watched as an agreeable scene.

In contrast, as illustrated in FIG. 4C, in a case where a similar visual content is displayed on the liquid crystal display, the strong light from an area where the sun is displayed is directly applied to the user because clouds are present on the same plane as a display panel.

### (1-2) Overview of Effects of System 1

Hence, the system 1 includes, in addition to the liquid crystal display, a fine particle discharge device 30 that discharges fine particles such as mist. Therefore, it is possible to cause light applied from the display to shimmer in a three-dimensional manner, thus providing the user with the same favorable visual effect as a shimmering natural scene. That is, the user can prevent dementia while enjoying without feeling discomfort.

As seen from the above, the present invention solves the problems of getting tired of a light stimulus and discomfort with the light stimulus by using various visual contents provided by a visual output device and the light shimmering in a three-dimensional manner provided by the mist (the fine particles) and enables the user to continue the treatment with the light stimulus without stress, thus preventing dementia.

The system 1 can be used for a therapy for, in addition to Alzheimer-type dementia, other types of cranial nerve disease and mental disorders such as Parkinson's disease and depression. For example, it is demonstrated that a stimulus with the gamma frequency to skin is effective in improvement in motor symptoms of Parkinson's disease. The system 1 can give the user a cutaneous stimulus with a cutaneous stimulus device 70 described later. Thus, using the system 1 may further enhance therapeutic effects.

### (2) Configuration of System 1

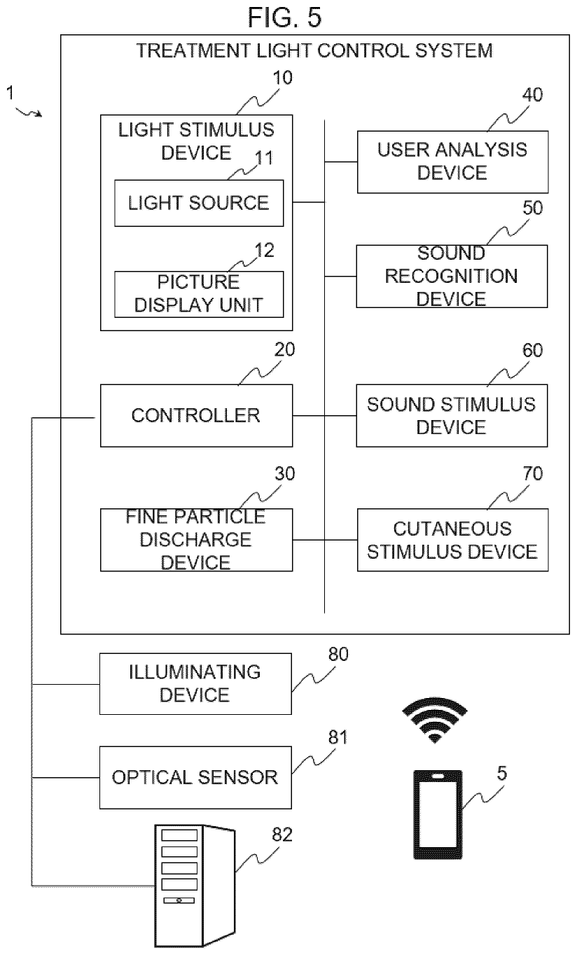

A configuration of the system 1 will be described. FIG. 5 is a block diagram illustrating the configuration of the system 1 illustrated in FIG. 1A and FIG. 1B.

As illustrated in FIG. 5, the system 1 includes a light stimulus device 10, the controller 20, the fine particle discharge device 30, a user analysis device 40, a voice recognition device 50, a sound stimulus device 60, and the cutaneous stimulus device 70. The system 1 has wired or wireless communication connectivity with the illuminating device 80, the optical sensor 81, and the cloud server 82. The system 1 also has wireless communication connectivity with the user terminal 5 used by the user.

### (2-1) Configuration of Light Stimulus Device 10

The light stimulus device 10 is a visual output device that includes the light source 11 and the visual display unit 12.

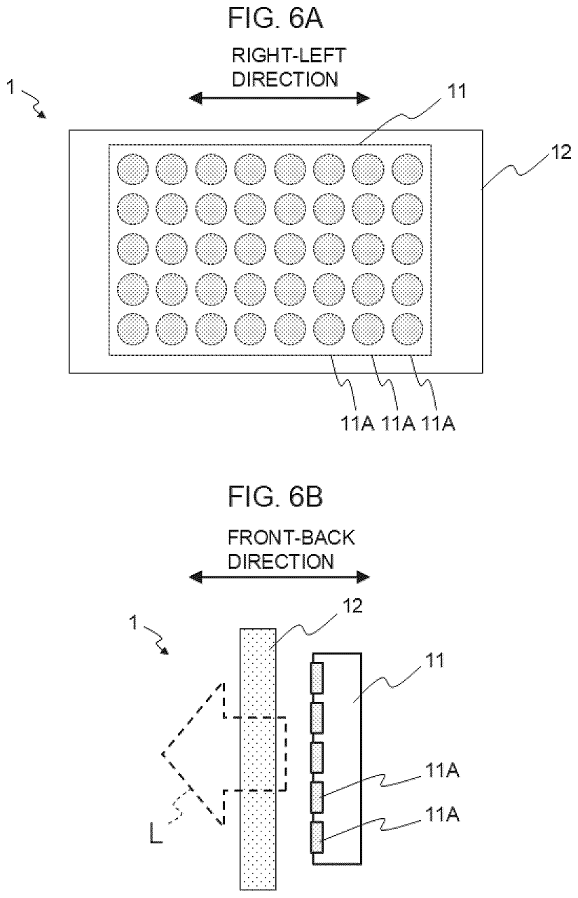

The light source 11 of the light stimulus device 10 applies blinking light mainly forward. As the light source 11, a panel-shape LED light source can be used. FIG. 6A is a diagram illustrating the panel-shape LED light source of the light stimulus device 10. FIG. 6B is a diagram illustrating the panel-shape LED light source of the light stimulus device 10.

As illustrated in FIG. 6A and FIG. 6B, the panel-shape LED light source has a structure in which a large number of LEDs are disposed vertically and horizontally in a grid pattern. The panel-shape LED light source applies light mainly forward (in a direction in which the user is positioned). That is, the light source 11 occupies a certain region orthogonally mainly to a direction of the application.

As the light source 11, an LED array in which LEDs are disposed in a grid pattern over its entire panel is desirably adopted so that the light source 11 is capable of applying a strong light stimulus. Using the LED array as the light source 11 results in less loss of light compared with, for example, a structure in which light is applied via a scattering plate, a reflecting plate, and the like, which will be described later, and enables high-luminance LEDs to be disposed with high density.

As illustrated in FIG. 1A and FIG. 1B, the visual display unit 12 of the light stimulus device 10 is disposed forward of the light source 11. The visual display unit 12 transmits light applied from the light source 11 to display a predetermined visual content on a surface. The visual display unit 12 displays various visual contents that are tailored to various preferences of the user, and the mist causes the light to shimmer in a three-dimensional manner. By these means, the user can comfortably accept the light stimulus while enjoying realistic changes.

The liquid crystal display used as the visual display unit 12 controls a pattern in which the light is transmitted. Liquid crystal displays are roughly classified by structure into two types: passive-matrix and active-matrix, and an active-matrix liquid crystal display, which is excellent in responsiveness, is desirably used.

In an active-matrix liquid crystal panel, each pixel is provided with an active element (a transistor). Each transistor is connected to an X electrode that performs switching of the transistor and a Y electrode that turns on pixels on a column basis, and a display level of each pixel can be adjusted with a voltage applied to a corresponding X electrode. In addition, it is desirable to dispose a color filter on a display side to enable color display.

### (2-2) Configuration of Controller 20

Next, a configuration of the controller 20 will be described.

First, a hardware configuration of the controller 20 will be described.

### (2-2-1) Hardware Configuration of Controller 20

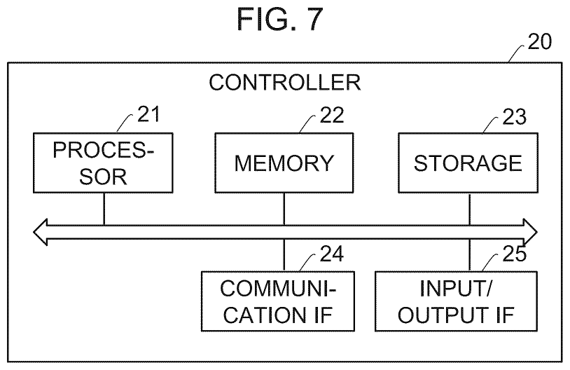

FIG. 7 is a block diagram illustrating the hardware configuration of the controller 20.

As illustrated in FIG. 7, the controller 20 includes a processor 21, a memory 22, a storage 23, a communication IF 24, and an input/output IF 25.

The processor 21 is a piece of hardware for executing a set of instructions written in a program stored in the memory 22. The processor 21 is constituted by an arithmetic unit, registers, peripheral circuits, and the like.

The processor executes a program pertaining to the management application to execute, for example, the following processes.
A process on information that is input into the user terminal 5 and relates to the light source 11, such as an amount of light and a wavelength
A process pertaining to generating a visual content to be displayed by the visual output device
A process pertaining to discharging fine particles from the fine particle discharge device 30
A process of determining controlled variables of each device based on user information input from the user analysis device 40

These processes will be described later in detail.

The memory 22 is for temporarily storing, for example, programs and data to be processed by the programs or the like. For example, the memory 22 is a volatile memory such as a dynamic random access memory (DRAM).

The programs include, for example, the following programs.
A program of operating system (OS)
A program that is downloaded and stored in the memory and executes control of each device
A program of a web browser that performs information processing

The storage 23 is a storage device for saving data. For example, the storage 23 is a flash memory, a hard disc drive (HDD), or a solid state drive (SSD).

The communication IF 24 is an interface through which the system 1 inputs and outputs signals to communicate with external equipment. As the communication IF 24, specifically, a module that supports a general-purpose telecommunications standard is desirably used.

The input/output IF 25 functions as an interface for an input device (e.g., a pointing device such as a mouse, a keyboard, etc.) for receiving input operations from the user and output devices (a display, the speakers 63, etc.) for presenting information to the user.

As the controller 20, for example, an open-source hardware, an industrial CPU board, or the like can be adopted.

### (2-2-2) Functional Configuration of Controller 20

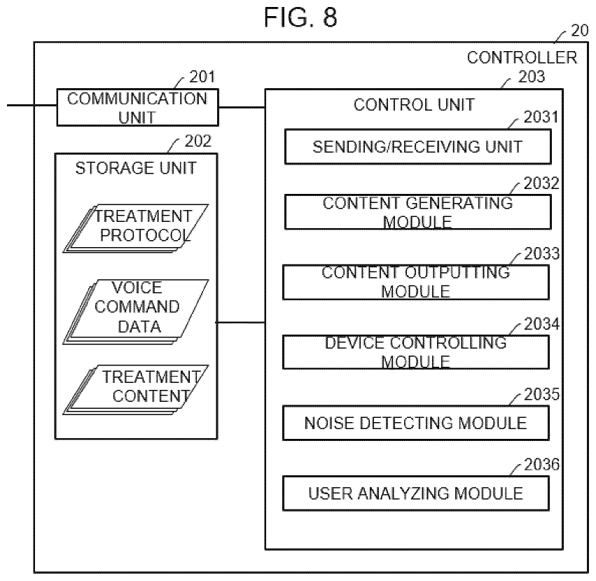

Next, a functional configuration of the controller 20 will be described. FIG. 8 is a diagram illustrating the functional configuration of the controller 20.

The controller 20 exercises functions as a communication unit 201, a storage unit 202, and a control unit 203.

The communication unit 201 performs a process for enabling the controller 20 to communicate with the external equipment.

The storage unit 202 stores data and programs to be used by the controller 20. The storage unit 202 stores a treatment protocol, voice command data, and treatment content.

The treatment protocol is data pertaining to details of control of each device in performing the treatment.

The voice command data is data in which an uttered voice of the user is associated with details of control of each device of the system 1.

The treatment content is data including visual content data (the light stimulus) and sound data (the sound stimulus) that are to be output to the user in performing the treatment.

The control unit 203 exercises functions as a sending/receiving unit 2031, a content generating module 2032, a content outputting module 2033, a device controlling module 2034, a noise detecting module 2035, a user analysis module 2036 as the processor 21 of the controller 20 performs processes under the programs.

The sending/receiving unit 2031 controls a process in which the controller 20 sends a signal to the external equipment under a communications protocol and a process in which the controller 20 receives a signal from the external equipment under the communications protocol.

The content generating module 2032 generates the treatment content to serve as the light stimulus and the sound stimulus. For example, in a case where the system 1 is used in a therapy for dementia, the treatment content is constituted by visual content data and sound data in which their intensities oscillate at a blinking frequency close to a frequency of a gamma wave. Note that the treatment content need not include the sound data. In this description, the blinking frequency denotes a frequency at which a highest peak is obtained in a frequency spectrum obtained by performing a Fourier transform on a waveform of changes in illuminance with time. The blinking frequency of the treatment content is preferably less than 100 Hz. That is, in the system 1, the light source 11 applies the blinking light with a blinking frequency of less than 100 Hz in at least a certain region of the light source 11.

That is, the content generating module 2032 converts frequencies of the visual content data and the sound data included in a video content to serve as materials into a blinking frequency of less than 100 Hz. The content generating module 2032 also converts the sound content into a sound signal that is modulated to a predetermined interval. The converted sound signal is output by the content outputting module 2033.

A method of generating the treatment content will be described later specifically.

Here, the system 1 may use the content generating module 2032 to generate the treatment content in advance. In this case, the content outputting module 2033 outputs, in a form of a visual content, a blinking visual signal from the visual output device.

On the other hand, in the system 1, a common video content may be output from the light stimulus device 10 and the sound stimulus device 60 by performing output control described later on the common video content with the content outputting module 2033. In this case, the treatment content need not be generated.

In a case where the treatment content has already been generated, the content outputting module 2033 outputs the treatment content as it is from the light stimulus device 10 and sound stimulus device 60.

In contrast, in a case where no treatment content has been generated, the content outputting module 2033 controls an output mode of the light stimulus device 10 and the sound stimulus device 60 when outputting a video content to serve as a material. That is, the content outputting module 2033 controls the output mode such that the blinking frequency of the light stimulus and the sound stimulus that are output from the light stimulus device 10 and the sound stimulus device 60 is less than 100 Hz, which is in a predetermined frequency band that is set in advance.

The content outputting module 2033 controls the blinking frequency of the light source 11 such that the blinking frequency is included in the predetermined frequency band that is set in advance.

In a case of giving light stimuli with different frequencies by adjusting intervals of turning on/blinking the light, ON/OFF control of the light source 11 is needed. In addition, means for adjusting an amount of the light while the light is turned on is needed.

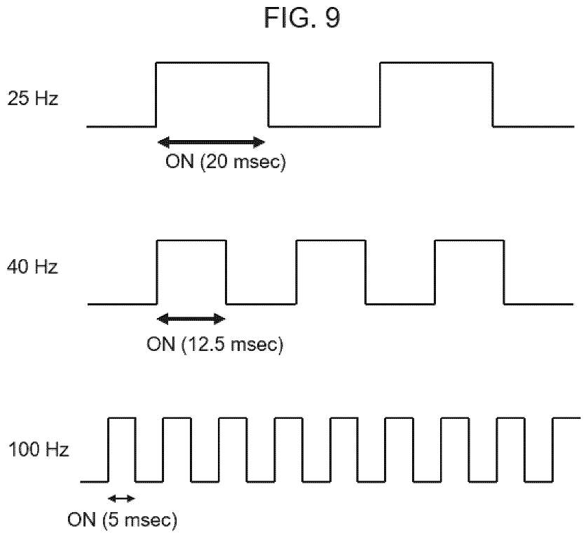

As a specific example, a control method in a case where the content outputting module 2033 gives the light stimulus in the gamma band of 25 to 100 Hz from the LED light source will be described. FIG. 9 is a diagram illustrating pulse waveforms of beams of light that are output at gamma frequencies. This diagram illustrates an example of waveforms of pulses with 25 Hz, 40 Hz, and 100 Hz when a Duty cycle (a ratio of an ON time within one period) is 50%.

As illustrated in FIG. 9, when turning on (ON) and turning off (OFF) alternate with a certain period, setting the (Duty cycle) to 50% causes the ON time to vary within the range of 5 to 20 msec. To adjust the amount of the light applied during this ON time, the following methods are conceivable.
A method of controlling current that flows through LEDs, organic EL (OLEDs), or the like
A method of adjusting a length of the ON time (the Duty cycle)
A method of performing PWM control during the ON time

When the current flowing through LEDs or organic EL (OLEDs), or the like is controlled stepwise, it is necessary to switch a circuit every time the current is changed, which complicates a control circuit.

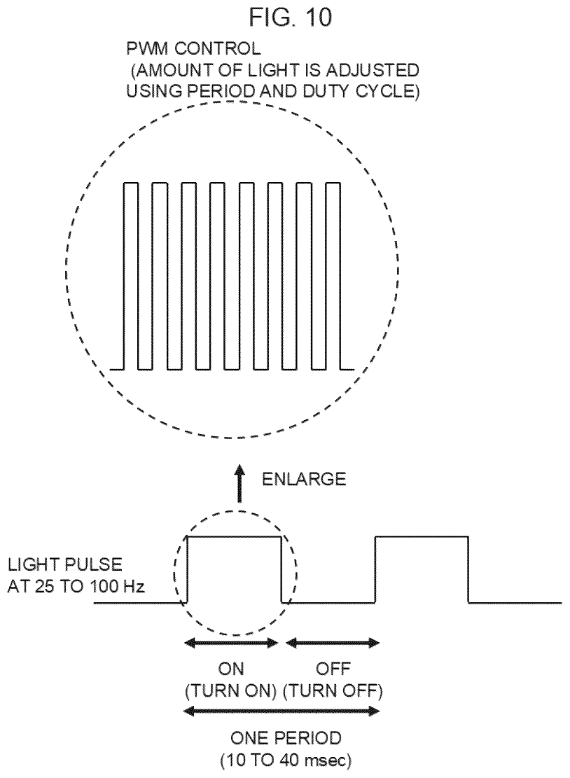

Therefore, the content outputting module 2033 performs the method of adjusting the length of the ON time and the method of performing the PWM control during the ON time. FIG. 10 is a diagram illustrating a pulse waveform of light that is generated by performing the PWM control while the light is turned on.

As illustrated in FIG. 10, in a case where the content outputting module 2033 generates the light stimulus with the gamma frequency while performing light amount adjustment by the PWM control, “light application by the PWM control” and “turning off” alternate every period of 10 to 40 msec. Typically, a frequency used in the PWM control is desirably 60 Hz or higher as with a display light source or the like.

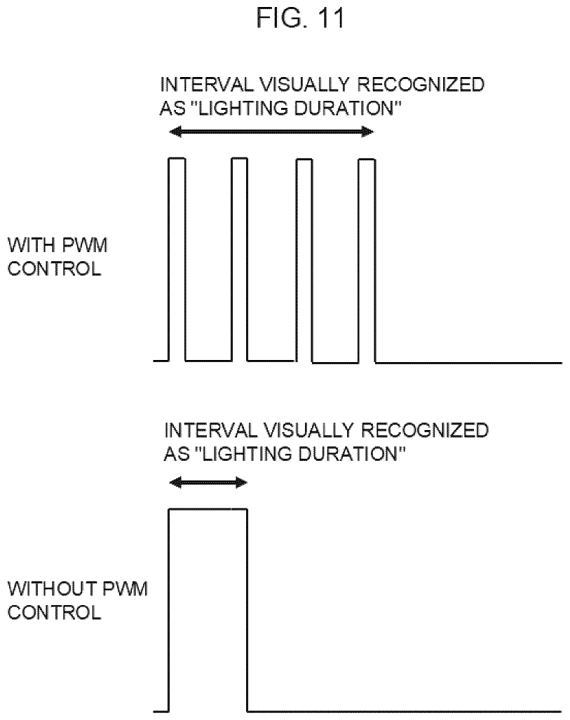

FIG. 11 is a diagram of a comparison of pulse waveforms between light amount adjustment by the PWM control and light amount adjustment by only an interval of the ON time.

As illustrated in FIG. 11, in a case where the light amount adjustment by the PWM control is not performed, an interval that is visually recognized as “lighting duration” is shortened compared with a case where the light amount adjustment by the PWM control is performed. This tendency increases when a small amount of light is needed, which shortens a time to recognize a visual content that is projected onto the display, and there is a possibility that the user cannot clearly recognize visual content.

Therefore, the light amount adjustment on the light source 11 by the content outputting module 2033 is desirably performed by the PWM control during the ON time. In addition, according to a past investigation about the light stimulus with the gamma frequency, a Duty cycle of light pulses is 50% or less. With consideration given to this, an interval of an OFF time is desirably at least not less than 5 msec, which is 50% of a period of 100 Hz.

That is, when the light stimulus with the gamma frequency is generated, it is desirable that a certain ON/OFF pattern of light on one period of 10 to 40 msec is repeated, and that the one period includes a “time during which turning off of the light continues for 5 consecutive msec or longer.” Such control by the content outputting module 2033 can be used for not only LEDs but also another type of the light source 11 such as OLEDs.

The device controlling module 2034 illustrated in FIG. 8 controls the devices constituting the system 1.

The control performed by the device controlling module 2034 includes the following.
Control of causing the fine particle discharge device 30 to discharge fine particles
Control of causing the cutaneous stimulus device 70 to output a cutaneous stimulus
Other control of driving various types of IoT devices used in the system 1

The noise detecting module 2035 uses sensing data from the optical sensor 81 to detect optical noise that penetrates into an environment (the bathroom) in which the system 1 is used.

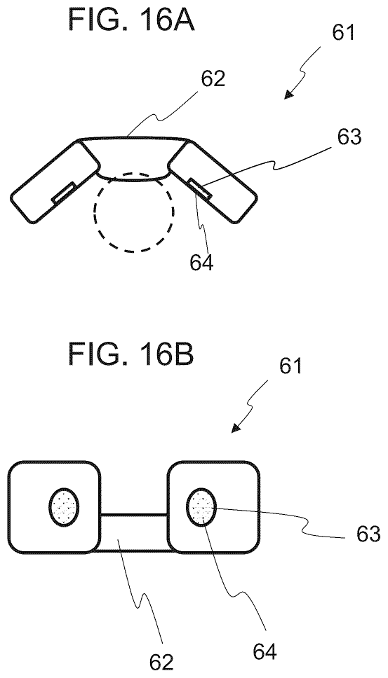

The noise detecting module 2035 also uses sensing data about sound sensed by the microphones 64 (see FIG. 16A and FIG. 16B) of the smart speaker 61 to detect sound noise that penetrates into the environment (the bathroom) in which the system 1 is used.

The user analysis module 2036 uses sensing data from the user analysis device 40 to analyze conditions and behavior of the user. A sensing process performed by the user analysis device 40 will be described later.

### (2-2-3) Method of Generating Treatment Content

Next, a method of generating the treatment content (the light stimulus, the sound stimulus) by the content generating module 2032 will be described.

First, as the visual content, a common still image such as a photograph, illustration, or painting, a visual content such as a movie or natural scene, or the like can be used as the content. An image file of a file format such as JPEG, PNG, GIF, PSD, or TIFF or a video file of a file format such as AVI, MOV, WMV, or MPEG can be used as the visual content. That is, the content generating module 2032 converts the visual content into a visual signal that blinks at a predetermined interval.

Together with the visual content, the sound content such as music, natural sound, radio, or PodCast can be used. A music file of a sound file format such as MP3, WAV, AIFF, AAC, FLAC, or Opus can be used as the sound content.

A method of generating the treatment content to be output with the gamma frequency by the content generating module 2032 will be described.

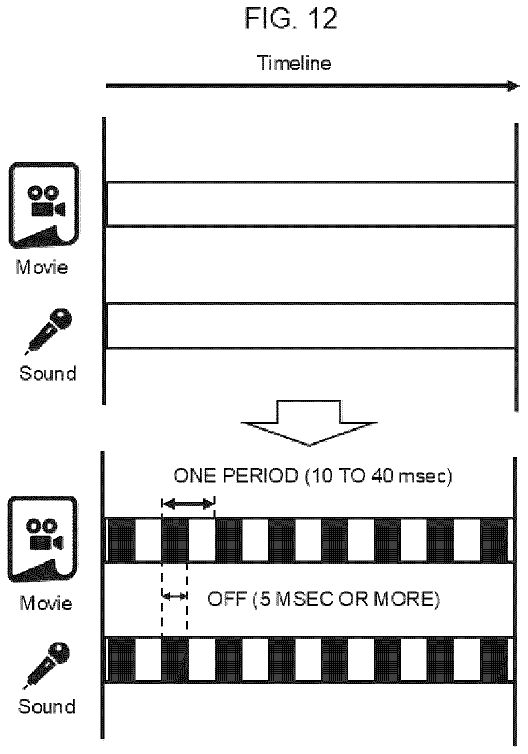

In a case of generating the light stimulus and the sound stimulus output with the gamma frequency, the content generating module 2032 performs the ON/OFF control on the light source 11 and a sound source and adjusts turning on/turning off of the light source 11 and an interval of ON/OFF of sound, thus generating light stimuli and sound stimuli with different frequencies. FIG. 12 is a diagram illustrating an example of the method of generating the treatment content.

The example illustrated in FIG. 12 illustrates an algorithm for generating the treatment content (the light stimulus, the sound stimulus) in the gamma band from video data with sound.

As illustrated in FIG. 12, in a case of, for example, generating content that gives the light stimulus and the sound stimulus in the gamma band of 25 to 100 Hz, a process of inserting intervals of light OFF (a black screen) and sound OFF at regular intervals of a period of 10 to 40 msec can be used. By these processes, common video content with sound can be converted into the treatment content (the light stimulus, the sound stimulus). In addition, by collectively performing the processes on the visual content and the sound in this manner, it is possible to make the light stimulus and the sound stimulus in phase easily.

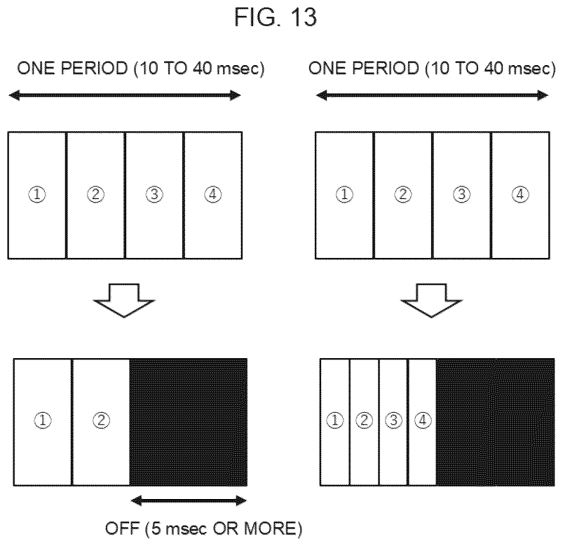

FIG. 13 is a diagram illustrating the process of inserting the light OFF and the sound OFF.

As illustrated in FIG. 13, there are two techniques shown below for the process of inserting the light OFF and the sound OFF.
A method of cutting an original visual content and sound and inserting OFF (see the left of FIG. 13)
A method of performing compression to increase a playback speed of the original visual content and sound to double speed and inserting OFF in accordance with a shortened time (see the right of FIG. 13)

Here, with consideration given to the recommendation that the Duty cycle of light pulses is 50% or less for the light stimulus with the gamma frequency, it is desirable that the interval of the OFF time is at least not less than 5 msec, which is 50% of the period of 100 Hz.

Next, a method of revising the treatment content will be described.

As a first example of revising the treatment content, timings of the light stimulus and the sound stimulus included in the treatment content can be shifted from each other when the treatment content is actually output from the system 1. In such a case, an inserting position of OFF of one of the light stimulus and the sound stimulus can be offset with respect to an inserting position of OFF of the other.

For example, in a case where there is a shift in phase of 5 msec between a nerve excitation of the user by the light stimulus and a nerve excitation of the user by the sound stimulus, induced nerve excitations can be made in phase by offsetting the sound stimulus with respect to the light stimulus by 5 msec. The shift in phase between the nerve excitations can be adjusted for each user because the lag varies among individuals.

Next, as a second example of revising the treatment content, a process of correcting a portion that is not suitable for the therapy will be described.

For example, there is a case where a luminance of part of a visual content of the treatment content is out of a range suitable for the treatment or a case where a sound volume of a part of the treatment content is out of a range that is set for the therapy. In such a case, using the treatment content may fail to provide intended effects.

Hence, the visual data and the sound data included in the treatment content are scanned, and an error portion in which the luminance or the sound volume is out of its appropriate range is revised such that the luminance or the sound volume falls under the appropriate range.

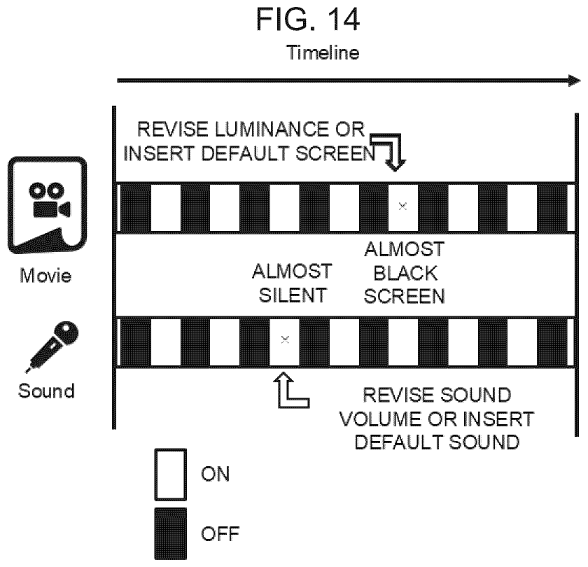

Specifically, as illustrated in FIG. 14, any one of processes shown below is performed. FIG. 14 is a diagram illustrating a process of detecting a portion that is not suitable for the therapy from the treatment content and correcting the portion.
Replace the error portion with a visual content or sound of original data (the video data with sound)
Revising luminance in the error portion
Revising sound volume in the error portion

Although the method of inserting OFF into both the visual and sound files is described as the method of generating the treatment content, the process of inserting may be performed on only one of the files.

Furthermore, regarding ON/OFF of the light and the sound, the method of newly generating data on the treatment content from the video data with sound is described. However, ON/OFF of the light and the sound may be performed by controlling the output devices (the visual output device and the speakers 63). For example, in a case where the visual output device includes a liquid crystal display with a backlight, it is possible to control ON/OFF of the backlight and the speakers 63 to output the treatment content, without processing the video data with sound.

When the backlight is turned on while the visual output device displays a black screen, optical noise occurs, which may reduce effects of the treatment.

Hence, it is desirable to use a system that controls turning on of a backlight to a visual content. A high-performance liquid crystal display performs such control that turns off its backlight in a region where a visual content is black. By using a similar system, it is possible to turn off a backlight at a timing to display a black screen, thus preventing optical noise from being produced.

### (2-3) Configuration of Fine Particle Discharge Device 30

The fine particle discharge device 30 illustrated in FIG. 5 discharges fine particles into a spatial region to which the light source 11 applies the light.

The fine particle discharge device 30 generates the fine particles by applying external energy to a liquid or a solid. The fine particle discharge device 30 applies energy to water, oil, and an inorganic substance to generate fine particles.

As the energy applied by the fine particle discharge device 30, various forms of energy such as ultrasound, electricity, and heat can be adopted.

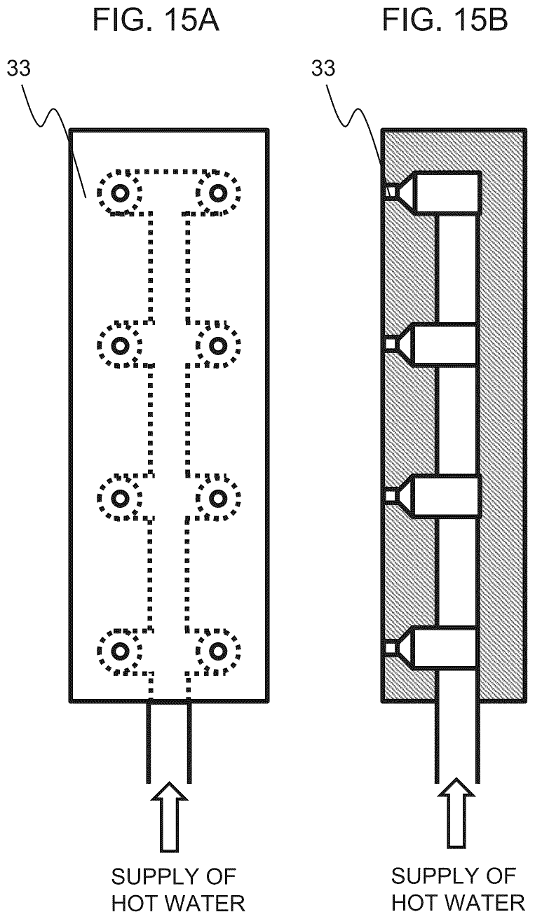

As illustrated in FIG. 1A and FIG. 1B, the fine particle discharge device 30 includes the mist generator 32 used in a steam sauna or the like, as an apparatus that generates mist. To the mist generator 32, a hot-water supplying unit 31 that retains hot water is connected. FIG. 15A is a diagram illustrating a structure of a head unit 33 of the mist generator 32 included in the fine particle discharge device 30. FIG. 15B is a diagram illustrating a structure of a head unit 33 of the mist generator 32 included in the fine particle discharge device 30.

As illustrated in FIG. 15A and FIG. 15B, the head unit 33 of the mist generator 32 is formed with a plurality of nozzles that atomize water and discharge the atomized water. For example, by being supplied with warm water, the head unit 33 discharges the mist from the nozzles. Note that the head unit 33 of the mist generator 32 may be supplied with cold water instead of the warm water.

Note that the hot-water supplying unit 31 of the mist generator 32 may be provided with a water supply valve as an IoT device having a communication controlling function pertaining to ON/OFF of warm water supply. With communication control of the warm water supply, it is possible to cause the mist generator 32 to automatically start generating the mist by using, for example, a voice recognition function of the user terminal 5.

Alternatively, the fine particle discharge device 30 may sprinkle warm water from a showerhead to generate fog in the bathroom, without discharging the mist from the mist generator machine. However, the mist is desirably used as the fine particles with consideration given to such a point that the mist provides a complex visual effect through Brownian motion.

### (2-4) Configuration of User Analysis Device 40

The user analysis device 40 illustrated in FIG. 5 has a function of detecting any one of a position, an attitude, a line of sight, and a hypnagogic state of the user.

The user analysis device 40 includes an image sensor such as a CMOS or a CCD. The user analysis device 40 includes, for example, a USB camera with approximately hundreds of thousands to ten million pixels.

The user analysis device 40 measures the position, movement of a body, an orientation of a face, open/close of eyes, and the like of the user and analyzes conditions of the user.

By analyzing the conditions of the user, the user analysis device 40 can check whether the user averts the user's eyes from the light stimulus or not and whether the user closes the eyes or not, thus evaluating whether the treatment is performed appropriately.

In addition, as the user analysis device 40, a radio wave sensor that detects a blink of the user can be used. In this case, there is no need to obtain an image like a camera, thus providing an advantage from a viewpoint of the user's privacy and the like.

Alternatively, as the user analysis device 40, a system that grasps a position of the user with an infrared distance sensor, a photo interrupter sensor, an ultrasonic motion sensor, or the like, a system that measures an aortic pulse wave to predict falling asleep of the user, or the like can be adopted.

Sensors constituting the user analysis device 40 may be provided at positions on the visual output device and can be disposed at any positions in a room such as a lighting instrument or a window.

In a case where the system 1 is used in a bathroom as in the present embodiment, a sensor placed on the bathtub 100 may be used as the user analysis device 40, as means for monitoring conditions of the user under the treatment. For example, a sound sensor, a pressure sensor, and a temperature sensor that detects respiration, a pulse, body motion, a body temperature, and the like may be used as the user analysis device 40 to prevent an accident such as dozing during bathing. This makes it possible to perform a process of emitting an alarm sound or calling for a safety check from the speaker 63 when an anomaly is detected in the user's heart rate, breath sound, body temperature, or the like.

### (2-5) Configuration of Voice Recognition Device 50 and Sound Stimulus Means

The voice recognition device 50 has a function of recognizing an uttered voice of the user.

The sound stimulus means has a function of outputting a stimulus that has a certain periodicity and is input into the user via a sense of hearing.

In the present embodiment, the smart speaker 61 in which the voice recognition device 50 and the sound stimulus means are integrally formed will be described.

In the system 1, a smart speaker 61 of a bath pillow type having water proofing properties is desirably adopted as the smart speaker 61 that can be used in a bathroom. As illustrated in FIG. 1A and FIG. 1B, the smart speaker 61 is disposed in a part of the bathroom positioned at a head of the user in the bathtub 100. FIG. 16A is a diagram illustrating a structure of the smart speaker 61 of a bath pillow type. FIG. 16B is a diagram illustrating a structure of the smart speaker 61 of a bath pillow type.

As illustrated in FIG. 16A and FIG. 16B, the smart speaker 61 of a bath pillow type includes a support unit 62 that supports the head and a pair of sound outputting units that are disposed on both sides of the head.

The pair of sound outputting units each includes the speaker 63 and the microphone 64 built in. A pair of left and right speakers 63 are disposed at positions close to ears of the user using the system 1. The sound stimulus means includes a sound sensor built in.

As seen from the above, since the smart speaker 61 is disposed at a position at which the smart speaker 61 supports the head of the user using the system 1, it is possible to smoothly give the user the sound stimulus from the positions close to the ears of the user.

The smart speaker 61 desirably has a shape such that the ears of the user are covered with the smart speaker 61 from the right and left as illustrated because the sound stimulus should be given to the user under conditions that sound noise is excluded as much as possible.

The smart speaker 61 causes a processor built in to execute voice application software to exercise at least functions shown below.
A sound operating function of recognizing an operational instruction given by the user's utterance
A function of remotely controlling the devices and peripheral IoT equipment

These functions enable the user to give instructions to start the treatment with the user's utterance, without using the user terminal 5.

In a case where the smart speaker 61 is provided with a biometrics function such as a voiceprint authentication function, the smart speaker 61 can identify the user who intends to use the system 1, and thus it is possible to retrieve a treatment protocol customized to the user.

Note that instead of the smart speaker 61 of a bath pillow type, voice recognition means and the sound stimulus means may be provided as individual devices. For example, in addition to the voice recognition means provided with the microphone 64, headphones or earphones having water proofing properties, a bone conduction speaker 63 having water proofing properties, or the like may be used as the sound stimulus means.

The smart speaker 61 may be provided with, for example, an ultrasonic motion sensor, an infrared sensor, a motion detection sensor, a contact sensor, and a strain sensor that detects a change in shape of a device caused by contact with the user. By using these sensors to detect that the head of the user is in the vicinity of the speakers 63 and confirm that a position of the head of the user is appropriate, it is possible to prevent such a trouble that the sound stimulus is excessive because the ears of the user are too close to the speakers 63.

### (2-6) Configuration of Cutaneous Stimulus Device 70

The cutaneous stimulus device 70 outputs an electric or ultrasonic stimulus that has a certain periodicity and is input into the user via a sense of touch. Note that the cutaneous stimulus device 70 may combine the electric stimulus and the ultrasonic stimulus.

As the cutaneous stimulus device 70, a device that provides a therapy for a neurological disorder using an electric stimulus can be adopted. Such a device includes devices of various structures such as a watch type, an earphone type, a headband type, a headgear type, a glasses type, a nose plug type, a mouthpiece type, and a patch type.

In particular, in a case where a device that gives an electric stimulus with the gamma frequency is adopted as the cutaneous stimulus device 70 and combined with the therapy by the light stimulus and the sound stimulus with the gamma frequency, it is possible to induce oscillation (neural oscillation) that produces an effect in prevention of and a therapy for dementia and improvement in cognitive ability. In fact, there has already been an actual treatment in which an electric stimulus with 40 Hz is given to a head of a subject using these devices, aiming at an improvement effect in cognitive ability.

### (3) Other Devices and Software

Other devices and software that are used together with the system 1 will be described.

### (3-1) Configuration of Illuminating Device 80

The illuminating device 80 in a space (the bathroom in the present embodiment) where the system 1 is provided is an IoT device that is remotely operated through a communicating function. The illuminating device 80 is operated by an input into the user terminal 5 through a communicating function that supports various telecommunications standards such as Bluetooth®.

When the light stimulus with the gamma frequency is given using the system 1, it is necessary to turn off a room illumination to avoid optical noise. Therefore, to save time and trouble of turning off the room illumination manually by the user, the illuminating device 80 as an IoT device with a communicating function is used.

### (3-2) Configuration of Optical Sensor 81

A space where the system 1 is provided is provided with the optical sensor 81. The optical sensor 81 has a communicating function and measures, in the space where the system 1 is provided, an amount of light in a space where the user receives the treatment when the illumination is turned off (i.e., the optical noise). The optical sensor 81 is an IoT device that sends detected data to the user terminal 5 through the communicating function.

Note that the optical sensor 81 may be built in any one of devices that are used together with the optical sensor 81, such as the liquid crystal display, the sound stimulus means, and the lighting instrument.

### (3-3) Configuration of User Terminal 5

The system 1 may be operated with the user terminal 5. The user terminal 5 is a terminal that can be operated with various pieces of application software installed on the user terminal 5. Specifically, as the user terminal 5, a desktop PC, a smartphone, a VR device, an AR device, or the like can be adopted. Examples of the pieces of application software installed on the user terminal 5 include the following.
A management application that manages the system 1
A voice application that manages the smart speaker 61 (the voice recognition device 50, the sound stimulus device 60)
An operating application that remotely operates other IoT devices

As the user terminal 5, a terminal that is provided with a graphical user interface (GUI) such as a touch panel for operating the applications, checking data, and the like is desirable. With consideration given to its easiness in operation and penetration rate, a general-purpose smartphone is suitable.

### (3-4) Waterproof Means

The system 1 may be also provided with waterproof means. Specifically, the light source 11 and the visual display unit 12 desirably have waterproof and dustproof functions. Therefore, it is desirable to use, for example, a hermetically sealed structure that is used for an outdoor display.

### (3-5) Antifog Means

The system 1 may be also provided with antifog means. The visual content can be clearly displayed when no water droplets are formed on the display surface, and thus it is desirable to take, for the visual display unit 12, countermeasures against fogging caused by adhesion of the mist generated by the mist generator 32. Therefore, it is desirable to use a film or coating agent having hydrophilicity or hydrophobicity for the display surface of the visual display unit 12. For example, a film or coating having hydrophilicity that can make water droplets wet to flatten may be used.

### (3-6) Configuration of Management Application

Next, the management application of the system 1 will be described.

As an application that manages details of the treatment with the light stimulus and the sound stimulus, application software used in a PC, a smartphone, or the like can be used.

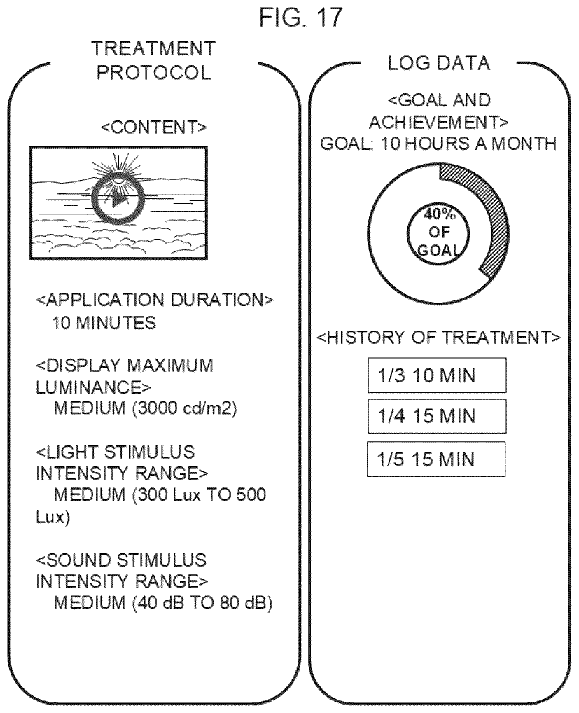

For example, application software that supports a general-purpose smartphone can be used as the management application. FIG. 17 is a diagram illustrating an example of a display screen of the management application.

As illustrated in FIG. 17, the display screen of the management application desirably includes a menu for managing a protocol of the treatment such as content data used as the light stimulus and the sound stimulus and a menu for managing a log of the treatment.

The user selects the following details using a management menu of the treatment protocol.
Content data (a type of video data, image data) to be used in the treatment
An application duration
Ranges of luminance and intensity of the light stimulus output from the display
A range of intensity of the sound stimulus output from the speaker 63

With these details, the user can set the treatment protocol that conforms to a mode desired by the user.

The user can check a record of the treatment received by the user from the management menu of the log of the treatment. The user can also check, for example, a degree of achievement with respect to a target total time of the treatment.

This enables the user to enhance motivation to continue the treatment. The application desirably saves data in the cloud server 82 so as to make the management application available in a plurality of devices.

### (3-7) Cloud Server 82

The system 1 has communication connectivity with the cloud server 82 via a network.

The cloud server 82 stores at least a treatment protocol for each user and a log about the treatment.

### (4) Processing by System 1

Next, processing at a time when the user uses the system 1 will be described.

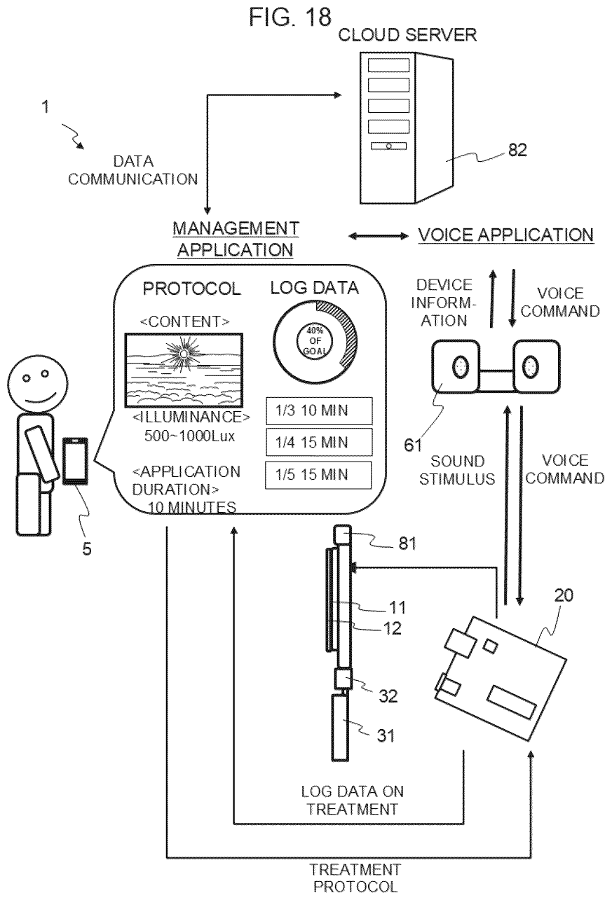

FIG. 18 is a diagram illustrating a communication state of data during the use of the system 1.

As illustrated in FIG. 18, the management application on the user terminal 5 sends, to the controller 20, a treatment protocol that is selected by the user and a voice command that is set, and the treatment protocol and the voice command are stored in a storage unit of the controller 20.

When a voice command is input into the smart speaker 61 with an uttered voice from the user, the voice application inputs the voice command to the controller 20. The controller 20 controls the devices under the treatment protocol associated with the voice command.

The controller 20 outputs a log of the treatment to the management application.

The management application sends following items of data to the cloud server 82.
A log of the treatment
Data about the conditions of the user analyzed by the user analysis means

The cloud server 82 stores the sent items of data.

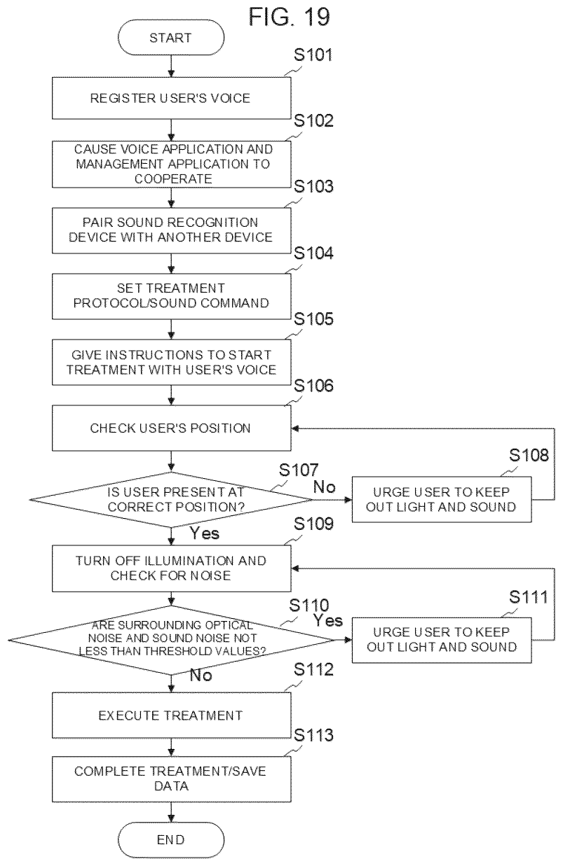

FIG. 19 is a chart illustrating a process in the use of the system 1.

First, the system 1 registers the user's voice (step S101).

Specifically, in order to enable the smart speaker 61 to identify the user, the user registers the user's voice in the voice application.

On the user terminal 5, the user logs in to the voice application on the smart speaker 61 (hereinafter, the voice application) and registers voice information on the user on the voice application running on the user terminal 5. For example, the user utters words specified by the voice application to the user terminal 5 to cause the voice application to store characteristics of the voice of the user.

The voice application performs data communication with the smart speaker 61 in wireless communication according to a general-purpose telecommunications standard. The voice application sends, to the smart speaker 61, the voice information on the user together with device information such as device names and addresses unique to the devices, and enables identification of the user by voice in subsequent operations.

After step S101, the user operates the user terminal 5 to perform cooperation between the voice application and the management application (step S102).

Specifically, the user causes the voice application to cooperate with the management application of the system 1 and associates the voice information on the user registered in the voice application to identification information on the user (a user ID unique to the user, etc.) in the management application.

By this operation, for example, when the user inputs instructions into the smart speaker 61 with an utterance, a treatment protocol corresponding to the voice can be retrieved. The voice application and the management application may be integrated into one application.

After step S102, the user operates the user terminal 5 to pair the smart speaker 61 with another device (step S103).

Specifically, the user pairs the smart speaker 61 with the controller 20, that is, makes communication settings to allow the smart speaker 61 to send a command so that the smart speaker 61 can send instructions to start the treatment to the controller 20 when the user gives the smart speaker 61 instructions with an utterance. In addition, the smart speaker 61 is also paired with other IoT devices (the water supply valve of the mist generator machine, the illuminating device 80, the optical sensor 81, etc.).

After step S103, the user operates the user terminal 5 to make settings of the treatment protocol and the voice command (step S104).

Specifically, the user logs in to the management application on the user terminal 5 and selects details of the treatment protocol (the visual and the sound content, the amount of the mist, the duration of the treatment, etc.).

For example, in a case where the user receives the treatment when bathing in the evening, selecting the visual content with a little blue, such as a “sunset glow” can prevent disruption of a circadian rhythm, which is a biological rhythm with a 24 hour period from being disturbed.

Next, on the management application, the user sets the voice command to retrieve the selected treatment protocol. A possible voice command is, for example, a combination of a name of the smart speaker 61 and the command, such as an utterance of the name of the smart speaker 61 followed by “start the treatment.” In addition, a command to suspend or finish the treatment may be set.

In the setting of the voice command, a plurality of combinations of treatment protocols and voice commands may be set to retrieve one of the treatment protocols corresponding to one of the voice commands.

The management application sends the set treatment protocol and voice command from the user terminal 5 to the controller 20. These items of data are saved in the storage unit of the controller 20, and the devices are prepared to start the treatment. Note that, in the second and subsequent uses of the system 1, processes of step S101 to step S104 can be omitted.

After step S104, instructions to start the treatment are given by the user's utterance (step S105).

Specifically, after entering the bathtub 100 in the bathroom where the system 1 is provided, the user inputs the voice command into the microphones 64 built in the smart speaker 61 with an utterance to give instructions to start the treatment. At this time, the voice application sends the voice command to the controller 20.

After step S105, the system 1 checks the position of the user (step S106).

Here, the following troubles are assumed when the position of the user is inappropriate.
An inappropriate position of the user may fail to give an effective light stimulus
An inappropriate position of the user may give an excessive sound stimulus

To prevent these troubles, the position of the user is sensed by a sensor provided in the smart speaker 61. The user analysis module 2036 of the controller 20 analyzes information from the sensing.

The sensor to check the position of the user may be provided in the light source 11, the room illumination, or the like or may be placed as an individual user analysis device 40. However, it is effective to provide the sensor in the smart speaker 61 that is close to the head of the user.

In step S106, in a case where the user is out of an appropriate position (No in step S107), the system 1 urges the user to move to the appropriate position (step S108).

Specifically, the user analysis module 2036 of the controller 20 causes the speakers 63 of the smart speaker 61 to output a voice alert indicating the position of the user is inappropriate. Afterward, the checking of the position of the user is performed again (step S106).

On the other hand, in a case where the user is at the appropriate position in step S106 (Yes in step S107), the system 1 turns off the illumination and checks for noise (step S109).

Specifically, the device controlling module 2034 of the controller 20 turns off the illuminating device 80. The noise detecting module 2035 of the controller 20 then obtains sensing data from the optical sensor 81 and checks that there is no optical noise that can interfere with the treatment.

The noise detecting module 2035 also obtains sensing data from the microphones 64 of the smart speaker 61 and checks that there is no sound noise that can interfere with the treatment.

If there is optical noise or sound noise that can interfere with the treatment (Yes in step S110), the noise detecting module 2035 outputs a voice alert to urge the user to keep out light and sound from the smart speaker 61 to urge the user to keep out light and sound (step S111). The sound noise may be reduced by the noise canceling function of the speakers 63.

On the other hand, in a case where there is neither the optical noise nor the sound noise that can interfere with the treatment (No in step S110), the treatment is executed (step S112).

Specifically, the content outputting module 2033 of the controller 20 outputs the light stimulus conforming to the protocol from the visual output device. Simultaneously, the content outputting module 2033 outputs the sound stimulus from the smart speaker 61. The content outputting module 2033 outputs the selected treatment content to the user according to the details set in the treatment protocol. In addition, the device controlling module 2034 of the controller 20 controls the fine particle discharge module to discharge the fine particles into the bathroom.

Here, while the treatment is executed, the user analysis module 2036 of the controller 20 analyzes the conditions of the user.

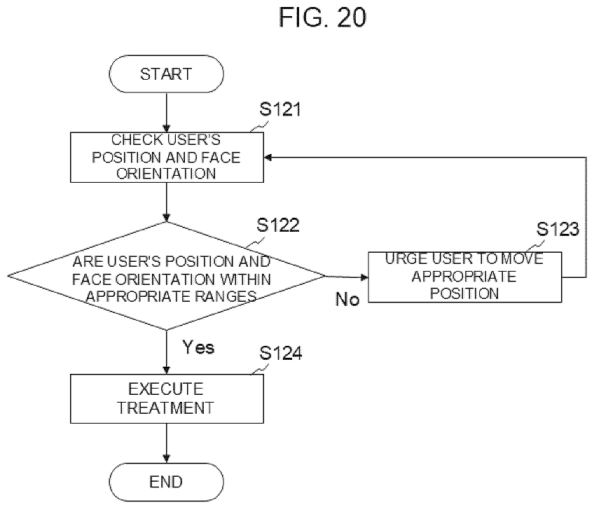

FIG. 20 is a diagram illustrating a process of analyzing the conditions of the user.

As illustrated in FIG. 20, the user analysis module 2036 checks the position of the user and the orientation of the face of the user (step S121).

In a case where the position of the user and the orientation of the face of the user are not within their respective appropriate ranges in step S121 (No in step S122), the user analysis module 2036 outputs an alert to urge the user to move to the appropriate position from the smart speaker 61.

On the other hand, in a case where the position of the user and the orientation of the face of the user are within their respective appropriate ranges in step S121 (Yes in step S122), the user analysis module 2036 does not output the alert.

Afterward, the treatment is continued by the content outputting module 2033 and the device controlling module 2034 of the controller 20 (step S124).

When the treatment corresponding to the treatment protocol selected in step S112 is finished, the system 1 completes the treatment and saves data (step S113).

Specifically, when the duration of the treatment that is set in the treatment protocol expires, the content outputting module 2033 of the controller 20 finishes outputting the treatment content. The control unit of the controller 20 generates a log of the treatment. The log is sent to the management application of the user terminal 5, and the sent data is saved in the cloud server 82 via the network.

With this above, the entire processing by the system 1 is finished.

Note that the system 1 described above using a bathroom may be used not only as dementia measures but also for treatment for other types of disorders in health conditions such as seasonal affective disorder (SAD), winter depression, jet lag, retinopathy, pain control, and wellness improvement.

### EXAMPLES

The present embodiment will be further described below based on examples. In the present example, an experiment for examining an effect of a size of the fine particles discharged from the fine particle discharge device 30 on alleviation of an adverse influence exerted by the light stimulus with the gamma frequency, such as discomfort (Experiment 1) was conducted. In the present example, an experiment for examining characteristics of an image that have an effect of alleviating the “discomfort” caused by the light stimulus with the gamma frequency (Experiment 2) was conducted.

### (Experiment 1)

Experiment 1 will be described below. As described above, Experiment 1 is the experiment for examining the effect of the size of the fine particles discharged from the fine particle discharge device 30 on alleviation of the adverse influence exerted by the light stimulus with the gamma frequency, such as discomfort.

### (Experiment Conditions)

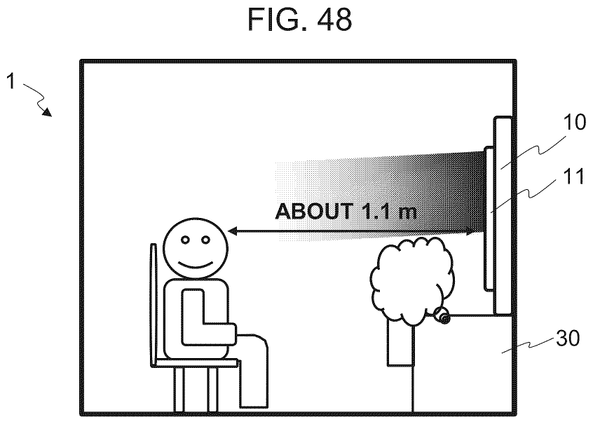

FIG. 48 is a schematic diagram of experiment conditions of Experiment 1. As illustrated in FIG. 48, in the present experiment, a fine particle generating device (the fine particle discharge device 30) that discharges four types of fine particles that are different in size was placed in a space that separates the treatment light source (the light source 11) and a subject. The treatment light source was placed at a position about 1.1 m away from positions of eyes of the subject. An amount of light of the treatment light source is set such that an illuminance measured at the positions of the eyes of the subject is about 400 Lux.

As the fine particles discharged between the treatment light source and the subject, fog produced from water was used.

In the experiment, the treatment was conducted under five conditions A to E about presence or absence of discharging of the fog and a particle size of the fog.
Condition A. Without discharging the fog, only applying the treatment light
Condition B. Applying the treatment light and discharging semi-coarse mist (average particle size 300 to 1000 μm) between the subject and the treatment light source
Condition C. Applying the treatment light and discharging semi-fine mist (average particle size 100 to 300 μm) between the subject and the treatment light source
Condition D. Applying the treatment light and discharging fine mist (average particle size 10 to 100 μm) between the subject and the treatment light source
Condition E. Applying the treatment light and discharging super fine mist (average particle size 10 μm or less) between the subject and the treatment light source

### (Experiment Equipment)

As the treatment light source, a COB panel light (12 V, 70 W, white light, manufactured by Zerodis), which is an on-board LED light source, was used. In the experiment, pulses with a Duty cycle of 50% are generated at 40 Hz as the light stimulus given to the subject, and an amount of the light was adjusted by the PWM control. In the experiment, the treatment light source was placed in a waterproof box and used to prevent the fog from adhering to a wire.

As the fine particle generating device, a device that atomizes and discharges supplied water was used. Note that atomizing devices used for the conditions B to E were different from one another. Specifically, to generate the semi-coarse mist in the condition B, a full cone spray nozzle, JJXP (manufactured by H. IKEUCHI & CO., LTD.) was used. To generate the semi-fine mist in the condition C, a full cone spray nozzle, J (manufactured by H. IKEUCHI & CO., LTD.) was used. To generate the fine mist in the condition D, a full cone spray nozzle, BIMJ (manufactured by H. IKEUCHI & CO., LTD.) was used. To generate the super fine mist in the condition E, an ultrasonic atomizer, IM4-36D/S (manufactured by SEIKO GIKEN INC.) was used.

To avoid an influence of the optical noise, light was kept out of a room where the experiment was conducted. Specifically, light was kept out such that an amount of light in the room was 1 Lux or less when the treatment light source was turned off.

### (Method of Measuring Effects)

Treatments under the conditions A to E were performed on three subjects for three cycles, five minutes for each cycle, in order of the conditions A, B, C, D, and E. Before the treatment under the condition A was performed, a five minute interval was provided in a state where the treatment light was turned off.

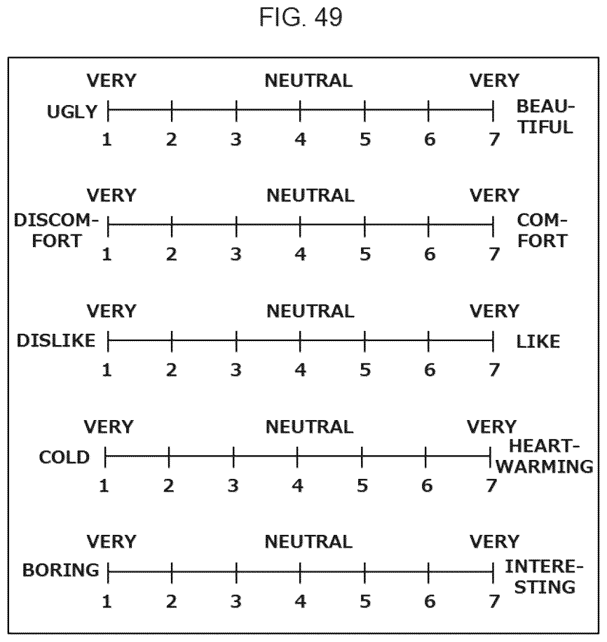

FIG. 49 is a diagram illustrating indices of subjective evaluation made by the subjects of Experiment 1. As illustrated in FIG. 49, as the indices of the evaluation, seven level evaluation was set for each of five items: “beauty,” “comfort,” “favorability,” “warmth,” and “interest.” In the experiment, the subjective evaluation of the conditions A to E was conducted in such a manner that the subjects answered a questionnaire illustrated in FIG. 49 for each of the conditions A to E after completion of the treatment for the first and third cycles.

### (Result of Experimental)

### (Differences in Appearance Among Different Average Particle Sizes)

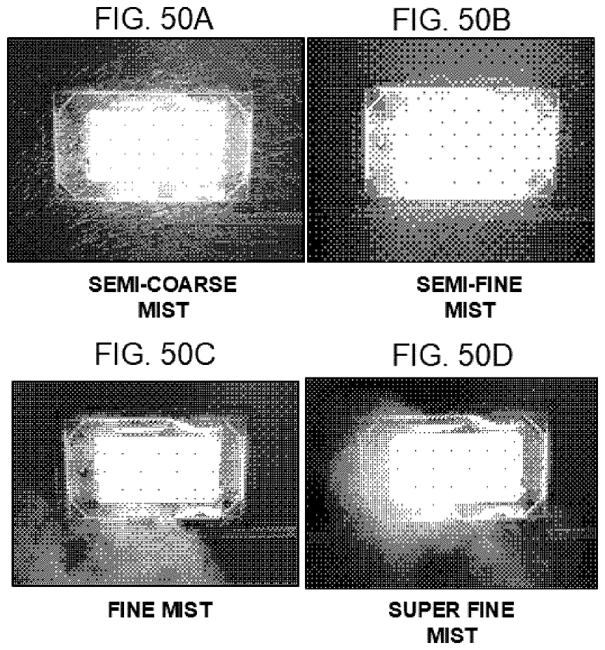

FIG. 50A is an image illustrating appearance of the fog under the condition B in Experiment 1. FIG. 50B is an image illustrating appearance of the fog under the condition C in Experiment 1. FIG. 50C is an image illustrating appearance of the fog under the condition D in Experiment 1. FIG. 50D is an image illustrating appearance of the fog under the condition E in Experiment 1. As illustrated in FIG. 50A to FIG. 50D, in cases of the semi-coarse mist and the semi-fine mist, the discharged mist falling down in water droplets like rain was observed. In contrast, in cases of the fine mist and the super fine mist, movement of the fog floating in the air was observed.

### (Results of Subjective Evaluation)

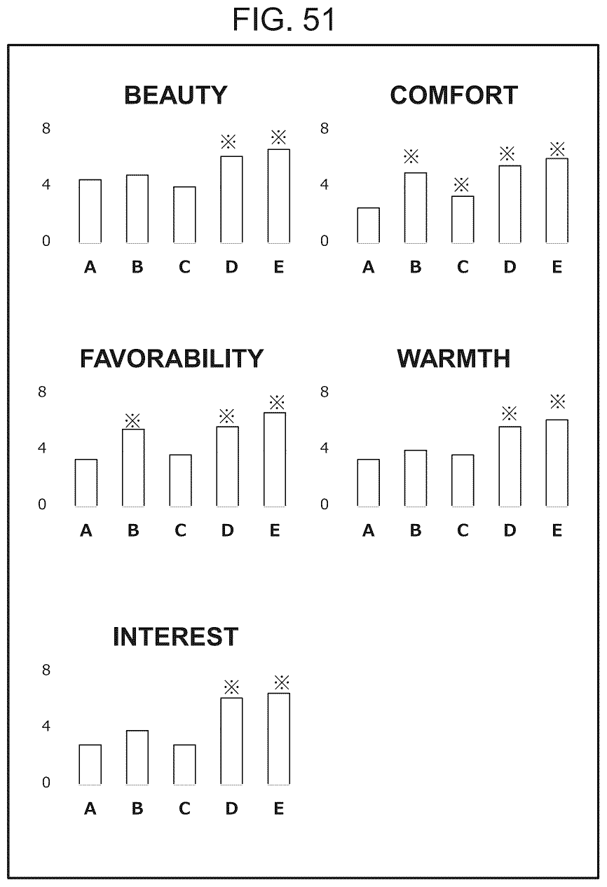

FIG. 51 is a graph illustrating average values of results of the subjective evaluation made by the subjects of Experiment 1 on a condition basis. In FIG. 51, conditions with “*” on bars indicating their values are conditions under which significant differences were recognized in evaluation results with respect to the condition A (only the treatment light) as a result of t test. As illustrated in FIG. 51, under the condition B (the semi-coarse mist), significantly higher ratings were obtained in two items “comfort” and “favorability,” while no significant differences were recognized in the other items. Under the condition C (the semi-fine mist), a significantly higher rating was obtained only in “comfort,” while no significant differences were recognized in the other items. Under the condition D (the fine mist) or condition E (the super fine mist), significantly higher ratings were obtained for all of the items.

The above results showed that using the fine mist or super fine mist having an average particle size of 100 μm or less provides a suitable aesthetic effect of scenery. A possible reason for this is that the fine mist or the super fine mist, which has the feature of “floating” in the air, can present an attractive appearance that resembles natural “shimmer”. In addition, it is known that fine particles having a size of several micrometers to tens of micrometers generally cause scattering of light called “Mie scattering” as with clouds floating in the sky. Therefore, an influence of the Mie scattering is also considered as a factor for why the fine mist or the super fine mist provided the suitable visual effect.

### (Experiment 2)

Subsequently, Experiment 2 will be described. As mentioned above, Experiment 2 is the experiment for examining characteristics of an image that have an effect of alleviating the “discomfort” caused by the light stimulus with the gamma frequency.

### (Experiment Conditions)

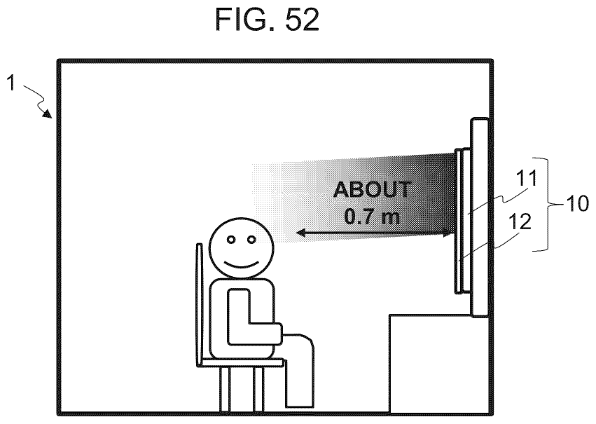

FIG. 52 is a schematic diagram of experiment conditions of Experiment 2. In the present experiment, four types of images are displayed on the display (the visual display unit 12), and effects that characteristics of the images displayed on the display exert on the subjects were compared. The treatment light source (the light source 11) was placed at a position about 0.7 m away from positions of eyes of the subject. An amount of light of the treatment light source is set such that an illuminance measured at the positions of the eyes of the subject is about 400 Lux. Note that an amount of light was adjusted for each image because a transmittance of light differs among the displayed images. The amount of light was adjusted by the PWM control.

### (Experiment Equipment)

In Experiment 2, a liquid crystal display extracted from a 43-inch television (manufactured by TOSHIBA CORPORATION) was used as the display. Behind the display, a COB strip light (24 V, white light, manufactured by BTF-LIGHTING), which is an on-board LED light source, was placed and used as the treatment light source. In the experiment, pulses with a Duty cycle of 50% are generated at 40 Hz as the light stimulus, and the amount of the light was adjusted by the PWM control.

In Experiment 2, four types of images: conditions A to D were used as the images for the experiment. As parameters that represent characteristics of each image, “luminance entropy” and “relative transmittance” were used. Definitions of the parameters and values of the parameters under conditions will be described below.

### (Luminance Entropy)

In a histogram representing, on a horizontal axis, luminances of pixels in an image, each of which is expressed in numeric values from 0 to 255, and on a vertical axis, a frequency of each luminance (its numerator is the number of pixels displayed at the luminance and its denominator is a total number of the pixels), a magnitude of variation in a distribution of the luminances is called luminance entropy. The luminance entropy H(P) is given by Equation 1. In Equation 1, P(A) denotes a probability of cocurrent of an event A.

[symbol]

In Equation 1, a base of the logarithm is two. Representing the probability, P(A) takes on a value of 1 or less. As a result, log P(A) takes on negative values. Thus, with a negative sign of the right side of Equation 1, the luminance entropy takes positive values. For example, an image that is entirely white, that is, an image all pixels of which have a luminance of 255 or an image that is entirely black, that is, an image all pixels of which have a luminance of 0 results in a luminance entropy of 0. A luminance entropy of an image increases with an increase in complexity of the image.

To calculate the luminance entropy, a program written in Python, a programming language, was used. In the program, a process of reading an image in a 256-level gray scale, a process of creating a histogram of gray level, a process of calculating frequencies, and a process of calculating the entropy were performed in this order. As a result of the calculation, values of the luminance entropy of the images under the conditions A to D (images A to D) were as follows.
Image A: 0
Image B: 4.3
Image C: 5.4
Image D: 7.7

### (Relative Transmittance)

In a case where an image is displayed on the display, and light is applied from the treatment light source placed behind the display, an image that results in a highest transmittance of light to the display is an image that is entirely white, that is, an image all pixels of which have a luminance of 255. When a transmittance of light in the case where the image that is entirely white is displayed on the display is assumed to be one, a transmittance of light in a case where a predetermined image is displayed on the display is defined as the “relative transmittance.”

The relative transmittance can be calculated by measuring an illuminance at a position at a certain distance from the display under a condition that the amount of light of the treatment light source is kept constant. In this experiment, the image A is the “image having a relative transmittance of one.” An illuminometer was placed at a position 2 m away from the display, and illuminances were measured. As a result, the illuminances of the images under the conditions were as follows.
Image A: 252 Lux
Image B: 230 Lux (relative transmittance 0.91)
Image C: 204 Lux (relative transmittance 0.81)
Image D: 50 Lux (relative transmittance 0.20)

### (Method of Measuring Effects)

In the experiment, a treatment in which an image was displayed on the display and the treatment light was applied to a subject for five minutes using the treatment light source disposed behind the display was performed. This treatment was performed for three cycles in order of images A, B, C, and D.

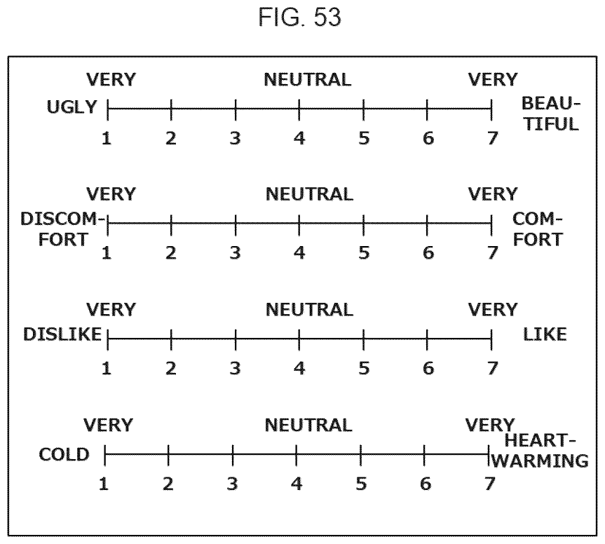

FIG. 53 is a diagram illustrating indices of subjective evaluation made by the subjects of Experiment 2. As illustrated in FIG. 53, as the indices of the evaluation, seven level evaluation was set for each of four items: “beauty,” “comfort, “favorability,” and “warmth.” In the experiment, the subjective evaluation of the conditions was conducted in such a manner that the subjects answered a questionnaire illustrated in FIG. 53 for each of the images A to D after completion of the treatment for the first and third cycles.

### (Result of Experimental)

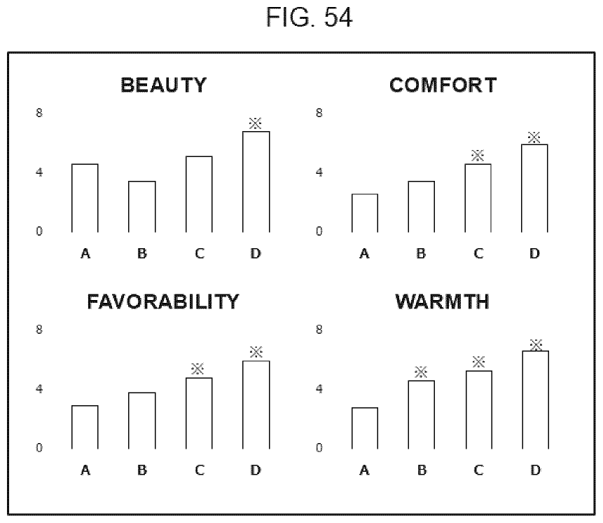

FIG. 54 is a graph illustrating average values of results of the subjective evaluation made by the subjects of Experiment 2 on a condition basis. In FIG. 54, conditions with “*” indicate that the respective images were images on which significant differences were recognized in evaluation results with respect to the image A as a result of t test. As illustrated in FIG. 54, the image B had a tendency to be lower than the image A in evaluation in “beauty.” In other indices, the image B had a tendency to be higher than the image A, but the image B obtained a significant difference only in “warmth.” With the image C, higher ratings than the image A were obtained in all the items, and significantly higher ratings were obtained in three items “comfort,” “favorability,” and “warmth.” With the image D, significantly higher ratings than the image A were obtained in all the items.

From the above results, the image B (entropy 4.3, relative transmittance 0.91) had a tendency to be lower than the white image in “beauty” and did not obtain significant differences in “comfort” and “favorability.” In contrast, the image C (relative transmittance 0.81, entropy 5.4) and the image D (relative transmittance 0.20, entropy 7.7) obtained higher ratings than the white image in all the items. As a possible cause of the low rating of the image B in “beauty,” it is considered that there is a possibility that the image B was visually recognized as an “unclear image with little variation in luminance.”

Therefore, from the above results, using an image having a luminance entropy of 5.4 or more and a relative transmittance of 0.81 or less compared with the white image is considered to be capable of giving a light stimulus favorable for the user.

### (5) Summary

As described above, in the system 1 according to the first embodiment, the light stimulus device 10 outputs the light stimulus to the user. Therefore, by raising concentration to reduce the number of blinks, it is possible to increase an amount of light entering eyes of the user per certain period of time, thus enhancing the effects of the treatment.

In addition, the system 1 generates the treatment content from a common video content, and thus it is possible to generate treatment content that each of users having different preferences finds agreeable.

In addition, in a case where the user gets tired of specific treatment content, it is possible to generate new treatment content in accordance with a preference of the user. That is, unlike conventional treatment light such as constant blinking light, it is possible to constantly give the user a light stimulus that is new and agreeable to the user, thus switching light stimuli from one to another. This makes it possible to provide treatment content that has high therapeutic effects and can be continuously used by the user.

In addition, the visual display unit 12 displays various visual contents that are tailored to various preferences of the user, and the mist produces a transmission/reflection pattern of the light changing in a three-dimensional manner and provides a target in addition to a surface of the display. By these means, the user can comfortably receive the treatment with the light stimulus while enjoying realistic changes.

In addition, the fine particles such as fog or bubbles discharged by the fine particle discharge device 30 undergo Brownian motion in a medium such as the air or water to scatter the light stimulus output by the visual output device, thus providing the user with visual “shimmer.” The Brownian motion is a phenomenon that is generally observed in the natural world but basically does not occur in an artifact. Thus, the fine particles that undergo Brownian motion to fluctuate have an effect of giving the user a suitable psychological influence such as “naturalness,” “relaxing effect,” and “recovery of attention.”

Therefore, the fine particles create a natural-like atmosphere. Thus, an effect of giving the user an impression of reality on the visual content can be expected even when a quality of the visual content (the light stimulus) output from the visual output device such as resolution is low. Although a device such as the liquid crystal display tends to deteriorate with strong light, the fine particles are generated and disappear on the spot, and thus the problem of the deterioration does not occur.

Unlike a flat display, the fine particles produce movement with depth. Thus, the user's perspective is less likely to remain fixed, and effect of recovery from eye fatigue or the like of the user looking at the light stimulus scattered by the fine particles is also expected.

In addition, a pattern made by light reflected by the fine particles constantly changes to provide visual effects caused by phenomena such as a “rainbow” or “eddy” at random timings, thus raising the user's expectation of aesthetic pleasure (activation of a brain's reward system) that occurs incidentally, and it is possible for the user to continue concentration.

Furthermore, by adding fragrance or medicine to the warm water supplied as the fine particles, it is possible to add smell or give a deodorization effect, sterilization effect, or the like, and thus air cleaning and deodorization effect can be also expected.

In addition, a stimulus that cannot be obtained only by the visual content, such as a tactile stimulus given by touching fine particles drifting toward the user on airflow in the space in use can be given to the user.

In a case where the system 1 is used in a bathroom, the user can prevent dementia as part of the user's usual practice without adopting a new practice because an action of getting into the bathtub 100 is included as part of the user's daily life. This point provides a great advantage from a viewpoint of saving the user's time or establishing a practice of the prevention because it is difficult for a busy user to create a new time for the prevention. In addition, using the system 1 in a bathroom provides an advantage also in that water or warm water being material of the fine particles can be easily obtained and there is no harmful influence of discharging the fine particles into the air.

In addition, since the system 1 includes the user analysis device 40, it is possible to check the position and the orientation of the face of the user before the execution of the treatment with the light stimulus or the like. When the position or the orientation of the face of the user is out of the range suitable for the treatment, it is possible to perform control of urging the user to move to the appropriate position or revising a position of the light source 11.

In addition, it is also possible to evaluate a degree of concentration of the user under the treatment by using the user analysis device 40 to measure a distance between the user and the light source 11 and adjust the amount of the light in accordance with the distance or to measure a frequency of the user's blinking.

### (6) Modifications

Modifications of the devices mentioned above will be described below.

### (6-1) Modifications of Light Source 11

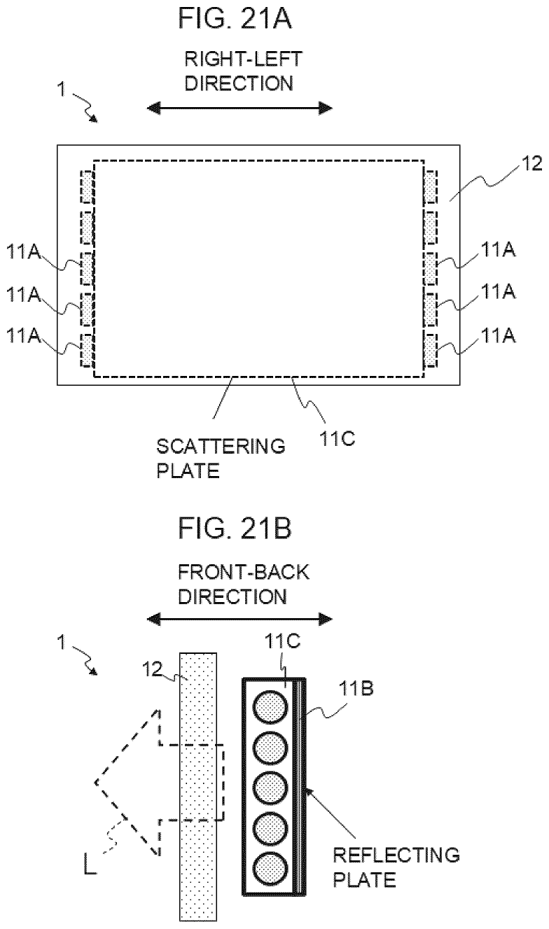

FIG. 21A is a diagram illustrating a modification of the light source 11. FIG. 21B is a diagram illustrating a modification of the light source 11.

As illustrated in FIG. 21A and FIG. 21B, the light source 11 according to the modification has a planar structure using a scattering plate 11C and a reflecting plate 11B.

In the light source 11, the scattering plate and the reflecting plate 11B are disposed side by side in a front-back direction.

On both lateral sides of the scattering plate, a plurality of LEDs are provided. By the scattering plate 11C scattering light from the LEDs and the reflecting plate 11B reflecting the scattered light toward the scattering plate 11C, the light source 11 applies the light forward as a whole.

Light having a plurality of wavelengths may be used for the light source 11. In this case, LEDs different in wavelength are desirably arranged evenly. In addition, LEDs different in wavelength may be disposed on four side surfaces of the scattering plate 11C: top, bottom, left, and right.

For example, in a case where blue, green, and red LEDs are used, the LEDs having the following peak wavelengths are desirably used.
Blue: at or around 480 nm (an effect can be expected in adjusting circadian rhythm, etc.)
Green: at or around 525 nm (an effect can be expected in pain control, etc.)
Red: at or around 670 nm (an effect can be expected in cell activation, etc.)

Organic light emitting diodes (OLED), in which an organic substance emits light, may be used for the light source 11.

Alternatively, laser diodes can be also used. For example, a method of forming a surface light source by vertically arranging structures each of which includes a combination of a laser diode and a light guide rod is available.

As the light source 11 other than that of LEDs or OLEDs, various types of the light source 11 such as a xenon lamp, a halogen lamp, a deuterium lamp, a mercury arc lamp, a excimer lamp, or an incandescence light bulb are available.

Instead of the light source 11 and the visual display unit 12, a light emitting display can be also used.

In a light emitting display, such as an organic EL display or a plasma display, pixels of the displays emit light. Thus, the display itself can be used as the light source 11.

In a plasma display, cells to which a phosphor is applied are disposed on a display surface in a grid pattern. In each cell, a discharge phenomenon is caused to occur to generate ultraviolet light, and the phosphor is excited by the ultraviolet light to emit light. By using a phosphor that emits light of a wavelength suitable for light treatment, the light can be effectively used as a light stimulus for the treatment.

In a case of using a field emission display, by using a phosphor that emits light of a wavelength suitable for light treatment as a luminophore that emits an electron beam when a voltage is applied to the luminophore, the light can be used as a light stimulus for the treatment.

Alternatively, a micro LED display, which has a structure in which an LED is disposed at each of its pixels, may be used.

Alternatively, a display that creates an afterimage to be visually recognized as a visual content by rotating an LED array at high speed and causing the LED array to emit light at timings at which the LED array passes a predetermined position may be used.

These types of displays can be used in a VR device or a three-dimensional display as well as being used as a display that performs 2D display, such as a television or a monitor for a PC. In this case, a three-dimensional visual content can be generated by displaying right-eye and left-eye visual contents.

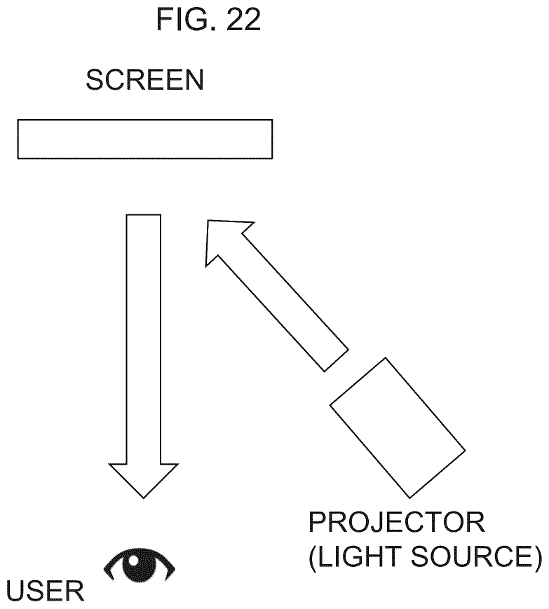

Alternatively, a projector that projects a visual content onto a surface of projection can be used as the light source 11. FIG. 22 is a diagram illustrating a positional relation in a case where a projector is used as the light source 11.

As illustrated in FIG. 22, in a case where a projector is used as the light source 11, the user visually recognizes a visual content projected onto a screen, a panel, or the like, rather than looking directly at the light source 11. As the projector, a CRT-type projector, which is an early projector that generates a visual content with its cathode-ray tube and projects the visual content onto a screen. In this case, the projector projects light of a wavelength suitable for the treatment onto the screen. To ensure an amount of light necessary for the treatment, a screen that causes strong reflection, such as a white screen, is desirably used.

Alternatively, the micro LED display may be used as the projector. In this case, a projector for which quantum photonic imaging (QPI) technology is employed is particularly preferable because the projector is small and capable of projecting a high-resolution, high-luminance visual content.

Each pixel of a QPI display has a structure in which three layers of light emitting element: red, green, and blue are stacked, includes a logic circuit on its bottom surface, and has a structure of guiding light on its upper surface. A suitable target of the projection is the user's hand, a translucent display of AR glasses, or the like.

In a case where the QPI display is used for the system 1, it is desirable to select and use light emitting elements of a wavelength suitable for the treatment.

Devices that reflect light at a position close to the user's eyes, such as AR glasses, are suitable when a small projector is used for the treatment because the devices enable the therapy to be performed with a smaller amount of light than a case where light is applied from a distance.

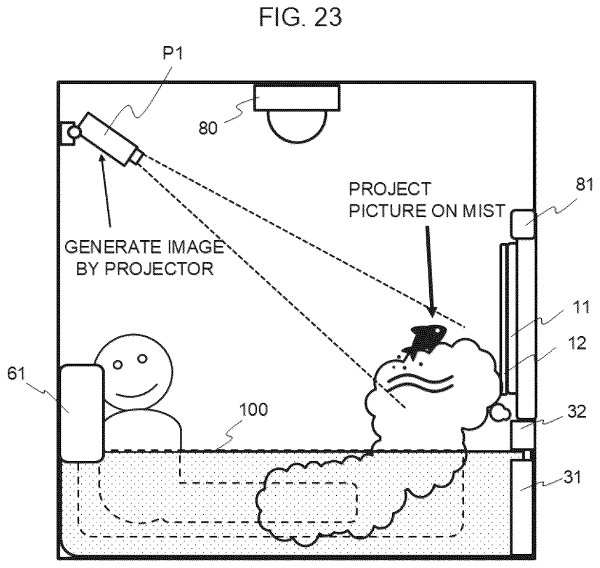

FIG. 23 is a diagram illustrating a state where the treatment light is projected from a projector onto the fine particles.

As illustrated in FIG. 23, together with output of the light stimulus from the visual output device, another visual content may be projected from the projector onto the fine particles. In this case, the visual content projected onto the fine particles serves as an additional light stimulus. When the treatment with the light stimulus with the gamma frequency is performed, it is desirable to blink the light stimulus projected by the projector and the treatment light source output from the visual output device in synchronization with each other.

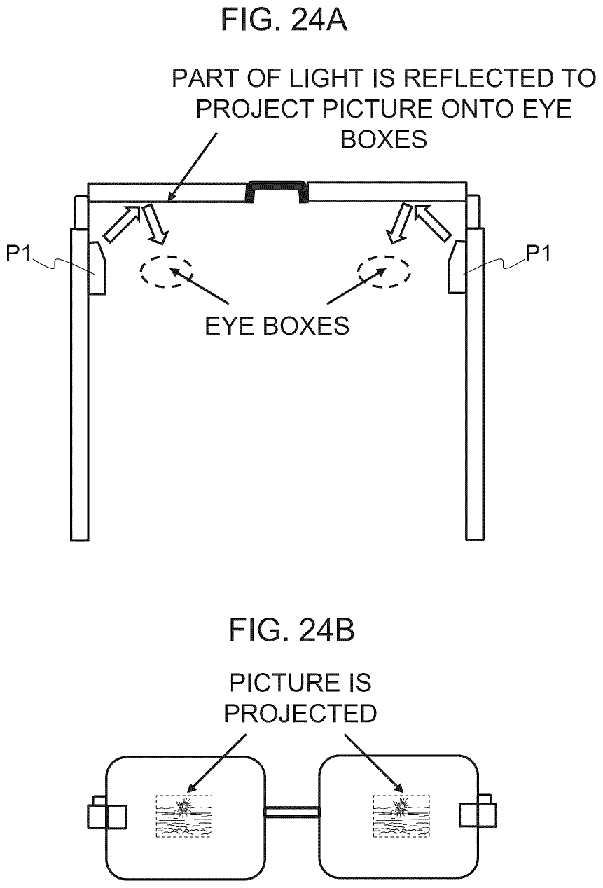

In a case where a small projector with micro LEDs or the like is used as the light source 11, a wearable device provided with small projectors, such as AR glasses, is included in the visual output device. FIG. 24 is a schematic diagram of AR glasses provided with small projectors.

As illustrated in FIG. 24, smart glasses that are provided with two small projectors on their side surfaces can be used as the visual output device.

The small projectors are disposed on lens sides of right and left temple parts of the glasses.

The small projectors apply beams of the treatment light toward lenses each formed of a partially transparent plate. The beams of the treatment light are reflected by the lenses and projected, in a form of a visual content, into eye boxes, which are spaces where the visual content can be visually recognized clearly.

Alternatively, a method in which, for example, a watch is provided with a small projector and a visual content is projected onto a palm or the like can be also used.

To the light source 11 described thus far, a near infrared light source of 700 to 1400 nm (typical peak wavelengths include 850 nm, 940 nm, and 1064 nm) may be added. Near infrared rays are known for exerting effects of health improvement, age control, improvement of a retinal condition, and the like caused by activation of mitochondrial function and the like.

### (6-2) Modifications of Visual Display Unit 12

As the visual display unit 12, a passive-matrix liquid crystal display may be adopted instead of the active-matrix liquid crystal display mentioned above. In the passive-matrix liquid crystal display, transparent electrodes are disposed sandwiching an alignment layer in which liquid crystal is enclosed. By adopting the passive matrix, a structure of the visual display unit 12 is simplified, which offers cost advantages. With a color filter being disposed on a display side, the passive-matrix liquid crystal display can be used as a color liquid crystal display.

Alternatively, an electro-wetting panel may be adopted as the liquid crystal display. In this case, transmission of light is controlled by applying a voltage to change a wettability of a surface of a water-repellent insulation layer in each pixel, thus changing a shape of oil included in the pixel.

When the voltage is not applied, the surface of the water-repellent insulation layer is covered with the oil, and light of a backlight is cut off. When the voltage is applied to transparent electrodes, the surface of the insulation layer turns hydrophilic to repel the oil, and thus the light of the backlight can be transmitted.

By switching ON/OFF of the transmission of the light for each pixel, a visual content can be displayed. In a case of performing color display, a method of using oils of a plurality of colors and a method of stacking color filters are conceivable.

Alternatively, an electrochromic panel may be adopted as the liquid crystal display. In this case, a wavelength of light to be transmitted is controlled by changing a color of each pixel with an electrical stimulus (application of a voltage).

The panel has a structure in which transparent electrodes vertically sandwich cells as with the passive-type liquid crystal display. By applying a voltage to the transparent electrodes, an oxidation reduction state of a color developing layer formed on the electrode changes to develop a color. By causing each cell to develop a color individually, it is possible to display an intended visual content.

As a material of the color developing layer, a metallic oxide is often used. As typical examples of the metallic oxide, tungsten oxide (WO₃), Prussian blue, NiO, Ir(OH)x, and the like are known.

There has recently been developed an electrochromic panel that includes different color developing layers being stacked and can express a wider range of colors by adjusting colors of the color developing layers. For example, a panel that includes a combination of a layer of stabilized vanadium oxide (SVO) and a layer of tungsten oxide and that changes a color tone two-dimensionally with a combination of voltages applied to the layers has already been successfully produced. Using such a technology, the wavelength of the light to be transmitted may be controlled finely.

The organic EL panel mentioned above itself serves as the light source 11. However, the organic EL panel has such a disadvantage that emitting strong light shortens a life of the organic EL panel. Therefore, the light source 11 may be provided separately, and an organic EL panel having transmittivity may be used as a liquid crystal display that transmits the treatment light applied from the light source 11.

In this case, as means for imparting the transmittivity to the organic electro-luminescence display, a method in which transparent electrodes vertically sandwich the organic EL display as with the passive-matrix liquid crystal display and a method of providing a transparent region that transmits light between every organic EL cell can be used.

To provide a gap between every organic EL cell, a conceivable structure is such that, for example, transparent cells are arranged so as to separate columns in which RGB cells are arranged.

As seen from the above, the liquid crystal display combined with the light source 11 in the visual output device can be used in a VR device or a three-dimensional display as well as being used as a display that performs 2D display, such as a television or a monitor for a PC. In this case, a three-dimensional visual content can be generated by displaying right-eye and left-eye visual contents.

Alternatively, the treatment light transmitted by a transmissive display may be displayed on a screen or a panel. In this case, the light source 11 and the transmissive display function as a projector.

In a case of using a projector including the transmissive display such as a transmissive liquid crystal panel, the light source 11 is disposed at a position of the light source 11 of the projector.

For example, in a case of a color projector including the transmissive liquid crystal panel, light applied from the light source 11 is split into red, green, and blue by a dichroic mirror, and visual contents of the colors made by the transmissive liquid crystal panel are combined and projected through a set of lenses.

For the color display, a method of turning on LEDs of different colors may be used, rather than splitting light with the dichroic mirror. Alternatively, a monochrome visual content may be projected. For the transmissive panel, visual display means other than the liquid crystal, such as the electro-wetting or electrochromic may be used.

As described above, the modification of the visual display unit 12 may be integrated with the light source 11 as with a common display and may be separated from the light source 11.

The light applied to a light transmission control unit may be light applied via a reflecting plate or the like, rather than light directly applied to the light source 11.

For example, it is conceivable to use, as the light stimulus device 10, a mechanism that controls a reflection pattern of light applied from the front, rather than transmitting light of a backlight, as with an electronic paper.

For example, as early electronic papers include a Gyricon bead reflective panel. This includes solid particles painted in two colors, and the particles are called Gyricon beads or the like.

An upper part and a lower part of each Gyricon bead are painted in white and black and charged negatively and positively, respectively. The bead is rotated by an electric charge of an electrode (a driver layer) that is contact with the bead, and thus the white and the black are switched from one to another. By controlling this for each pixel, an image can be displayed.

Color display can be performed by using beads of a combination of colors other than white and black or stacking color filter layers.

Alternatively, an electronic paper that includes an electrophoretic reflective panel may be used. In an electrophoretic panel used in an electronic paper or the like, micro capsules in which two colors of pigment particles charged positively and negatively and oil are enclosed are used as pixels.

By applying a voltage to upper and lower electrodes of a micro capsule, pigments of one of the colors move toward a display side. By performing this for each pixel, an image can be displayed.

A color visual content can be also displayed by using pigments of different colors such as RGB or by stacking color filter layers.

Alternatively, an electronic paper that includes an electronic liquid powder reflective panel may be adopted.

In an electronic liquid powder panel, unlike the electrophoretic panel, oil is not used. The electronic liquid powder panel has a structure in which two colors of electronic liquid powders charged positively and negatively move in the air.

In this structure, electronic liquid powders of one of the colors move toward a display side when a voltage is applied to upper and lower electrodes of a cell. By performing this for each pixel, an image can be displayed.

A color visual content can be also displayed by using electronic liquid powders of different colors such as RGB or by stacking color filter layers.

In addition to those described above, a visual output device in which a reflecting plate is disposed behind a liquid crystal panel having one of the various structures mentioned above (e.g., the electro-wetting panel, the electrochromic panel, the organic EL panel having transmittivity) may be used.

Alternatively, a liquid crystal on silicon (LCoS) projector may be used as the visual output device.

In a case of using the liquid crystal on silicon projector as the light stimulus device 10, light is reflected by a liquid crystal on silicon panel and then projected through lenses in a form of a visual content.

For example, in a three-chip LCoS projector, light applied from the light source 11 is split into red, green, and blue by a dichroic mirror, and visual contents of the colors made by the liquid crystal on silicon panel are combined and projected through a set of lenses.

For the color display, a method of turning on LEDs of different colors may be used, rather than splitting light with the dichroic mirror. Alternatively, the color may be limited to one, and a monochromic visual content may be projected.

As the reflective panel, an electro-wetting, electrochromic, Gyricon bead, electrophoretic, or electronic liquid powder panel may be used instead of the liquid crystal panel.

Alternatively, a digital light processing (DLP) projector may be used as the visual output device.

In a case of using the DLP projector as the visual output device, light is reflected by a DLP chip (also called digital micromirror device (DMD)) and then projected through lenses in a form of a visual content. The DLP chip is provided with the same number of minute mirrors as the number of pixels, and ON/OFF of display of each pixel can be controlled by changing a direction of a corresponding mirror.

For example, in an RGB color DLP projector, light applied from the light source 11 passes a color wheel rotating at high speed, is reflected by the DLP chip, and is projected in a form of a visual content. Images generated for the RGB colors are superimposed to be combined into a color visual content.

For the color display, high-speed switching of red, green, and blue LEDs may be used rather than the color wheel.

Alternatively, the color may be limited to one, and a monochromic visual content may be projected.

Alternatively, AR glasses that generate a visual content with a laser light source and a reflector may be used as the visual output device.

In a case of projecting a visual content directly onto a retina rather than observing a visual content projected onto a screen, a panel, or the like, a system including a laser light source can be used as the visual output device.

For example, there is a known technique of projecting an image onto a retina of a user by causing a MEMS mirror and a reflector to reflect light applied by RGB laser while adjusting an angle of the MEMS mirror at high speed.

In this case, the MEMS mirror and the reflector serve as the visual display unit 12 of a reflective type and the RGB laser serves as the light source 11.

Alternatively, the visual outputting device may project a visual content as the treatment light onto a medium having transmittivity. That is, the visual output device outputs a visual content that is projected onto a medium such as a partially reflective panel.

In this case, the light source 11 for the light stimulus is provided separately from a projector that projects a common visual content onto a screen or the like, and the user simultaneously sees the light from the light source 11 and the visual content projected by the projector.

As a specific mechanism, the projector or AR glasses mentioned above can be used.

The visual output device may project the visual content onto fine particles described later.

In a case of using blinking light as the treatment light, it is desirable to blink a visual content from a projecting machine such as a projector at the same timings as the treatment light so as to be synchronized with the blinking of the treatment light.

### (6-3) Modification of Fine Particle Discharge Device 30

Next, a modification of the fine particle discharge device 30 will be described.

The fine particle discharge device 30 of the system 1 can use not only the above-mentioned mist generated from warm water by the mist generator 32 but also various fine particles.

Candidates for types of the fine particles, means for generating the fine particles, and means for controlling a spatial disposition of the fine particles will be described below.

### (6-3-1) Solid Fine Particles

The fine particle discharge device 30 may discharge solid fine particles.

As a material of the solid fine particle, a plant-based material such as bark of Machilus thunbergii used to produce incense sticks or the like or tobacco leaves can be used. Under low temperature conditions, fine particles of ice can be also used. In a case of producing smoke by combustion, oils and fats that facilitate the combustion, adhesive for solidification, and the like can be used as additive.

Alternatively, the fine particle discharge device 30 may discharge dry powder that is used in a dry powder inhaler (DPI).

Specifically, sugar alcohol, polyol crystalline sugar, inorganic salt, organic salt, or the like can be used as a component of carrier particles. Examples of medicable active particles include the following.
steroid/bronchodilator, nitrate, antihistamine, anti-inflammatory agent, anticholinergic agent, leukotriene receptor antagonist, antiallergenic agent, antiemetic, hormonal agent, sympathomimetic agent, opioid, analgesic, immunomodulator, hypoglycemic agent, narcotic agonist or opiate antidote, phosphodiesterase inhibitor, antidepressant agent, serotonin agonist, serotonin antagonist, adrenaline agonist, adrenergic neuron blocker, benzodiazepine, antibiotic and antibacterial agents, antimicrobial agent, antiviral agent, vaccine, immunoglobulin, local anesthetic, antispasmodic, angiotensin converting enzyme inhibitor, angiotensin II receptor blocker, α-blocker, antiarrhythmic agent, anticoagulant, potassium channel regulator, hypocholesterolemic agent, diuretic agent, smoking cessation agent, bisphosphonate, dopamine agonist, nucleic acid medicine, and antipsychotic agent
Other pharmaceutically acceptable salts or any derivatives of those mentioned above

As a method of producing the fine particles, for example, the following methods can be used.
A method of mechanically pulverizing solid material
A method of discharging from a nozzle with pressurized air
A method of heating and burning a material and discharging the material in a form of smoke

In a case of generating the fine particles by combustion, the fine particles that are carbonized material are carried upward by an ascending air current caused by the heating.

### (6-3-2) Liquid Fine Particle

As a typical liquid fine particle, fog, which is the fine particles of warm water (water) mentioned above, can be used. As a device to generate the fog, the fine particle discharge device 30 may include the following devices instead of the mist generator 32.
An ultrasonic atomizer based on a principle of crushing a liquid by an ultrasonic transducer
A fog machine that discharges a heated liquid while cooling the liquid
A spraying device that jets pressurized air and a liquid simultaneously from a nozzle

Alternatively, in the fine particle discharge device 30, a method of atomizing water in the air or water available from around the fine particle discharge device 30 or water vapor in the air with dry ice may be used.

Likewise, in the fine particle discharge device 30, a method of atomizing a liquid such as alcohol or oil can be used.

Alternatively, the fine particle discharge device 30 may discharge mist with flavor using in an electronic cigarette or the like. In general, the electronic cigarette has a structure that heats and atomizes a liquid including a fragrance such as mint or coffee and supplies the liquid in a form of mist.

As a specific example of a component of a material of the fine particles, a liquid in which a fragrance is mixed into propylene glycol, glycerol, or the like can be used. As examples of the fragrance, a flavor of a fruit, a flavor of an herb, and a flavor of a tree can be used. Both a natural flavor substance and an artificial flavor substance can be used.

Alternatively, in the fine particle discharge device 30, a solution that includes a medicinal property used in a pressurized metered dose inhaler (PMDI) can be used. A specific component of the solution, various medicinal properties mentioned above dissolved in a solvent such as alcohols can be used. Alternatively, salts such as acetate and benzenesulfonate can be also used.

Alternatively, the fine particle discharge device 30 may discharge a mixture of the solid fine particle and the liquid fine particle mentioned above.

In the system 1, droplets discharged from a showerhead or a flowing liquid such as a jet of water may be disposed in an application range of the light source 11.

For example, by providing disposition means for disposing a moving liquid in a region to which the light source 11 applies the light, it is possible to alleviate the user's discomfort caused by the light stimulus. Here, the moving liquid refers to a liquid that moves to such a degree at which the user can visually recognize that the liquid is moving. The moving liquid preferably refers to a liquid that is configured to scatter the light applied from the light source 11 when the light is reflected on a surface of the liquid or inside the liquid or is transmitted by the liquid and further preferably to a liquid that is configured to vary the scattered light in direction and intensity with time. Examples of the disposition means include the following.
Disposing a rippling surface of the liquid between the eyes of the user and the light source 11
Moving the liquid in a space between the eyes of the user and the light source 11. Conceivable examples include a liquid falling down a waterfall and a liquid jetted from a fountain or the like.

The disposition means for disposing the moving liquid can be considered as means for giving movement to the liquid that is disposed in the region to which the light source 11 applies the light.

That is, in the present modification, between the light source 11 and the eyes of the user, a region in which the surface of the liquid ripples or the liquid moves in a space by falling down or being jetted is formed. With this configuration, the light applied from the light source 11 is scattered when passing the region, thus softening the light stimulus to the user. A specific example of the present modification will be described below.

### (Placing Light Stimulus Device 10 in Bathtub 100)

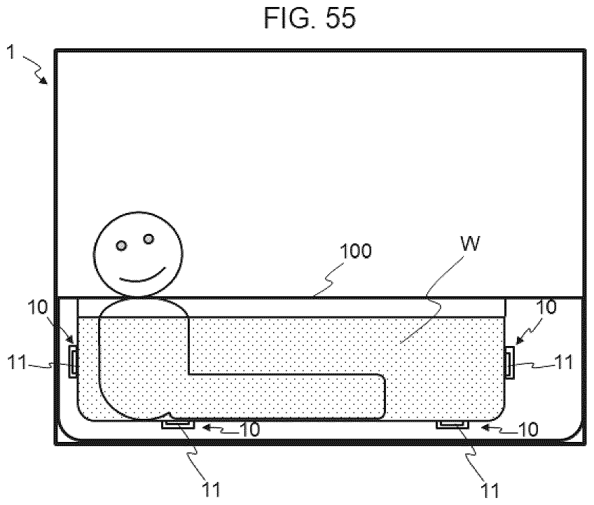

FIG. 55 is a schematic diagram of an example in which the light stimulus device 10 is placed in the bathtub 100. As illustrated in FIG. 55, the light stimulus device 10 is disposed in the bathtub 100 filled with water (warm water) as a liquid W. The user in the bathtub 100 ripples a surface of the water, and thus there is no need to provide an additional device to move the surface of the water. Note that the surface of the water may be rippled by a vibration generating device, such as a vibrating motor, placed in the bathtub 100. Ripples of the surface of the water alleviate the stimulus caused by the light applied from the light source 11 of the light stimulus device 10. The light stimulus device 10 is desirably embedded in the bathtub 100 so as not to form a projection in the bathtub 100. The same light stimulus device 10 may be placed in a bathtub for a footbath rather than a bathtub for full-body bathing.

The present modification dispenses with a need to place additional equipment such as the visual display unit 12 or the fine particle discharge device 30 in the bathtub 100. This therefore provides an advantage of easy preparation compared with the embodiment including the visual display unit 12 and the fine particle discharge device 30.

In addition, the method of disposing the rippling surface of the liquid between the light stimulus device 10 and the user does not need energy to generate fine particles from a liquid. This therefore provides an advantage of higher energy efficiency than a method of generating the fine particles.

The method of disposing the rippling surface of the liquid between the light stimulus device 10 and the user may be used in combination with the method using the visual display unit 12 or the method using the fine particle discharge device 30 mentioned above. For example, by using the fine particle discharge device 30 mentioned above to generate fine particles in a liquid with which the bathtub 100 is filled, it is possible to produce more complex reflection of light. This can enhance a visual presentation effect.

### (Placing Liquid Discharging Device)

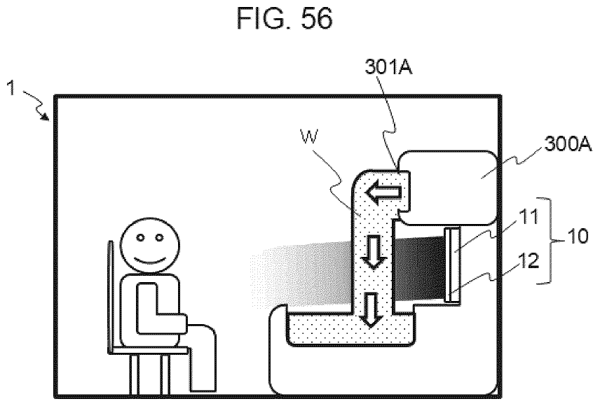

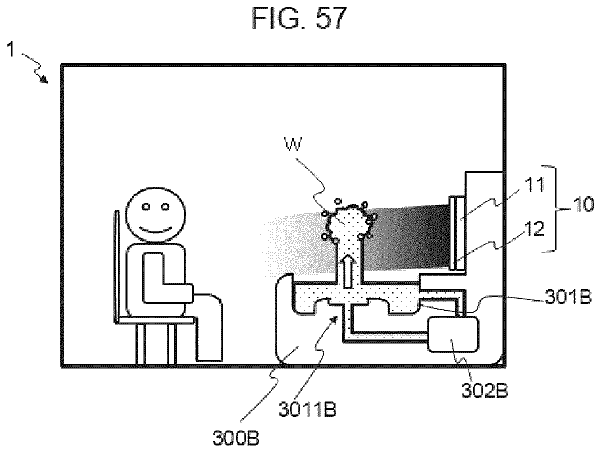

FIG. 56 and FIG. 57 are external views of an example of placing a liquid discharging device 300. The liquid discharging device 300 discharges a liquid to a region between the light stimulus device 10 and the eyes of the user, thus softening the light stimuli to the eyes of the user. The liquid discharging device 300 may discharge water as the liquid.

A liquid discharging device 300A illustrated in FIG. 56 causes the liquid W to fall down in a planar shape like a waterfall. The liquid discharging device 300A includes a discharge outlet 301A. The discharge outlet 301A is provided at a position higher than the light source 11. When the liquid W is supplied from a supply port not illustrated, the liquid discharging device 300A discharges the liquid W from the discharge outlet 301A in a direction indicated by arrows illustrated in FIG. 56. The liquid W falls down into a region between the light source 11 and the eyes of the user. The liquid discharging device 300A may circulate the liquid W to move the liquid W to the discharge outlet 301A again with a circulating device not illustrated.

A liquid discharging device 300B illustrated in FIG. 57 jets the liquid W in a three-dimensional manner like a fountain. The liquid discharging device 300B includes a liquid tank 301B and pressure generating means 302B. The liquid tank 301B includes a discharge outlet 3011B. The liquid tank 301B and the pressure generating means 302B are connected to each other. After the liquid W is supplied from a supply port not illustrated, the liquid tank 301B is filled with the liquid W. The liquid discharging device 300B causes the pressure generating means 302B to pressurize the liquid W with which the liquid tank 301B is filled. The pressurized liquid W is jetted from the discharge outlet 301 in a direction against gravity (e.g., a direction of an arrow illustrated in FIG. 57). As the pressure generating means 302B, for example, a pump, a motor, or a piezoelectric element can be used.

Alternatively, as the medium that alleviates the discomfort caused by the light stimulus, discharged “droplets” or a “liquid column” can be disposed between the light stimulus device 10 and the user. A specific example of this will be described below.

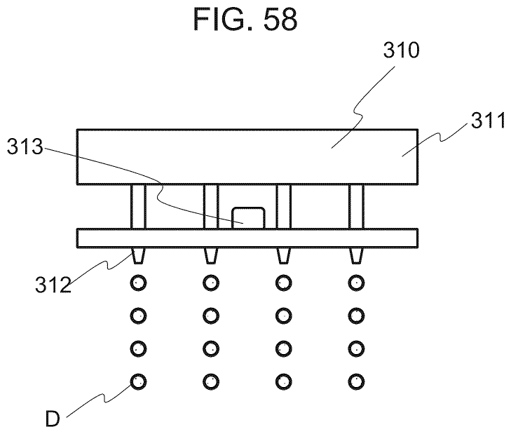

FIG. 58 is an external view of an example of a droplet generating device 310 that discharges droplets D. As illustrated in FIG. 58, the droplet generating device 310 includes a tank 311, a plurality of nozzles 312, and a vibrator 313. As the droplets D, for example, water droplets can be used. In the tank 311, water that is a material of the water droplets is enclosed. To the tank 311, the plurality of nozzles 312 are connected. When the vibrator 313 coupled to the nozzles 312 vibrates at a certain frequency, the nozzles 312 discharge the droplets in accordance with the vibration. As the vibrator 313, for example, an eccentric motor can be used. The droplet generating device 310 can discharge the droplets using an actuator that utilizes pneumatic pressure, hydraulic pressure, a piezoelectric element, or the like instead of the vibrator 313.

As illustrated in FIG. 58, by arranging the nozzles 312 in a row, it is possible to form an array of water droplets in a two-dimensional plane between the light stimulus device 10 and the user. Furthermore, by adding rows of nozzles in a direction that connects the light stimulus device 10 and the user, it is possible to form a three-dimensional array of water droplets. The droplet generating device 310 can continuously couple the discharged droplets into a liquid column shape by operating the vibrator 313 at high frequency.

Alternatively, in the present modification, by causing a predetermined projector to project a visual content onto the array of the water droplets discharged by the droplet generating device 310, it is possible to give the array of the droplets a function as a display as with the projection of a visual content onto fog, which is described in the embodiment described above.

### (6-3-3) Gaseous Fine Particles in Liquid

The fine particles used in the system 1 are not limited to fine particles floating in the air. Gaseous particles floating in a liquid may be used as the fine particles.

Specifically, a technique in which an aerator as the fine particle discharge device 30 is placed on a bottom surface of the bathtub 100 and gas bubbles are generated in accordance with a visual content projected onto a surface of water can be used. As a component of the gas bubbles, not only air but also carbon dioxide gas, nitrogen gas, and the like can be used.

As a method of generating such gas bubbles, a method of discharging gas taken in by a motor or piezo diaphragm pump into a liquid can be used.

As a method of generating fine gas bubbles such as microbubbles or nanobubbles, the following methods can be used.
A method using ultrasonication
A method using plasma called excimer laser ablation
A method using emulsification
An inkjet printing method

The microbubbles or nanobubbles discharged into the bathtub 100 in this manner undergo Brownian motion in hot water in the bathtub 100 and remain for a long time. Thus, the microbubbles or nanobubbles are suitable for producing a visual effect by irregular movement.

In the system 1, a liquid fine particle that is separated from a solvent (oils and fats in water) or a solid fine particle (metal powder, resin powder, etc.) may be used in the liquid. In a case of using magnetic particles or a liquid, behavior of the particles or the liquid can be controlled by using magnetism.

In addition, an aquarium including an aquatic animal, seaweed, plankton, or the like can be used as a device that scatters the treatment light.

### (6-4) Fine Particle Controlling Device 34

The system 1 may further include a fine particle controlling device 34 that controls a spatial disposition of the fine particles so as to hold the fine particles discharged by the fine particle discharge device 30 at an appropriate position.

As a method of controlling the spatial disposition of the fine particles, the fine particle controlling device 34 can use a technique of controlling movement of the fine particles or a technique of removing the fine particles. A specific example of this will be described below.

### (6-4-1) Control Means Using Structural Object

Movement of the fine particles can be inhibited by providing a physical barrier such as a partition.

For example, a structure in which the fine particles are accumulated inside a cup-shaped structural object can be used. Another structure that holds the fine particles at a specific location such as a box-shaped or tube-shaped structure can be used.

### (6-4-2) Control Means Using Gas Flow/Liquid Flow

As a method of running the fine particles discharged into the air into a desired position or removing the fine particles, means for generating a flow can be used.

For example, means for generating a gas flow, a fan can be used. The fan is desirably dampproof/dustproof so as to be used in an environment that is filled with the fine particles such as fog. Alternatively, the flow can be generated by generating positive pressure/negative pressure with a compressor, a vacuum pump, or the like.

As means for carrying the fine particles to a specific position, a technique of discharging a mass of gas can be used.

For example, a technique of discharging gas in a vortex ring shape can be used. As a component included in the gas to be discharged, the aroma components or medicable active particles mentioned above can be used.

In a case of using magnetic fine particles, an electromagnet, a ferrite magnet, or the like can be used as means for controlling a moving direction of a flow of the fine particles.

In a case of generating a flow of a liquid, a submersible motor or the like can be used. In a case of using a magnetic fluid as the liquid, an electromagnet, a ferrite magnet, or the like can be used as means for controlling the flow.

### (6-4-3) Means for Removing Fine Particles

It is also possible to control an area where the fine particles float by removing the fine particles and leaving the fine particles in an area where the fine particles are not removed. A specific removing method will be described below.

As a method of removing fine particles such as fog or ice fine particles, removing by heating with application of infrared rays can be used. As an application source of the infrared rays, a carbon fiber heater, an infrared semiconductor laser, or the like can be used.

It is also possible to remove gaseous fine particles in a liquid by applying infrared rays to the gaseous fine particles to heat the gas, causing the babbles to expand and rise up to a surface of the liquid.

As a method of cutting off a flow of solid fine particles, there is a method using an electric discharge in a gas. For example, a mechanism in which high voltage is applied to a needle electrode and a trigger electrode and electrons are discharged from the needle electrode to generate an ionic wind (a flow of ionized dust and gas molecules) can be used.

The ionic wind can be also used in a case of causing liquid fine particles to fall down in a form of water droplets through condensation. For example, it is conceivable to apply the ionic wind to fog, thus causing water droplets to fall to stage a rainy appearance.

As means for removing gas bubbles in a liquid, in addition to the use of infrared rays mentioned above, a method of removing gas bubbles by expanding the gas bubbles through heating or using negative pressure and a method of removing gas bubbles by contracting the gas bubbles through pressurization to make the gas bubbles invisible or by dissolving the gas bubbles in the liquid can be used.

### (6-5) Fine Particle Recognition Device 35

The system 1 may further include a fine particle recognition device 35 that recognizes a disposition state of the fine particles so as to grasp the disposition state of the fine particles discharged by the fine particle discharge device 30 to hold the fine particles at an appropriate position.

The fine particle recognition device 35 includes an image sensor such as a CMOS or a CCD to observe the disposition state of the fine particles.

The fine particle recognition device 35 may be, for example, a USB camera with approximately hundreds of thousands to ten million pixels.

The fine particle recognition device 35 records an image before the fine particles are generated as an initial state, compares the initial state with an image after the fine particles are generated, and recognizes a range where the fine particles are present from differences in RGB values for each pixel.

Alternatively, the fine particle recognition device 35 may recognize an area where fog is present (or an area where fog is not present) with an infrared distance sensor, a photo interrupter sensor, an ultrasonic motion sensor, or the like.

In these cases, positions at which the sensors are provided may be on a device used in the light treatment and may be any position in a room such as a lighting instrument or a window.

The fine particle recognition device 35 may use a camera that is provided in a general-purpose indoor robot.

### (6-6) Modification of Antifog Means

As means for removing fogginess or contamination in the visual output device, spraying a liquid or gas can be used. For example, a mechanism of spraying cleaning solution can be used.

In addition, a mechanism of blowing dry air or hot air, a mechanism of suctioning contamination with negative pressure, a mechanism of physically removing water droplets or dust, or the like by a wiper can be used.

### (6-7) Modification of Voice Recognition Device 50

For the voice recognition device, a tablet computer, a smartphone, or a PC that is operated with a touch panel or the like may be used instead of the smart speaker 61.

Alternatively, a device that recognizes a position or motion of the user may be used as a trigger for starting, finishing, and the like the therapy.

For example, there is known an IoT device that detects an action of the user with an image sensor such as a CMOS, an ultrasonic motion sensor, an infrared sensor, a motion detection sensor, or the like, thus operating another device such as a lighting instrument. These devices may be used for starting and finishing of the light treatment according to the present invention. For example, it is possible to start the treatment when detecting that the user has risen.

### (6-8) Modification of Treatment Content

Next, a modification of the treatment content will be described.

### (6-8-1) Content for VR Device or Three-Dimensional Display

As the treatment content, content for a VR device or a three-dimensional display may be used. In such a case, the content generating module 2032 generates left-eye and right-eye image data items separately. By the content outputting module 2033 displaying the respective images on a display of the VR device or the three-dimensional display as the visual output device, the user can use a three-dimensional visual content as content.

In particular, as a visual content highly effective in urging the user to watch carefully, a “flame,” a “painting,” a “photograph of a person,” a “photograph of the user's album,” a “stock chart,” a “pattern,” and the like can be cited in addition to the natural scene.

### (6-8-2) Game Content

As the treatment content, content such as a video game may be used. As equipment to display a visual content of a game, a liquid crystal display, an organic EL display, or the like can be used.

### (6-8-3) Modification of Method of Generating Treatment Content

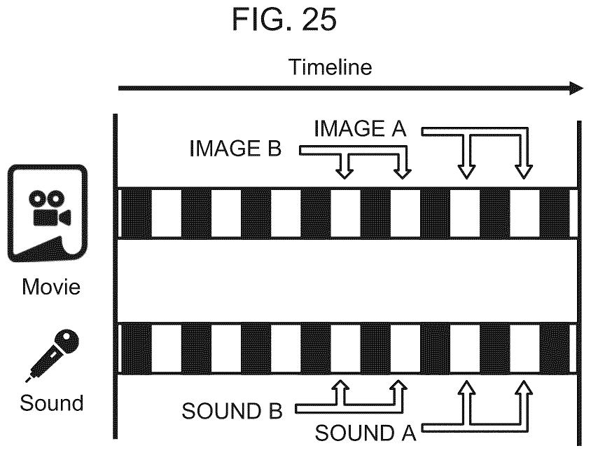

FIG. 25 is a diagram illustrating a method of generating the treatment content according to a modification.

As illustrated in FIG. 25, in the method of generating the treatment content according to the modification, the content generating module 2032 generates the treatment content by inserting an image and a voice.

That is, the content generating module 2032 generates the treatment content by inserting prepared image files and voice files into ON parts in template data in which ON/OFF timings are disposed at certain intervals.

By adopting this generating method, for example, it is possible to playback photographs in an album of the user at random. The treatment content obtained by this generating method has a disadvantage in that the treatment content lacks change compared with a video but has an advantage from a viewpoint of a reduction in file size and consistency of its stimulus because of its simplified composition.

The content generating module 2032 may also convert a color tone of visual data on the treatment content to apply light having a wavelength that fits a purpose.

The content generating module 2032 performs a process of converting a color tone of an image or a video to increase an intensity of the image or the video at a specific wavelength so as to enhance effects of the light treatment.

For example, in a case of intending to generate the treatment content consisting of red light, a process of converting colors of green and blue parts into red can be used. More specifically, a process of depicting autumn leaves by converting a color of leaves of a tree from green to red, depicting a sunset glow by converting a blue sky into a red sky, or the like can be used.

In the system 1, a visual presentation may be added to the treatment content by using the discharged fine particles. For example, by using the fine particles discharged in front of the display surface of the visual output device, it is possible to express clouds, smoke, or the like. At this time, the content generating module 2032 causes the fine particle discharge module to accumulate the fine particles such as fog forward of the visual output device.

That is, in a case where a photograph of the sky with clouds is output from the visual output device, the content generating module 2032 grasps a relative spatial region in the photograph where the clouds are present. Then, the fine particle discharge device 30, the fine particle controlling device 34, and the fine particle recognition device 35 are caused to execute a control program to perform control of discharging and disposing the fine particles such that the fine particles float in the grasped spatial region.

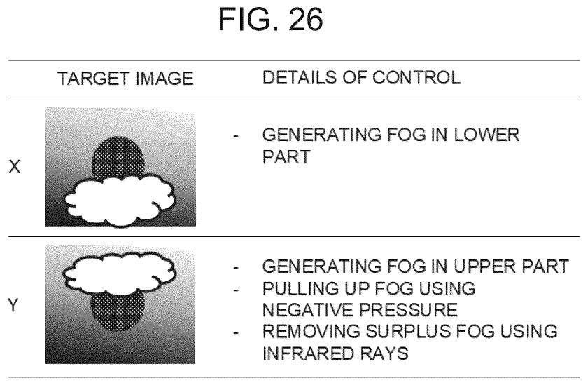

FIG. 26 is a diagram illustrating an example of the visual presentation using the fine particles for the treatment content.

As illustrated in FIG. 26, in a case where an image to be displayed to the user (a target image) is an image X, the content generating module 2032 causes the fine particle discharge module to execute control pertaining to discharging the fine particles such that the fine particles float in a lower part of the image. Such control of disposing the fine particles will be described below in detail.

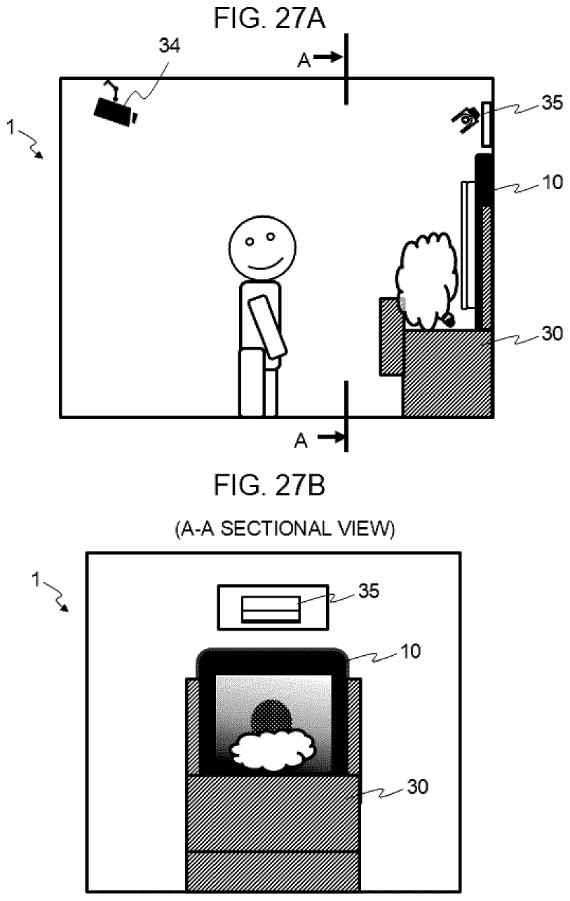

FIG. 27A is a diagram illustrating a state of the system 1 corresponding to the image X in FIG. 26. FIG. 27B is a diagram illustrating a state of the system 1 corresponding to the image X in FIG. 26.

As illustrated in FIG. 27A and FIG. 27B, in a case where the clouds are positioned in the lower part, the fine particles are generated and accumulated in the lower part. In a case where the fine particles are fog, the fog tends to move in a gravity direction. Thus, by providing a member that serves as a receiving pan, it is possible to accumulate the fog in a target region without gas flow control, removal of the fog, or the like.

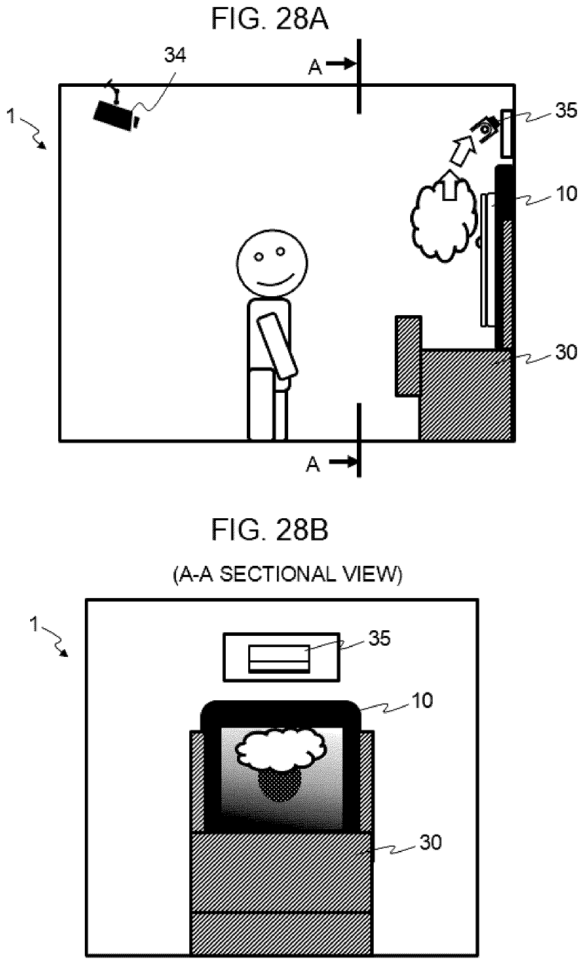

FIG. 28A is a diagram illustrating a state of the system 1 corresponding to an image Y in FIG. 26. FIG. 28B is a diagram illustrating a state of the system 1 corresponding to an image Y in FIG. 26.

As illustrated in FIG. 28A and FIG. 28B, in a case where the clouds are positioned in an upper part, the fine particles are also generated in the upper part. In a case where the fine particles are fog, by causing the fog to flow upward by a suction fan so as not to cause the fog to flow down and removing the fog flowing outside the screen using infrared rays, it is possible to express “clouds floating at a certain position.”

In these examples, it is desirable to display a background image in a variable manner on the display or the like. However, the sun or the moon may be expressed by placing the light source 11 in a spherical shape in front of a fixed background such as a painting, or a scene may be expressed using a three-dimensional model such as a mountain or a tree.

An infrared-ray irradiator used for controlling a shape of the fog may take not only a role of removing the fog but also a role of a heater that warms a body of the user.

In the system 1, a visual presentation may be added to the treatment content by using the fine particles in a liquid.

For example, also in a case where the fine particles such as air are accumulated in the liquid, a position at which the fine particles are intended to be accumulated may be specified using the target image or the like, and the fine particles may be generated, moved, removed, and the like such that the fine particles are accumulated at the specified position, as in the process mentioned above.

The position at which the fine particles are to be accumulated may be read from the target image, or the user may newly specify the position at which the fine particles are to be accumulated. For example, the user may use a drawing function of software to add a drawing of clouds on image content to be displayed, and a control program that generates and removes fog in disposition corresponding to a position of the added clouds.

The target image may be either a 2D image or a 3D image. In a case where the control is performed in accordance with a 3D image, it is necessary to control of generating, moving, and removing the fine particles more complexly. Therefore, it is desirable to provide a plurality of means for controlling spatial disposition of the fine particles or provide an actuator that moves the means for controlling the spatial disposition of the fine particles.

### (6-9) Modification of User Analysis Device 40

Next, a modification of the user analysis device 40 will be described. Here, a configuration in which various wearable devices are used as the user analysis device 40 will be described.

In a case where the user wears a wearable device, by using the wearable device as the user analysis device 40, control such as suspending the therapy when an anomaly in the user is detected may be performed based on biological information obtained from the wearable device.

### (6-9-1) Watch-Type Wearable Device

As the user analysis device 40, a smartwatch that is capable of obtaining biological information on the user can be adopted.

In this case, the user analysis device 40 can measure, for example, a heart rate, a respiratory rate, a blood oxygenation level, a blood pressure, a body temperature, and the like and can estimate a blood volume.

In particular, the heart rate and the respiratory rate serve as indices of a state of mind, a state of concentration, and the like of the user and thus can be used to grasp compatibility between the user and the treatment content. The heart rate and the respiratory rate can be used for a safety check on whether there is no anomaly in respiration and heartbeat of the user.

Since many smartwatches have a function of operating an external device, the smartwatch can be used as a terminal to operate starting and finishing the treatment.

Furthermore, in a case of adopting, as the user analysis device 40, a smartwatch that includes a CMOS sensor, a CCD, a spectroscope, a hyperspectral camera, or the like, it is possible to obtain a log of wavelengths of light to which the user is exposed during the daytime. Thus, it is possible to grasp a tendency adapted to the user's lifestyle such as excessive exposure to blue light or lack of light exposure during the daytime. Therefore, the log of wavelengths of light to which the user is exposed during the daytime can be used at a time of recommending treatment suitable for the user, or the like.

### (6-9-2) Glasses-Type/Contact-Lens-Type Wearable Device

As the user analysis device 40, a glasses-type/contact-lens-type wearable device can be adopted.

For example, as the user analysis device 40, a device that has a shape like glasses and can track, with a camera or the like provided in the device, a scene that the user is watching can be adopted. By using this device, it is possible to obtain, from the user analysis device 40, information on whether the user can directly look at the light actually, an actual intensity of the light input into the user, health conditions of the eyes of the user, and the like during the treatment by the system 1.

Alternatively, for example, a device that can grasp an activity of eyes such as blinking using a camera, an infrared sensor, or an electrooculogram (EOG) sensor may be adopted as the user analysis device 40. In this case, it is possible to grasp whether the user can concentrate on observing the light during the light treatment by the system 1.

Alternatively, a wearable device having a function of inspecting areas of retinae can be adopted as the user analysis device 40. In this case, retinal conditions can be inspected on an area basis. The same device can be used as a device that applies red light, and thus it is possible to perform a red light therapy on only an area where the retinal conditions deteriorate.

Alternatively, in a case of using, as the user analysis device 40, smart glasses that include a CMOS sensor, a CCD, a spectroscope, a hyperspectral camera, or the like, it is possible to obtain a log of wavelengths of light to which the user is exposed during the daytime. In this configuration, the smart glasses can measure a log of wavelengths of light to which the user is exposed during the daytime at a position closer to the eyes than the user analysis device 40 of a smartwatch type, and thus it is possible to measure wavelengths of light that enter the eyes of the user more accurately.

Alternatively, the contact-lens-type wearable device may be adopted as the user analysis device 40. In this case, it is possible to measure a concentration of melatonin, interleukin-6, and the like included in a tear. Alternatively, in a case where the system 1 is used for adjusting a circadian rhythm or performing treatment for seasonal affective disorder (SAD), it is possible to use a phase of the circadian rhythm of the user estimated from a result of the measurement as reference data for determining a timing of the treatment.

In addition to the above, various wearable devices such as an earphone type, a headphone type, and a clothing type can be used as the user analysis device 40.

Alternatively, various biological information sensors provided in bedding, a toilet, the bathtub 100, or the like can be used as the user analysis device 40.

### (6-10) Other Devices

Next, other devices that can be added to the system 1 will be described.

### (6-10-1) Shading Device

The system 1 may further include a shading device.

In treatment that requires a timing at which no light enters eyes, such as the light stimulus with the gamma frequency, for example, light entering through a window of the bathroom causes noise, which may have an adverse influence on effects of the treatment. Therefore, it may be necessary to use the shading device to prevent the optical noise from penetrating.

As the shading device, a window that can be remotely operated to control a transmittance of light using an electrochromic system. In this window, when a voltage is applied to its transparent electrodes, an oxidation state of its color developing layer changes, which changes its color, thereby shielding external light.

As the shading device, a shutter, a blind, or the like having an automatic opening and closing function may be used.

### (6-10-2) Sound Insulation Device

The system 1 may further include a sound insulation device.

For example, in a case where the user receives the sound stimulus from a wearable terminal used in a form of an earphone or a headphones, when a wearable terminal having a noise canceling function is adopted, the wearable terminal also functions as the sound insulation device.

In a case where the user receives the sound stimulus from a distance, when the speaker 63 having a spatial noise canceling function is adopted, the speaker 63 also functions as the sound insulation device. In this case, for example, the sound insulation device estimates sound noise that reaches the ears of the user from information input to a sound sensor and positional information on the user and generates a sound that has the same amplitude as the sound noise and an opposite phase to the sound noise to cancel the noise.

Alternatively, a structural object made of soundproof material or the like may be adopted as the sound insulation device.

### (6-10-3) Indoor Robot

The system 1 may further include an indoor robot.

In a house or an office, a robot that supports safety management and communication may be used in the system 1. Various sensing functions provided in these robots may be used for sensing of conditions of the user performed in the present invention.

For example, the indoor robot monitors the user under the treatment for occurrence of a fall, an anomaly in respiration, or the like, and when an anomaly occurs, the indoor robot may set off an alarm.

In addition, these robots may perform monitoring of the spatial disposition of the fine particles mentioned above, observation of the orientation of the face of the user and open/close of the eyes of the user, operation of other devices, and the like.

### (7) Other Usages

Next, a configuration for using the system 1 in a location other than the bathroom will be described.

### (7-1) Use in Toilet

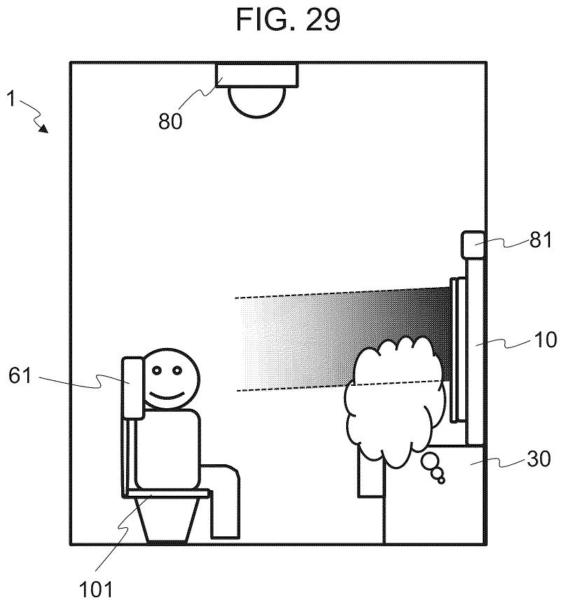

FIG. 29 is an external view of a case where the system 1 is used in a toilet.

As illustrated in FIG. 29, the system has the same configuration as in use in the bathroom mentioned above also in a case where the system 1 is used in the toilet. Then, other devices are operated by instructions with an utterance from the user to the smart speaker 61. A specific process flow at this time is the same as the process described above.

Here, the smart speaker 61 is desirably disposed at an upper part of a backrest of a toilet bowl because effects of the smart speaker 61 are maximized when the smart speaker 61 is placed at a position close to the ears of the user, as in the bathroom.

As the fine particle discharge device 30, it is desirable to use an ultrasonic atomizer, which can gently generate fog, because leakage of water is more of a problem in the toilet than in the bathroom.

The toilet is used more frequently than the bathroom, and the toilet is used many times a day, and thus the details of the treatment protocol may be changed every time. For example, it is conceivable to apply, to the user, a light stimulus of a wavelength that follows changes in wavelength of sunlight by time of day. In addition, as the displayed visual content, it is desirable to display a scene that fits a time of day.

In a case where the system 1 is used in the toilet, a biological information sensor placed on a toilet bowl that is to come into contact with the user may be used as the user analysis device 40.

For example, a technique of monitoring conditions of the user with a blood concentration sensor, a pressure sensor, an electrostatic sensor, or the like placed on a toilet seat can be used. By using these techniques, it is possible to obtain health data on the user under the treatment and to detect an anomaly in the user and set off an alarm.

A duration of using the toilet differs every time, and the user may input a voice command for suspension to finish the treatment before a duration set in the treatment protocol expires. The treatment can be finished by detecting that the user has stood up, with a motion sensor or a contact sensor.

Note that the system described above using the toilet may be used not only for the gamma stimulus therapy but also for prevention of or a therapy for other types of disorders in health conditions such as seasonal affective disorder, winter depression, jet lag, retinopathy, pain control, and wellness improvement.

### (7-2) Use in Amusement Facility Such as Disco or Karaoke Room

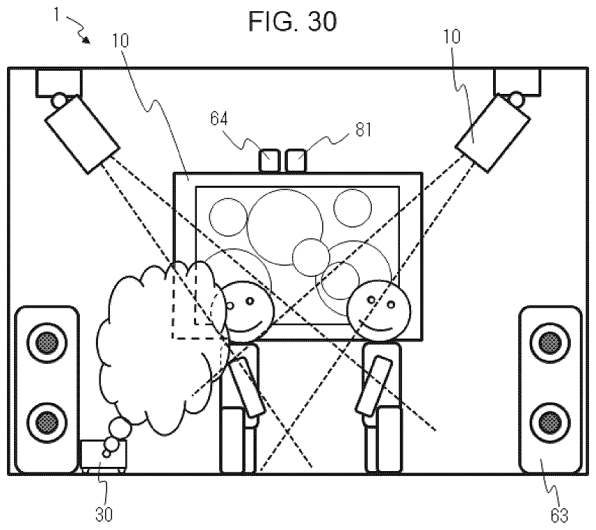

FIG. 30 is an external view of a case where the system 1 is used in an amusement facility such as a disco or karaoke room.

In a disco, in general, a plurality of stage lighting instruments and speakers 63 are used in addition to a display.

In this usage example, the display and the stage lighting instruments output the light stimuli in synchronization with a timing of a period of the gamma frequency. Whether there is any shift between the light stimuli and the sound stimuli generated by the devices is checked by observing waveforms from the optical sensor 81 and the microphone (sound sensor) 64 placed on the display or the like, the content generating unit of the controller 20 revises the shift by applying an offset.

In a case where the system 1 is used in a disco, a fog machine that is used as a stage device or the like can be used as a fine particle generating device.

A fog machine generates a large amount of mist by heating a liquid (a mixture of ethylene glycol and water, etc.) supplied from a tank and discharging the heated liquid while cooling the liquid. Therefore, the fog machine is suitable for use in a large space such as a disco.

Constantly generating the light stimulus and the sound stimulus with the gamma frequency may cause the user's fatigue. Thus, a timing of generating the gamma stimulus may be provided in intermissions of playing pieces of music and displaying visual contents as in a usual disco.

As a method of aligning timings of generating the stimuli, a method of time stamping data and synchronizing timings of outputs by the plurality of devices with one another can be used. For example, by the content generating unit time stamping the visual content and the sound content, it is possible to output the light stimulus and the sound stimulus from the plurality of devices in synchronization with one another.

### (7-3) Use in Bedroom

The system 1 can be used for adjusting a circadian rhythm aimed at a therapy for or prevention of seasonal affective disorder (SAD) and winter depression. Many SAD patients have a problem of a decline in energy due to an insufficient amount of light, and a solution to the problem is application of light in early morning.

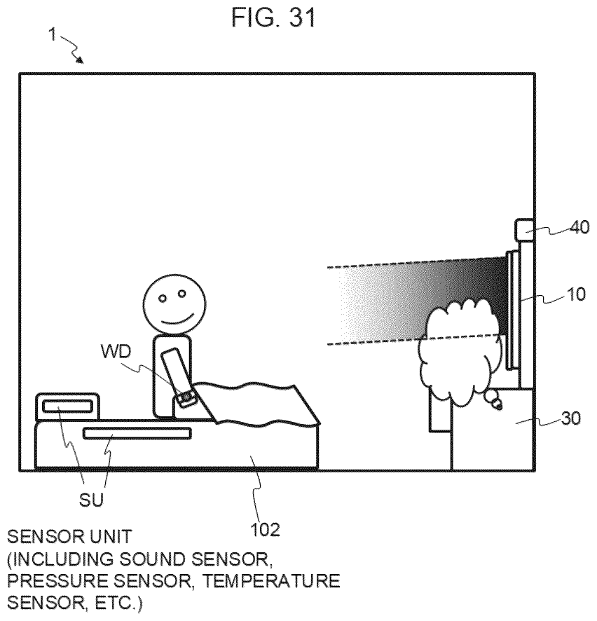

FIG. 31 is an external view of a case where the system 1 is used in a bedroom.

As illustrated in FIG. 31, when used for adjusting a circadian rhythm, the system 1 is provided in the bedroom.

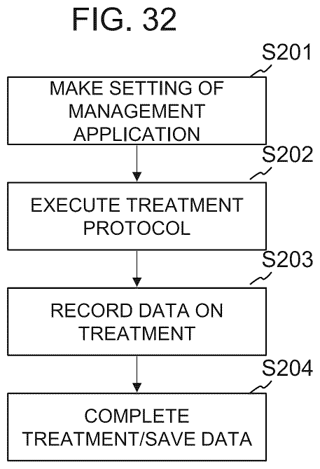

FIG. 32 is a diagram illustrating a process at a time when the system 1 is applied to adjustment of a circadian rhythm.

In the adjustment of a circadian rhythm, first, a setting of the management application for the light stimulus treatment is made (step S201).

Specifically, the user logs in to the management application on the user terminal 5 and sets a target wake-up time and details of the treatment protocol (the visual content, the amount of the mist, the duration of the treatment, etc.).

Here, in the adjustment of a circadian rhythm, it is desirable to apply, as the light stimulus, light that has an amount of light of 1000 to 5000 Lux and includes a wavelength around 480 nm, including 480 nm itself, which is used in typical light therapy. To obtain a sufficient amount of light, a high-luminance liquid crystal display is suitable as the visual display unit 12.

In the adjustment of a circadian rhythm, by selecting, for example, a visual content of a sunrise as the treatment content, it is possible to stage a morning atmosphere. As the fine particles generated forward of the display, it is desirable to use fog generated by an ultrasonic atomizer.

The management application sends the set treatment protocol to the controller 20. These items of data are saved in a memory of the controller 20, and preparation for starting the treatment is made.

After step S201, the treatment protocol is executed (step S202).

Specifically, when the set wake-up time comes, the content outputting unit drives the visual output device to generate the light stimulus in accordance with the protocol. The light stimulus itself has an awakening function and facilitates the user's wakefulness.

In order to further facilitate the user's wakefulness, the sound stimulus device 60 may output sound, and gas may be discharged in a vortex ring shape. For example, a vortex ring of air with a flavor of citrus or the like, which accelerates the wakefulness, may be supplied to a vicinity of a face of the user.

After step S202, the system 1 records data on the treatment (step S203).

Specifically, behavior of the user is analyzed by the user analysis device 40 to check whether the treatment is performed normally because the user has just woken up, and the user may close the user's eyes. For example, the user analysis device 40 uses a camera to record the orientation of the face and the movement of the eyes of the user, an amount of light that can be applied when the eyes of the user are opened, and a duration of the application. In addition, a degree of concentration of the user is evaluated by measuring the number of blinks per a certain period of time.

Here, in a case where the user wears a wearable device, data obtained from the wearable device, such as a heart rate and a blood oxygen saturation level, may be obtained simultaneously. By grasping, from data such as a heart rate, a stress that the user feels during the treatment and accumulating the stress as data, it is possible to grasp a treatment protocol that is suitable for the user. In addition, control such as suspending the therapy when an anomaly in the user such as a rapid increase in heart rate is detected can be performed.

After step S203, the system 1 completes the treatment and saves data (step S204).

Specifically, the controller 20 finishes driving the devices when the duration of the treatment set in the protocol expires. A log of the treatment is sent from the controller 20 to the management application, and the sent data is saved in the cloud server 82 via the network.

Such use of the system 1 in the bedroom can be used not only as measures against SAD and winter depression but also for treatment for other types of disorders in health conditions such as dementia measures, jet lag, retinopathy, pain control, and wellness improvement.

Next, a method of monitoring effects of the light stimulus treatment will be described.

In order to grasp effects of the treatment described above, measurement of a body temperature, a pulse, a heartbeat, a blood pressure, a breath sound, and the like with a watch-type wearable device as the user analysis device 40 or a sensor built in bedding can be used. By grasping timings of falling asleep and waking up from these items of data, it is possible to estimate a phase of a circadian rhythm of the user.

These types of data from the sensing device can be referred to from the management application installed on the user terminal 5. By grasping whether a rhythm of falling asleep and waking up of the user approaches its target, it is possible to monitor the effects of the treatment. The timings of falling asleep and waking up vary day by day, it is desirable to collect data spanning from approximately one week to approximately one month and evaluate its average value and median.

In a case where a contact-lens-type wearable device is used as the user analysis device 40, it is also possible to measure concentrations of cortisol, interleukin-6, and the like in a lacrimal fluid. This is because the phase of a circadian rhythm is clinically determined by measuring changes with time in melatonin concentration and cortisol concentration and thus can be grasped by inspecting saliva, blood, or the like of the user.

In addition, measurement methods using electrodes attached to a body, such as electroencephalography (EEG), electrooculography (EOG), electromyography (EMG), and electrocardiography (ECG), can be used as a method of accurately monitoring sleep. However, these measurement methods involve laborious tasks such as extracting a body fluid and attaching the electrodes and thus are difficult to use routinely. Thus, sensing using a wearable device is desirable.

In a case of a disease in which a plurality of factors are involved, such as a disruption of serotonin in addition to a disruption of the circadian rhythm, as in a case of SAD, there is a possibility that its symptoms do not improve even when a phase shift of the circadian rhythm is eliminated.

In contrast, by using the system 1, it is possible to report the elimination of the phase shift of the circadian rhythm using data that is accumulated in a form of the log of the treatment, and thus it is possible to provide a medical professional such as a doctor with information for considering another therapeutic approach such as medication.

The system 1 can be also used for prevention of or a therapy for jet lag, delayed sleep phase syndrome (DSPS), advanced sleep phase syndrome (ASPS), or the like. In a case where treatment for ASPS is performed, it is desirable to perform the light stimulus treatment in the evening or nighttime, rather than in the early morning.

(7-4) Use of System 1 for Improving Cognitive Performance, Etc. Using Red Light

As another utilization of the system 1, photobiomodulation therapy (PBMT) and improvement of cognitive performance using red light and infrared rays are cited.

PBMT is a therapeutic method aimed particularly at age control of a retina and a cure for retinal damage, and at prevention of or therapy for diabetic retinopathy (DR) or age-related macular degeneration (AMD).

In the light stimulus treatment with red light, as in FIG. 32, a setting of the management application for the light stimulus treatment is first made.

The user logs in to the management application on the user terminal 5 and sets details of the treatment protocol (the visual content, the amount of the mist, the duration of the treatment, etc.).

In this case, light of a wavelength in a red region (620 to 760 nm), particularly a wavelength of 670 nm, is applied as the light stimulus, and it is desirable to use a visual content predominantly featuring red color such as a sunrise or a sunset glow, as a visual content that is scattered by the fine particles and input into the user.

In addition, it is desirable to use light including a wavelength in near infrared region (780 to 825 nm), particularly a wavelength of 810 nm. As a mechanism in which a high-luminance red LEDs can be used, a liquid crystal display is suitably used.

As specific treatment content, it is desirable to display a visual content that is highly compatible with artificial clouds formed by the fine particles, such as a sunset cloud sea.

In addition, it is known that application of red light (particularly, light of a wavelength of 630 nm) in the morning improves cognitive ability upon waking up, thus exerting an effect on sleep inertia and the like. Thus, visual content that imitates a sunrise may be displayed in the early morning.

As the fine particles generated forward of the display, it is desirable to use fog generated by an ultrasonic atomizer.

The management application sends the set treatment protocol to the controller 20. These items of data are saved in a memory of the controller 20, and preparation for starting the treatment is made.

Next, as in FIG. 32 mentioned above, the system 1 executes the treatment protocol, records data on the treatment, completes the treatment, and saves data in the cloud server 82.

Here, PBMT exerts effects in not only improving retinal conditions of an eye but also improving body conditions such as muscle damage and thus may be used in a bathroom to improve overall conditions of a body including an eye. In this case, the bathroom described in the embodiment about the treatment for dementia can be used.

In order to grasp effects of the treatment with red light described above, an eyesight test using an application in a smartphone or the like or retinal inspection using a glasses-type wearable device can be used.

By undergoing, as an inspection in a medical institution, an eyesight test, a funduscopy, a retinal image diagnosis, contrast sensitivity (CCS) inspection, or the like, it is possible to obtain more accurate diagnosis results.

(7-5) Utilization when Present Invention is Applied to Pain Control with Green Light

As another utilization of the system 1, treatment with green light aimed at pain control of episodic migraine (EM), chronic migraine (CM), postoperative pain, or the like is cited. This description will be given of a configuration for using the system 1 on a bed in a sickroom as in FIG. 31, assuming a user who is recuperating after surgery, or the like.

In the light stimulus treatment with green light, as in FIG. 32, a setting of the management application for the light stimulus treatment is first made.

The user logs in to the management application on the user terminal 5 and sets details of the treatment protocol (the visual content, the amount of the mist, the duration of the treatment, etc.).

In this case, light having a wavelength in a green region (490 to 550 nm), particularly a wavelength around 525 nm, including 525 nm itself, is applied as the light stimulus. It is desirable to apply light having a luminance as low as 300 Lux or less because strong light causes pain. As the display, an organic EL display or the like as well as a liquid crystal display is suitable.

As specific treatment content, it is desirable to use a visual content predominantly featuring green color, such as forest. As the fine particles generated forward of the display, it is desirable to use fog generated by an ultrasonic atomizer to create an appearance such as a “foggy forest.”

It is possible to further enhance a pain control effect by using, as a component of the fine particles, a flavor of lavender, a terpenoid compound such as linalool, or phenols such as methyl salicylate, which have the pain control effect.

The management application sends the set treatment protocol to the controller 20. These items of data are saved in a memory of the controller 20, and preparation for starting the treatment is made.

Next, as in FIG. 32 mentioned above, the system 1 executes the treatment protocol, records data on the treatment, completes the treatment, and saves data in the cloud server 82. The user may operate the user terminal 5. However, it is desirable that the treatment is started with a voice or a motion so as to avoid suffering pain from a contact stimulus as much as possible.

In order to grasp effects of the treatment with green light described above, measurement of a body temperature, a pulse, a heartbeat, a blood pressure, a breath sound, and the like with a sensor built in bedding can be used. From these items of data, it is possible to estimate a stress level and a sleep quality of the user, thus judging whether the treatment exerts the effects. The measurement may be performed using a wearable device. However, it is desirable to use a device that is built in bedding with consideration given to the user's stress due to wearing the device.

In addition, as an inspection in a medical institution, a medical interview about a pain level, concentrations of serotonin and noradrenaline in blood or saliva, recording brain waves by EEG, and the like can be used.

### (7-6) Use of System 1 for Improvement in Health Conditions or Mood

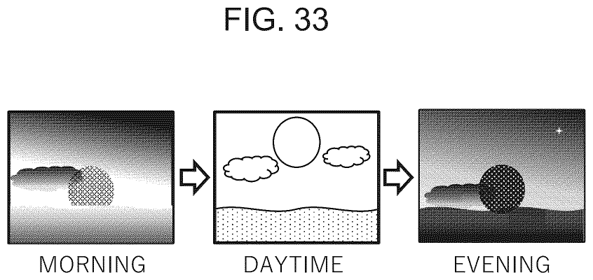

As another utilization of the system 1, treatment for improvement in health conditions or a mood is cited. In this case, the system 1 uses the light stimulus that changes in wavelength in chronological order. FIG. 33 is a diagram illustrating an example of a visual content that changes in wavelength in chronological order.

As illustrated in FIG. 33, as a visual content that changes in wavelength in chronological order, a visual content that imitates changes of outdoor sunlight is used. As illustrated in the figure, the wavelength of the light in the visual content changes with time.

In this usage method, it is desirable to display an outdoor scene corresponding to a time of day so as to display changes in outdoor scene from a sunrise to a sunset. The scene may be a scene of an area where the user lives, and scenes from around the world can be reproduced and displayed with chronological changes taken into account.

In a case where the light stimulus is controlled in the system 1 with the display and the fine particles combined, it is desirable for the display to display a visual content that is highly compatible with an artificial cloud created by the fine particles, such as a combination of the sea and sky.

The system 1 may adopt, instead of the visual output device, a simple configuration such that the fine particles imitating clouds are discharged in front of a spherical lighting instrument or the like that imitates the sun.

(7-6) System 1 for Relaxation or Facilitation of Falling Asleep with Light Blinking Over Long Cycle

As another utilization of the system 1, using the system 1 to apply light blinking over a long cycle that is aimed at a relaxing effect is cited.

Light slowly blinking has a relaxing effect, thus being suitable for sleep induction and the like. Thus, for example, it is possible to facilitate falling asleep by displaying an image of a night view or the like on the display as a pattern in a bedroom while blinking the image.

In addition, by blinking the green light for pain control mentioned above, it is possible to provide the user with the pain control effect by the green light and the relaxing effect by the blinking.

In any of the cases, a period of the blinking is desirably two seconds or more.

### (7-7) System Recommending Appropriate Treatment

The embodiment for prevention of or a therapy for a specific disease or ill health has been described thus far. However, there is a case where the user is unsure about “what disease I should take measures against” to begin with. In particular, there is no subjective symptom in a prevention stage, and thus it is desirable to detect a sign by an inspection device and recommend appropriate treatment.

To support a plurality of types of treatment, the treatment device is also required to have a configuration that supports a plurality of types of treatment. A process of inferring the appropriate treatment and recommending the appropriate treatment to the user will be described below.

First, the user makes a setting such that an application on the inspection device (a wearable device, a sensing device, a smartphone, etc.) sends data to a treatment management application. The treatment management application analyzes the user's conditions based on the received data and infers necessary treatment.

For example, in a case where it is inferred from behavioral data that the user's cognitive ability has declined, it is possible to recommend treatment with the light stimulus and sound stimulus with the gamma frequency. To a user whose disruption of sleep rhythm is inferred, it is possible to recommend treatment with early morning blue light.

In addition, a history of light to which the user is exposed in one day is grasped from the user analysis device 40 including the various types of image sensors mentioned above, and light having a wavelength that the user lacks can be estimated. For example, for a user who seldom goes out and is exposed to little red light, treatment with red light in a bathroom can be recommended.

Inspection data from a medical institution may be input into the treatment management application and used as a material for determining appropriate treatment. For example, a person whose result of a genetic test shows that the person has ε4/ε4 allele of a cholesterol metabolism factor (ApoE) has about four times a risk of dementia compared with a person having ε3/ε3 (Neutral genotype). Therefore, it is possible to recommend that treatment with the light stimulus and sound stimulus with the gamma frequency be performed for a longer time than other users.

### (7-8) Configuration of Device Supporting Plurality of Types of Treatment

As a device used in the treatment, a liquid crystal display that is provided with the following LED light sources of three colors in its backlight is conceivable. It is desirable that the LEDs are densely disposed over the entire panel so as to ensure a necessary luminance.
Blue: at or around 480 nm (usage: a therapy for dementia, adjustment of a circadian rhythm, etc.)
Green: at or around 525 nm (usage: a therapy for dementia, pain control, etc.)
Red: at or around 670 nm (usage: a therapy for dementia, PBMT, etc.)

With the configuration described above, light stimuli that support various types of treatment can be generated, and thus it is possible to provide content with an appropriate wavelength or frequency in accordance with the user's conditions.

In addition, a plurality of types of treatment can be performed simultaneously. For example, by providing the light stimulus with the gamma frequency using visual content predominantly featuring blue color (a visual content of a blue sky, etc.) at a time of waking up, an effect of preventing or providing a therapy for both dementia and SAD can be obtained.

Alternatively, by providing the light stimulus with the gamma frequency using visual content predominantly featuring red color (a visual content of a sunset glow, etc.), it is possible to treat dementia, retinopathy, dermatosis, and the like simultaneously.

The visual output device may be installed in a specific room or facility and may be configured to be portable in a form of a tablet device and placed at a required place for use as appropriate.

### (7-8) Use of System 1 as Game

In the system 1, a visual content from the display or the projector used as the visual output device, the fine particles discharged from the fine particle discharge device 30, and the like can be used as content in a game.

For example, in a case where a therapy with the light stimulus and sound stimulus with a frequency in the gamma band (25 to 100 Hz) is performed, the process of inserting OFF mentioned above is performed on a visual content and sound of the game to generate a visual content and sound that repeats ON/OFF at certain intervals.

The visual content flickers, and thus visual content-based content such as a shooter game, a racing game, or an action game is desirable, rather than content that includes a scene in which the user reads text, such as a role playing game or a quiz game.

The present disclosure can be used as a visual content of content in which a game and fitness are integrated together. For example, there is known content in which a player in a game moves forward as a user pedals a bicycle.

By reforming these types of content for performing a therapy for dementia, it is possible to take dementia measures by exercise and a therapy with the light/sound stimuli in parallel.

By converting a specific wavelength in a visual content of a game, such as blue light, red light, or green light, into a wavelength of a color suitable for the treatment, it is possible to perform the therapy while the user is playing the game.

Alternatively, a device that generates an electric stimulus or an ultrasonic stimulus to skin can be built in a game console.

### (7-9) Use of System 1 in Room for Yoga, Meditation, Or Religious Ceremony

The system 1 may be used in a room where the user practices yoga, meditation, mindfulness, or the like.

For example, it is demonstrated that watching an image of a natural scene reduces a labor needed to practice mindfulness. Thus, a visual content output from the light stimulus device 10 is desirably a visual content that imitates a natural scene.

When practicing yoga or meditation, it is important for the user to be relaxed, and a relaxed state can be grasped with a wearable device that records a heart rate or the like, such as a smartwatch. Data from these wearable devices may be obtained as therapy record data.

The system 1 can be used in a room where a religious ceremony such as prayers is held. By displaying an image of a statue of Buddha, religious painting, temple, shrine, church, mountain in which a deity is enshrined, or the like on the liquid crystal display and generating fog in front of the image, it is possible to stage sublime or sacredness.

As an existing product, for example, a statue of Buddha with LEDs is known. It is also possible to use, as a treatment device, a device in which the treatment light source is disposed in a structural object of religious significance and the fine particles such as fog are added to the structural object.

### Second Embodiment

### (8) Application Example of System 1

Next, an application example of the system 1 will be described as a second embodiment.

The same components as those in the first embodiment will be denoted by the same reference characters, and the explanation thereof will be omitted.

First, a combination of the system 1 and an inspection system for dementia will be described.

As mentioned above, the system 1 reduces a burden of continuing the treatment on the user. Additionally, grasping effects of the treatment from measurement values or a result of inspection enables the user to appreciate a concrete benefit brought by the treatment, enhancing incentive to continue using the system 1. In addition, it is possible to grasp a highly effective treatment protocol from the result of inspection.

For example, it is possible to link an execution log of stimulus treatment and a reward together. This can solve such a problem that the light treatment cannot be continued, which is not solved by a conventional system, by linking execution of the light treatment and a reward and giving a monetary incentive. Thus, it is possible to improve refractory diseases including dementia.

In addition, in cooperation with an insurance service or a financial service, the continuance of the treatment is directly linked to a monetary benefit such as financing or points that can be used in shopping or the like, thus enhancing the user's incentive to continue the treatment.

The effects described above increase the number of persons who receive the treatment, increase needs for the content included in the treatment protocol, and expand a market of the treatment content. This helps makers of the content make investments in producing content, and a quality of the content also improves. The improvement of the quality of the content produces such a virtuous cycle that the user's incentive to receive the treatment is further enhanced, and thus the user becomes healthier.

Since the content used in the system 1 is content for improving the user's health conditions, creators of the content can feel such a sense of fulfillment that production of the content leads to a social contribution.

In addition, in a case where the treatment content is evaluated based on the inspection data, by evaluating the treatment content based on the result of inspection, “content proven to be effective in the improvement by objective indices” can be selected and provided to the user. In addition, feelings of trust and security of the user in the treatment are enhanced. An inspection system using such a system 1 will be described in detail.

### (8-1) Business Model Using Inspection System Using System 1

In this inspection system, for example, the system 1 can be used together with a business model that provides content that is tailored to a preference of the user.

Here, sales channels in the business model in a case where a specific company provides, as a platform, the various devices that give the light stimulus and the sound stimulus, the management application, and an inspection device (a wearable device, etc.) for evaluating the effects brought by the treatment will be described.

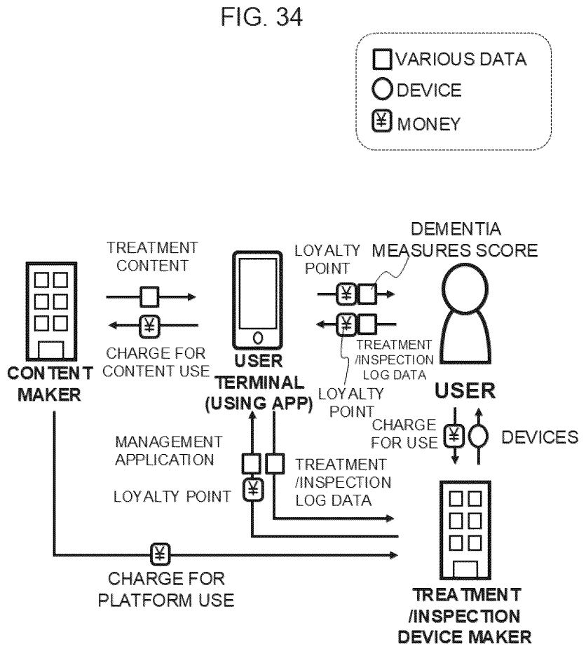

FIG. 34 is a diagram illustrating the business model using the inspection system using the system 1. This diagram illustrates flows of “various types of data,” “articles (devices),” and “money” as flows of commercial materials in the business model.

As illustrated in FIG. 34, the following players are involved in this business model.
User: a person who uses the system 1 to perform necessary treatment (primarily intended for a common consumer)
Treatment/inspection device maker: an entity that produces the system 1 and provides the system 1 to the user, and proposes an appropriate process using the system 1 to the user (primarily intended for a company that produces medical equipment)
Content maker: an entity that produces and provides video data to be the treatment content in the system 1 (primarily intended for a company such as a video production company, or an individual)

In this business, a device maker provides the user with the devices constituting the system 1 together with the management application for the treatment. At this time, the device maker may individually provide the user with a device for the inspection.

The user pays the device maker for a charge for use as compensation for the provision of the devices. At this time, the user may purchase the devices or may rent the devices and pay a charge for use based on a usage of the devices through subscription.

The device maker creates, on the management application, a sales platform on which the content maker can sell visual contents and sound content to be used in the treatment, and collects a charge for platform use from the content maker.

The content maker provides treatment content produced by itself to the sales platform and collects a charge for use of content in accordance with a usage made by the user.

The user operates the user terminal 5 to select, from among pieces of treatment content registered in the management application, a piece of treatment content intended by the user in registration of a treatment command, thus using the system 1.

At this time, the user obtains points in accordance with a frequency of use.

The user may also perform inspection using the system 1, which will be described later. A result of this inspection is recorded as a dementia measures score.

The management application that runs on the user terminal 5 obtains a log of the treatment by the system 1. In a case where the user uses the system 1 to perform inspection, the management application obtains a result of the inspection.

Here, loyalty points acquired by the user may be used as compensation for performing the inspection. That is, the system 1 may be configured as a system that allows the user to perform the inspection when the user continuously receives the treatment by the system 1. This can make the inspection an incentive to continue using the system 1.

The device maker obtains a usage log of the content selected by the user from data in the management application. By analyzing the usage log of the content, it is possible to build a system that recommends content suitable for the user. The user can efficiently obtain information on content that is tailored to a preference of the user, thus bringing benefits to both the maker and the user.

The device maker may also provide a device for inspecting dementia at the same time for monitoring the effects of the treatment. For example, the user analysis device 40 may be used in the system 1 to build the following systems of evaluating a cognitive function of the user to perform inspection of dementia.
A system of measuring a motor function of the user with motion sensors (an acceleration sensor or a gyro sensor, etc.) that are built in a wearable device and a smartphone
A system of evaluating a circadian rhythm with a sleep monitoring device
A system of evaluating a cognitive function of the user by analyzing the user's speed of typing on a smartphone

The inspection of the cognitive function of the user by the system 1 described above is poor in accuracy compared with inspection in a medical institution but is suitable for detecting particularly a change seen in an initial stage of dementia because the inspection by the system 1 can be used routinely. A monitoring system using the devices constituting the system 1 enables the user to continue the treatment while confirming that the user's dementia does not progress or improves, from concrete data.

In addition, in a case where a tendency of cognitive function decline is observed from a result of measurement by the inspection system, it is also possible to urge the user to receive inspection in a medical institution, thus preventing a delay in starting a therapy.

### (8-2) Business Model Using Insurance/Financial System Using System 1

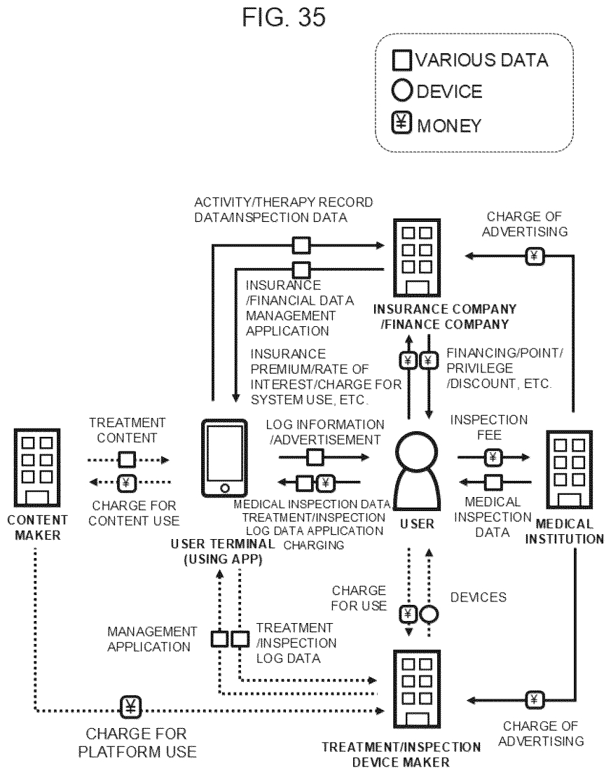

Next, a usage method and a business model in a case where the system 1 is applied to an insurance business or a financial business will be described. FIG. 35 is a diagram illustrating the business model using an insurance/financial system using the system 1. In this diagram, the same flows of commercial materials as in FIG. 34 are illustrated with broken lines, and descriptions thereof will be omitted. Additional players involved in this business model are as follows.
Medical institution: an institution that provides medical treatment for a specific disease such as dementia (intended for a hospital)
Insurance company, finance company: an entity that evaluates a physical risk of the user based on information obtained from the system 1 and provides an insurance service or financing

In this business model, the user registers himself or herself in a specific insurance system or financial system and causes the treatment management application to cooperate with the specific insurance system or financial system. A log of the treatment received by the user or a result of the inspection obtained from the inspection device is sent to an application of the insurance service or the financial service and is reflected in evaluation of an insurance premium, financing, or the like.

A history of visits to inspection in a medical institution or the like can be subjected to the evaluation. For example, inspection such as brain fMRI image diagnosis, amyloid PET imaging, blood test (amyloid peptide content, etc.), genetic test (APoE genotype, etc.), cerebrospinal fluid test (CSF tau, amyloid β, ferritin, iron-related proteins), electroencephalography (EEG), cognitive ability test, or retinal image diagnosis can be used. A result of inspection is sent by the user to the application of the insurance service or the financial service.

The data or the diagnosis result described above is evaluated from, for example, the following viewpoints, and a privilege based on an evaluation score such as points, a discount, or preferential treatment in financing is provided to the user.
Performing the treatment for a certain period of time or longer at a certain frequency or higher (a total of durations of the treatment that is received in various places such as a bathroom, toilet, or bedroom)
Undergoing inspection periodically
A result of inspection meeting certain criteria or improving

For example, in a case where the user executes the treatment with a stimulus with the gamma frequency every day and the user's cognitive performance and an accumulation level of amyloid β falls within their normal ranges, it can be determined that the user is highly likely to be able to maintain cognitive ability for a long term. Therefore, the user can be a target of a reward such as reducing an insurance premium or reducing a rate of interest in financing.

By displaying an advertisement of a medical institution that inspects dementia on a screen of the management application or the application for managing the insurance/financial system, it is possible to provide information on inspection that is tailored to the user's needs. For an inspection company, this leads to an increase in users, and an increase in profits can be expected. Therefore, it is possible to build a system that provides benefits to both.

In addition, as inspection data is accumulated, content that is effective in maintaining or improving a cognitive function becomes clear. Thus, the more the user uses the system, the more effectively the user can prevent dementia.

The system described above may be applied to usage not only for the gamma stimulus therapy but also for prevention or improvement of other types of disorders in health conditions that are expected to be improved by the use of the system 1, such as seasonal affective disorder, winter depression, jet lag, retinopathy, pain control, and wellness improvement.

(8-3) Case Where Insurance Company Provides Platform of Treatment of/Inspection for Dementia

As a more specific example, an ICT system in which the system 1 and an insurance service are integrated together will be described.

In a typical insurance service, the user (or a company to which the user belongs) pays an insurer (an insurance company, etc.) an insurance premium, and the insurer bears part of the user's consultation fee when the user visits a medical institution and part of a compensation for temporary disability due to a disease. The insurer may pay a medical institution a medical fee.

Thus, when the user's health conditions including a cognitive function improve through the use of the system 1, the insurer can reduce the burden to increase a profit and thus can reduce the insurance premium. An example of a specific insurance service will be described below.

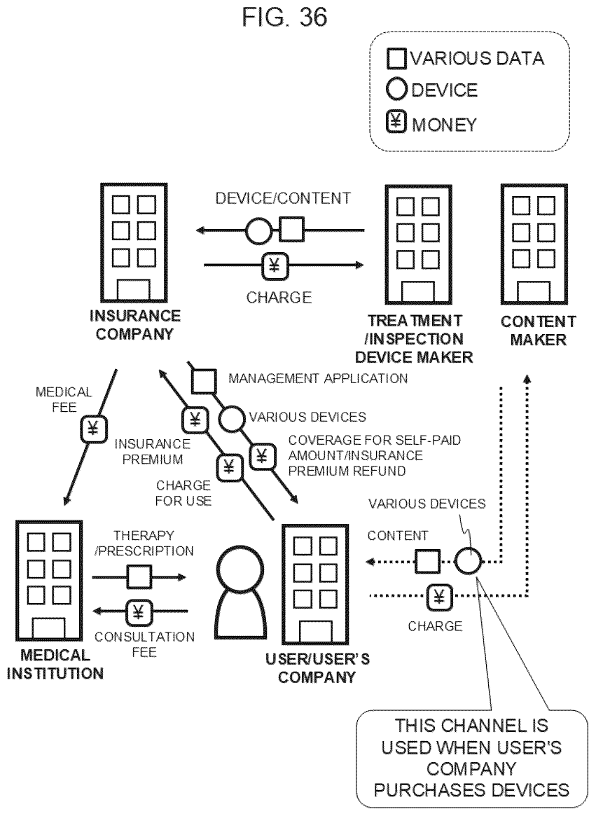

FIG. 36 is a diagram illustrating a business model in which an insurance company provides a platform of stimulus treatment of/inspection for dementia. In this diagram, the same parts as in FIG. 34 and FIG. 35 mentioned above are illustrated with broken lines, and descriptions thereof will be omitted.

As illustrated in FIG. 36, the user pays the insurer a charge for use of the service, and the insurer supplies the user with devices (a stimulus treatment device, an inspection device) purchased from a device maker and an application for management (hereinafter, called insurance application). The device may be purchased by the user or the company to which the user belongs.

The user uses the insurance application on a smartphone or a terminal and uses the stimulus treatment device and the inspection device according to a guide on the application. The insurance application is desirably a web application that can be accessed from a web browser so as to allow various terminals to access the insurance application.

Based on a log of the treatment and a result of inspection on the user, the insurance application evaluates a dementia measures level of the user and gives the user a reward such as a discount on the insurance premium in accordance with a result of the evaluation. In addition, based on evaluation of content, the insurance application recommends details of treatment (treatment content, etc.) that the user should receive in the future.

A user who takes dementia measures appropriately is given many rewards, which give the user an incentive to continue the treatment. In addition, since the details of treatment to be executed in the future are recommended, it is possible to take more effective measures, and thus the more the user uses the system 1, the more the user's health conditions improve.

Since the effects of the treatment can be objectively evaluated from the log of the result of the inspection, it is possible to select highly effective content, and thus a content maker that creates high quality content can sell more content.

An insurance system 2 that performs such a process will be described below.

### (8-3-1) Configuration of Insurance Management Server 90

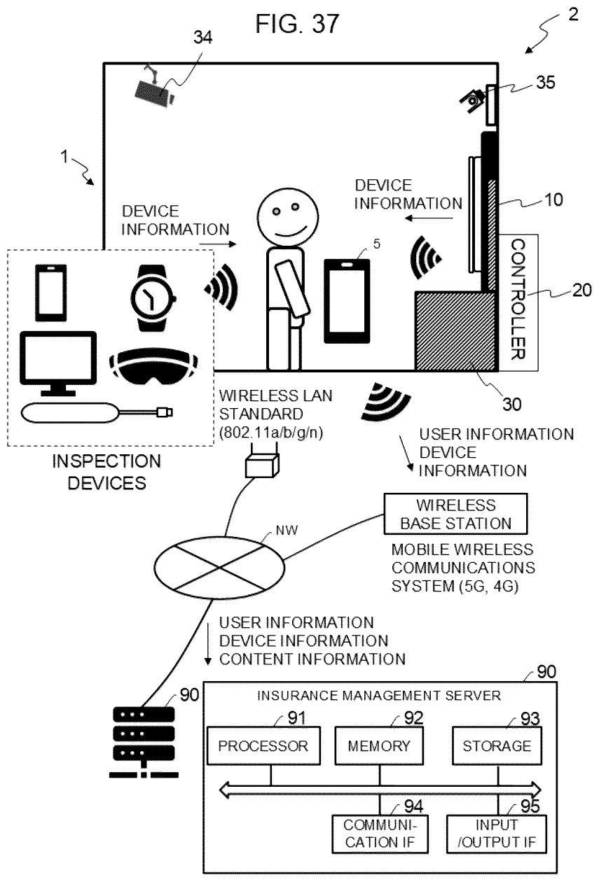

FIG. 37 is a diagram illustrating a communication state of data at a time of starting use of the insurance system 2 that uses the system 1.

As illustrated in FIG. 37, the insurance system 2 includes an insurance management server 90 that is used together with the system 1 (hereinafter, simply referred to as a management server 90).

The management server 90 includes a processor 91, a memory 92, a storage 93, a communication IF 94, and an input/output IF 95.

The processor 91 is a piece of hardware for executing a set of instructions written in a program stored in the memory 92. The processor 91 is constituted by an arithmetic unit, registers, peripheral circuits, and the like.

The processor executes a program pertaining to the insurance application to execute, for example, the following processes.
Obtaining a log of the treatment by the system 1
Obtaining a result of the inspection
Analyzing the log of the treatment and the result of the inspection (evaluating the user and good content)
Recommending the good content

These processes will be described later in detail.

The memory 92 is for temporarily storing, for example, programs and data to be processed by the programs or the like.

For example, the memory 92 is a volatile memory such as a dynamic random access memory (DRAM).

The programs include, for example, the following programs.
A program of operating system (OS)
A program pertaining to the insurance application
A program of a web browser that performs information processing

The storage 93 is a storage device for saving data. For example, the storage 13 is a flash memory, a hard disc drive (HDD), or a solid state drive (SSD).

The communication IF 94 is an interface through which the system 1 inputs and outputs signals to communicate with external equipment. As the communication IF 94, specifically, a module that supports a general-purpose telecommunications standard is desirably used.

The input/output IF 95 functions as an interface for an input device (e.g., a pointing device such as a mouse, a keyboard, etc.) for receiving input operations from the user and output devices (a display, the speakers 63, etc.) for presenting information to the user.

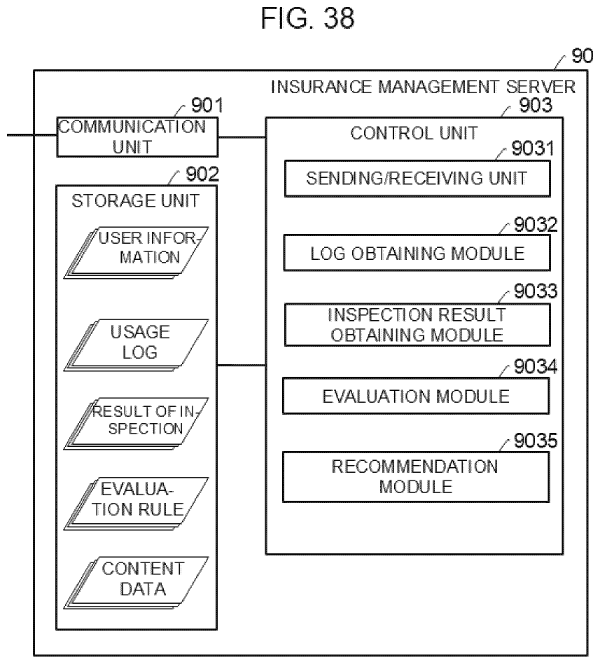

Next, a functional configuration of the management server 90 will be described. FIG. 38 is a diagram illustrating the functional configuration of the management server 90.

The management server 90 exercises functions as a communication unit 901, a storage unit 902, and a control unit 903.

The communication unit 901 performs processing in which the management server 90 communicates with the external equipment.

The storage unit 902 stores data and programs to be used by the management server 90. The storage unit 202 stores user information, a usage log, a score, a result of inspection, an evaluation rule, and treatment content.

The user information is personal information on a user that is stored for each user ID. The user information includes a name, an age, a sex, a place of residence, an occupation, and the like.

The user information may include the following types of information.
Device information on a user terminal 5 used by a user
Details of an insurance contract of a user
Target of a physical function that a user intends to achieve by using the system 1
User's favorite treatment content preferred by a user in the system 1
An evaluation score given to a user

The usage log is information that shows a history of treatment that a user has performed using the system 1. The usage log includes at least the following.
Date and time of use
A user ID of a user who uses the system 1
Details of a treatment protocol (information on treatment content, output intensity, output duration, etc.)

The result of inspection is information in which a result of inspection by an inspection device is associated with a user ID.

The result of inspection may include, for example, a result of medical examination that is input by a user and is not obtained from the inspection device.

The evaluation rule is information in which a criterion of the evaluation in the insurance system 2 is defined. As the evaluation rule, at least the following types of information are included.
A criterion of evaluation pertaining to a primary score that is given in accordance with a frequency of use by a user
A criterion of evaluation pertaining to a weighting coefficient that is given in accordance with a result of inspection
Definition pertaining to details of a reward based on an evaluation score
A criterion pertaining to evaluation of good content

Note that the evaluation score is calculated as a value that is the primary score multiplied by the weighting coefficient.

The content data is content data as a material that is to be converted into treatment content.

The content data may be stored being associated with a content score.

The control unit 203 exercises functions as a sending/receiving unit 9031, a log obtaining module 9032, an inspection result obtaining module 9033, an evaluation module 9034, and a recommendation module 9035 as the processor 21 of the management server 90 executes the insurance application.

The sending/receiving unit 2031 controls a process in which the management server 90 sends a signal to the external equipment under a communications protocol and a process in which the management server 90 receives a signal from the external equipment under the communications protocol.

The log obtaining module 9032 obtains a log of treatment by the system 1 from the application on the user terminal 5. The log obtaining module 9032 stores the obtained log in the storage unit 902. That is, the log obtaining module 9032 records a duration of the treatment and a frequency of the treatment in the storage unit 902 as a record of the light treatment.

The inspection result obtaining module 9033 obtains a result of inspection of a user from the inspection device. The inspection result obtaining module 9033 stores the obtained result of inspection in the storage unit 902. That is, the inspection result obtaining module 9033 obtains a result of inspection pertaining to a cognitive ability of the user.

The evaluation module 9034 evaluates a user and gives the user an evaluation score from a history of use of the system 1 by a user and the result of inspection, according to a process described later. The evaluation module 9034 stores a result of the evaluation in the storage unit 202. That is, the evaluation module 9034 evaluates a user based on records of the light treatment being recorded and a transition of a cognitive ability of the user that is confirmed from the records of the light treatment and the result of the inspection.

The evaluation module 9034 also evaluates treatment content and determines good content by comparing results of the inspection of a plurality of users. The evaluation module 9034 stores a result of the evaluation in the storage unit 202.

The recommendation module 9035 recommends the good content to a user based on a result of the evaluation of treatment content by the evaluation module 9034.

Next, a process by the insurance system 2 will be described.

The process by the insurance system 2 is divided into the following steps.
Step 1: Starting use of the insurance application and registering a device
Step 2: Obtaining a log of treatment and a result of inspection
Step 3: Analyzing the log of the treatment and evaluating a dementia measures level of a user
Step 4: Providing a recommendation to the user based on evaluation of content

These steps will be described below.

### (8-3-2) Step 1: Starting Use of Insurance Application And Registering Device

A user uses the user terminal 5 to make usage registration of the insurance application. The user inputs, from an input device such as a keyboard or a touch panel, user information such as a user ID (e.g., an email address), a password, an age, and a sex.

As illustrated in FIG. 37, the input user information is sent from the user terminal 5 via the network to the management server 90 that manages the insurance application. The sent information is saved in a user database in the memory via a communication module of the management server 90.

Next, the user registers, in the insurance application, a treatment device and an inspection device to be used. Address information in the device information is sent from communication modules of the devices to a communication module of the user terminal 5 in near field communication or via a wireless communication network and is saved in a memory of the user terminal 5. The saved device information is sent from the user terminal 5 to the management server 90 via the network such as the Internet and is stored in the storage unit 902.

By the process described above, the device information on the user terminal 5 associated with the user ID is saved in the storage unit 902 of the management server 90 as the user information.

### (8-3-3) Step 2: Obtaining Log of Treatment and Result of Inspection

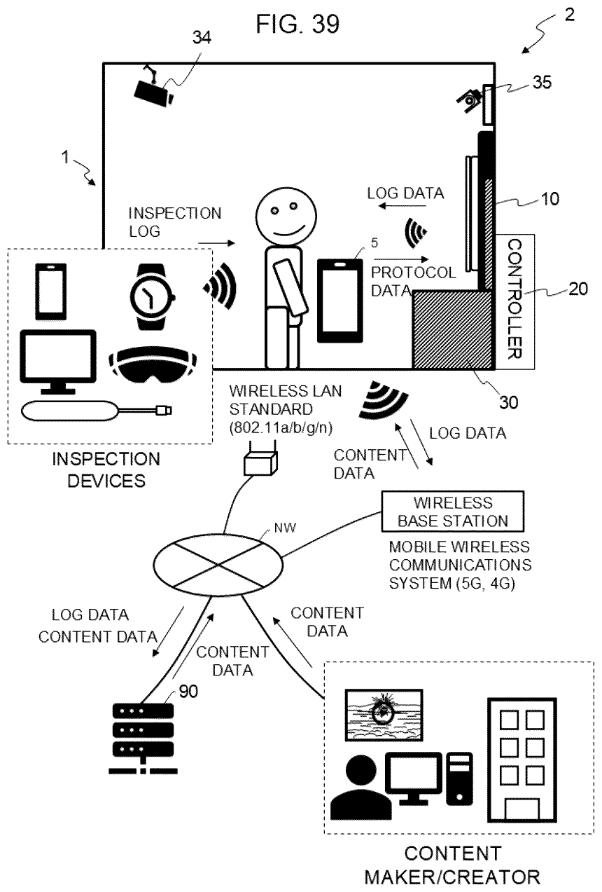

FIG. 39 is a diagram illustrating a communication state of data at a time of using the insurance system 2 that uses the system 1.

First, in the insurance system 2, the system 1 executes the treatment, and a log of the treatment is obtained.

Content data used in the treatment is generated in advance by a producer such as a content maker or a creator. The content data is sent to the communication module of the management server 90 via the network together with text data such as a title, a producer name, and a category, and stored in the storage unit 902.

Content that can be used is displayed on a graphical user interface (GUI) of the insurance application.

The user can search for content to be output in use of the system 1 with a category or a keyword by operating the insurance application that runs on the user terminal 5.

For example, the user sends a request via the network from a search menu in the insurance application. The processor of the management server 90 compares the text data in the storage unit 902 and content of the request, determines content that matches the request, sends the content via the network, and displays the content on the GUI of the insurance application. The user selects content that is tailored to a preference of the user and other settings (an amount of light, a duration of the treatment, etc.) to determine a treatment protocol.

The insurance application sends the treatment protocol selected by the user from the user terminal 5 to the controller 20 of the system 1 in wireless communication, and the user receives the treatment according to the specified protocol.

The log obtaining module 9032 of the management server 90 obtains logs of the treatment. Specifically, the logs of the treatment are sent from the devices to the user terminal 5 in wireless communication and are saved in the memory of the user terminal 5. The saved logs of the treatment are sent from the user terminal 5 to the management server 90 via the network such as the Internet. The log obtaining module 9032 stores the sent logs of the treatment in the storage unit 902 as a usage log of the user.

Next, obtaining a result of inspection from the inspection device will be described.

The user uses the inspection device to inspect a cognitive function. Here, the inspection may be performed using an inspection device that constitutes the user analysis device 40 of the system 1 or using an individual inspection device. A specific inspection device and details of the inspection include, for example, the following details.

In a case where the inspection is performed with a smartphone, a tablet computer, or a PC, the following can be cited, for example.
A speed of typing on the input device
A score of a dementia test application
Scores of subjective sleep quality, mood, and the like through a questionnaire
Evaluation of an amount of exercise based on changes in positional information on the smartphone

In a case where the inspection is performed with a wearable device, the following can be cited, for example.
Evaluation of an amount of exercise of the user
Evaluation of a phase of a circadian rhythm using biological information on the user
Evaluation of a sleep quality (evaluated based on how short a duration of activities in the nighttime is and how low a frequency of the activities in the nighttime is)

In a case where the inspection is performed with a sleep monitoring device, the following can be cited, for example.
Evaluation of a phase of a circadian rhythm using biological information on the user
Evaluation of a sleep quality (evaluated based on how short a duration of activities in the nighttime is and how low a frequency of the activities in the nighttime is)

As inspection data from an inspection institute (a manual input on the insurance application by the user), the following can be cited, for example.
fMRI image diagnosis
Amyloid PET imaging
A blood test (amyloid peptide content, etc.)
A genetic test (APoE genotype, etc.)
A cerebrospinal fluid test (CSF tau, amyloid β, ferritin, iron-related protein)
Electroencephalography (EEG)
A result of a cognitive ability test
Retinal image diagnosis Note that inspection other than these may be performed.

The inspection data obtained from the types of inspection described above is sent from the inspection devices to the user terminal 5 and is saved in the memory of the user terminal 5. The saved logs of the treatment are sent from the user terminal 5 to the management server 90 via the network. The inspection result obtaining module 9033 obtains the sent result of the inspection and stores the result of the inspection in the storage unit 902.

### (8-3-4) Step 3: Analyzing Log of Treatment And Evaluating Dementia Measures Level of User

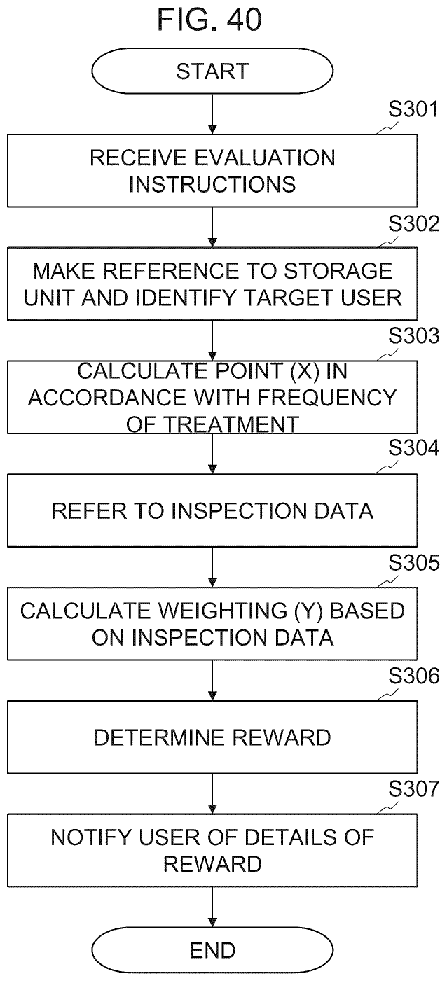

FIG. 40 is a chart illustrating a process performed by the evaluation module 9034 of the management server 90.

As illustrated in FIG. 40, the evaluation module 9034 of the management server 90 receives evaluation instructions (step S301).

Specifically, the evaluation module 9034 receives the evaluation instructions that are input from the insurance application at preset time intervals (e.g., on a daily basis, etc.).

Next, the evaluation module 9034 makes reference to the user information to identify a target user (step S302).

Specifically, the evaluation module 9034 refers to user information for a user to be evaluated, identifies a user ID from the device information, and identifies a usage log and a result of inspection that are associated with the user ID.

Next, the evaluation module 9034 gives a primary score that is given in accordance with a frequency of the treatment (step S303).

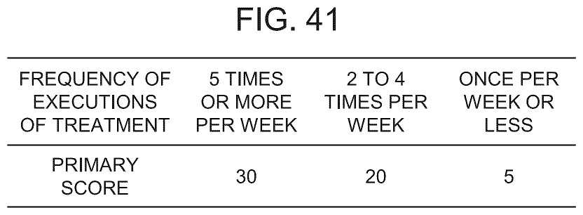

Specifically, the evaluation module 9034 refers to a log of the treatment and a criterion of evaluation that are stored in the storage unit 902 and calculates the primary score based on a frequency and a duration of the treatment. FIG. 41 is a table illustrating an example of a criterion of evaluation pertaining to a primary score that is given in accordance with a frequency of use by a user.

As illustrated in FIG. 41, primary scores based on frequencies of executions of the treatment are set as an evaluation rule.

In the illustrated example, for example, the evaluation rule is defined such that 30 points are given to a user who receives the treatment 5 times or more a week, 20 points are given to a user who receives the treatment 2 to 4 times a week, and 5 points are given to a user who receives the treatment once a week or less. Note that the criterion of evaluation for a primary score based on a frequency of executions of the treatment can be changed freely.

After step S303, the evaluation module 9034 refers to inspection data (step S304).

Specifically, using the user ID, the evaluation module 9034 identifies the result of inspection stored in the storage unit 902 about the user being an evaluation target.

After step S304, the evaluation module 9034 calculates a weighting based on the inspection data (step S305).

Specifically, the evaluation module 9034 evaluates the result of inspection based on a target range of an inspection score. That is, the evaluation module 9034 evaluates a transition of a cognitive ability of the user by comparing a value indicated by the result of inspection with a reference value that is preset. Here, cases where the transition of the cognitive ability is evaluated include the following patterns.
A case where the cognitive ability improves through the use of the system 1
A case where the cognitive ability is maintained through the use of the system 1
A case where it is recognized that a decline in the cognitive ability has been slowed more than assumed through the use of the system 1

The target range of the inspection score can be set based on an average score of healthy individuals (individuals who have mild cognitive impairment or have not developed dementia) and average score data on individuals who have cognitive impairment.

For example, regarding a speed of typing, the following criteria are known.
Healthy individuals: an average of 115 (keystrokes/min, ±11 in 95% confidence interval)
Individuals who have symptoms of cognitive impairment: an average of 87 (keystrokes/min, ±10 in 95% confidence interval)

Based on these, a range of 115±11 can be set as the target range.

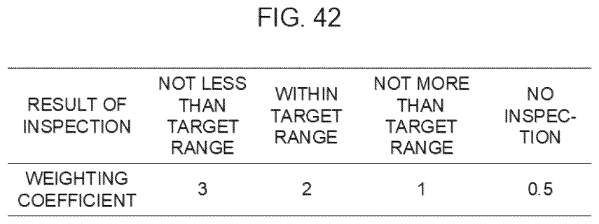

Data on the target range is created in advance by an insurance company or the like and is stored in the storage unit 902 as a criterion of evaluation. FIG. 42 is a table illustrating an example of a criterion of evaluation pertaining to a weighting coefficient that is given in accordance with a result of inspection.

In the example in FIG. 42, a weighting coefficient (a Y value) is set as follows.
3 for a user whose inspection score is not less than the target range
2 for a user whose inspection score is within the target range
1 for a user whose inspection score is not more than the target range
0.5 for a case where the inspection is not performed

In this example, by giving a higher value to a user who receives the inspection than a user who does not receive the inspection, it is possible to increase the evaluation score by receiving the inspection even when the primary score based on a frequency of uses is low, and it is possible to give a user an incentive to receive the inspection.

Then, the evaluation module 9034 compares the result of the inspection of the user to be evaluated with target range data that is stored as a criterion of evaluation. From the comparison between the result of the inspection and the target range, the evaluation module 9034 calculates a weighting (the Y value). Note that the target range may be set for each user. In this case, an evaluation rule stored in the storage unit 902 is associated with a user ID.

After step S306, the evaluation module 9034 determines a reward for the user (step S306).

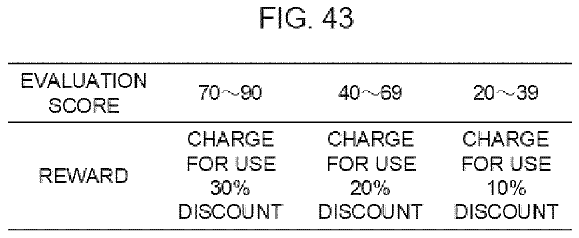

Specifically, the evaluation module 9034 calculates, as a score indicating the dementia measures level, a value that is a multiplication of an X value and the Y value and determines a reward to be provided to the user based on the calculated value. FIG. 43 is a table illustrating an example of a definition pertaining to details of a reward based on an evaluation score.

In the example illustrated in FIG. 43, a discount on an insurance premium is set as the details of a reward. As illustrated, a discount rate increases with an increase in the evaluation score of the user, thus bringing more benefits. Details of the determined reward are stored in the storage unit 902 in association with the user information.

After step S306, the evaluation module 9034 notifies the user of the details of the reward (step S307).

Specifically, the evaluation module 9034 sends information about the details of the reward to the user terminal 5 via the network. The user terminal 5 displays the information about the details of the reward on the GUI of the running insurance application, thus presenting, to the user, the reward to be given. The process of the evaluation module 9034 is thus finished.

Next, a process of determining a reward in a service for a corporation will be described.

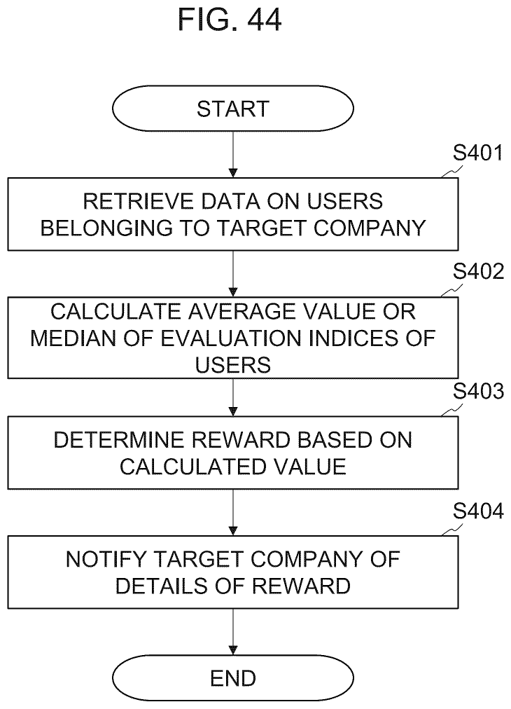

FIG. 44 is a chart illustrating a process at a time when a reward is provided to a corporation.

As illustrated in FIG. 44, when the present system is provided as a service for a corporation, an insurance premium is paid by a company to which a user belongs, rather than the user. In this case, for example, a reward to the corporation is determined based on an average value or a median of values of evaluation index of all users who belong to the corporation.

The above process is executed every certain period of time specified by a service provider, such as a week, month, or year, and data is accumulated.

Specifically, the evaluation module 9034 first retrieves data on users who belong to a target company (step S401).

Specifically, the evaluation module 9034 uses a company ID of the target company to extract the users from a user DB and identifies a log of the treatment of each of the users and inspection result data on the user.

After step S401, the evaluation module 9034 calculates the average value or the median of evaluation indices of the users (step S402).

After step S402, the evaluation module 9034 determines a reward based on the calculated value (step S403).

After step S403, the evaluation module 9034 notifies the company of details of the reward (step S404).

Specifically, the evaluation module 9034 sends information about the details of the reward to a computer system of the corporation being the evaluation target.

The process of determining the reward in the service for a corporation is thus finished.

### (8-3-5) Step 4: Providing Recommendation to User Based on Evaluation of Content

Furthermore, the insurance system 2 is capable of providing a user a recommendation based on evaluation of content. That is, the insurance system 2 provides evaluation of treatment content and a recommendation based on comparison and analysis for each user.

The recommendation module 9035 performs the comparison and analysis for each user based on the accumulated data. First, the recommendation module 9035 reads data in a user database and extracts “a user whose value continuously increases” or “a user who constantly gets a high value” from a transition of the Y value (the weighting based on a result of the inspection) for every certain period of time (e.g., on a monthly basis). That is, the recommendation module 9035 extracts a user group that shows a suitable tendency in the transition of the cognitive ability in results of inspection and identifies visual content that is used with high frequency in the extracted user group. Here, the user group that shows a suitable tendency in the transition of the cognitive ability includes the following.
A user group exhibiting improved cognitive ability through the use of the system 1
A user group exhibiting maintained cognitive ability through the use of the system 1
A user group for which it is recognized that a decline in the cognitive ability has been slowed more than assumed through the use of the system 1

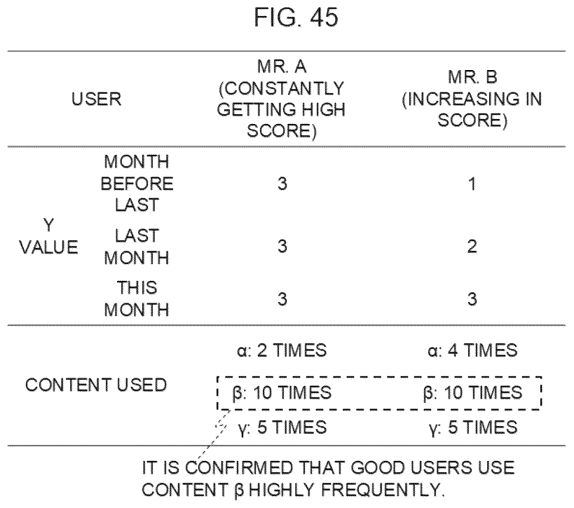

FIG. 45 is a diagram illustrating an example of evaluation of good content.

As illustrated in FIG. 45, a score of content can be calculated as follows based on a cumulative number of uses.

$$Content α : 2 + 4 = 6$$

$$Content β : 10 + 10 = 20$$

$$Content γ : 5 + 5 = 10$$

Then, the evaluation module 9034 calculates pieces of treatment content used by the extracted users (good users) for a predetermined period (e.g., three months) and the numbers of uses of the pieces of treatment content and identifies content that tends to be used with high frequency by the good users.

As a specific calculation method, for example, the numbers of uses by the good users are calculated as evaluation scores for the content, and by totalizing values that are calculated for all good users, an evaluation score for the content is determined.

Here, content that is repeatedly used by a good user is highly likely to meet both an “improvement effect in cognitive ability,” and “favorability” and is thus considered to be content with high quality. The evaluation of content is repeated, and a score of content is constantly updated.

Next, the recommendation module 9035 of the management server 90 sends a command to retrieve content to be displayed to the management server 90 via the network when the user opens a screen for setting a treatment protocol or searches for content. The recommendation module 9035 refers to scores of pieces of content and sends the pieces of content in descending order of the score.

The sent pieces of content are displayed on the insurance application as “recommended content” or “top ranked content.” The above process makes it easy for a user to select good content, and the user can receive treatment that is more favorable and more effective.

### (9) Other Modifications

The configuration of the system 1 can be modified as appropriate.

For example, in the embodiments described above, the configuration in which the system 1 includes the light stimulus device 10 including the light source 11 and the visual display unit 12 and includes the fine particle discharge device 30 is shown. However, this is not limitative. The system 1 may include the light source 11 and the fine particle discharge device 30, without the visual display unit 12. This is because the light stimulus output from the light source 11 can be scattered by the fine particles and can be input into the user as a light stimulus that has a moderately lessened intensity.

That is, in the system 1, the visual display unit 12 and the fine particle discharge device 30 have a common function in that the visual display unit 12 and the fine particle discharge device 30 both soften the treatment light applied from the light source 11. Therefore, only one of the visual display unit 12 and the fine particle discharge device 30 may be included, and the visual display unit 12 and the fine particle discharge device 30 can be replaced with each other.

The system 1 need not include at least any one of the user analysis device 40, the voice recognition device 50, the sound stimulus device 60, and the cutaneous stimulus device 70. In this case, although an advantage in convenience brought by each of the devices is reduced, it is possible to perform the treatment with the light stimulus device 10 and the fine particle discharge device 30.

The blinking of the light source 11 in the system 1 is not necessarily the alternation of turning on (ON) and turning off (OFF). That is, the blinking refers to alternation of increase and decrease in illuminance of light having a wavelength in a visible light region and includes a mode in which light is constantly turned on (ON) and changes in illuminance at a predetermined frequency. This is because changes in illuminance can input a visual stimulus into the user. That is, it suffices that the light source 11 alternates a high illuminance state and a low illuminance state, and thus it is not necessary to completely turn off the light source 11 at a timing of the low illuminance state.

Note that a frequency of a waveform of an output current of a common LED lighting instrument is required to be 100 Hz or more so as to prevent a problem such as a flicker of light from arising. In contrast, the light source 11 of the system 1 uses an effect that is obtained by a visual stimulus of light that blinks at a low frequency. Thus, it is desirable that a frequency of the blinking is less than 100 Hz (a period of the blinking is longer than 10 msec).

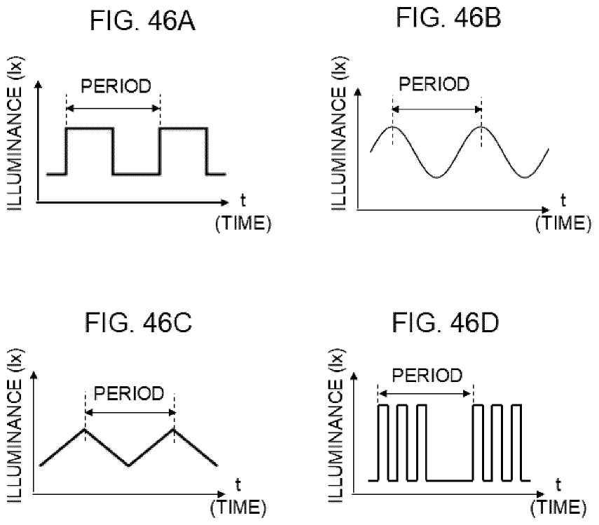

FIG. 46A, FIG. 46B, FIG. 46C, and FIG. 46D are diagrams illustrating examples of periodic changes in illuminance in another modification of the light source 11.

The light source 11 of the system 1 may change in illuminance along, for example, the following waveforms.
FIG. 46A: an illuminance change along a square wave
FIG. 46B: an illuminance change along a sine wave
FIG. 46C: an illuminance change along a triangle wave
FIG. 46D: an illuminance change that is a periodic rise and fall in average illuminance caused by changes in frequency of pulses

Then, when a waveform of alternation of increase and decrease in illuminance with a certain period T is subjected to a Fourier transform to create a frequency spectrum into which the alteration is decomposed into a sine wave and a cosine wave having a certain frequency, a highest peak is obtained at a frequency f=1/T. For example, in a case where the period is 25 msec, the highest peak is obtained at a frequency of 40 Hz. That is, as mentioned above, the blinking frequency in the system 1 refers to a frequency at which a highest peak is obtained in a frequency spectrum obtained by performing the Fourier transform on a waveform of changes in illuminance with time.

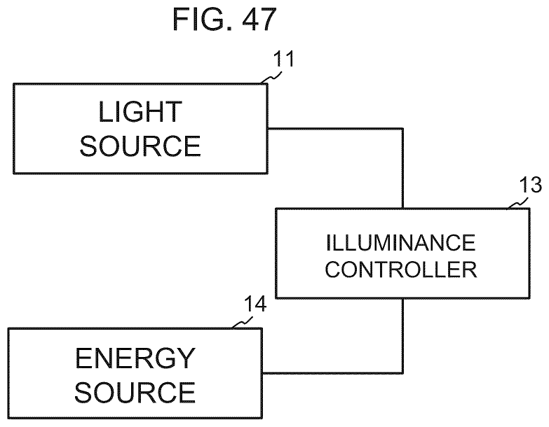

FIG. 47 is a diagram illustrating the other modification of the light source 11.

As illustrated in FIG. 47, the light source 11 that changes in illuminance is used together with an illuminance controller 13 that controls the illuminance. The illuminance controller 13 can change an illuminance of the light source 11 in accordance with a mode of the illuminance change mentioned above by controlling a voltage or a current to be input into the light source 11 from an energy source 14 such as a power source.

In the system 1, it is also effective to impart a certain periodicity to output intensities of the sound stimulus and the cutaneous stimulus. That is, the output intensities of the sound stimulus output by the sound stimulus device 60 and the cutaneous stimulus output by the cutaneous stimulus device 70 are also controlled as with the light stimulus. Specifically, in a frequency spectrum obtained by performing the Fourier transform on a waveform of changes in intensity of each of the stimuli, it is desirable that a frequency at which a highest peak is obtained (a frequency corresponding to the blinking frequency) is in a predetermined frequency band (lower than 100 Hz, more preferably, the gamma band).

The embodiments of the present invention are described above in detail. Note that the scope of the present invention is not limited to the above embodiments. In addition, the above embodiments may be subjected to various improvements or modifications without departing from the gist of the present invention. Furthermore, the above embodiments and modifications may be combined.

### (10) Supplements

The matters described in the embodiments described above will be supplemented below.

### (Supplement 1)

A treatment light control system 1 including:
a light source 11 that applies blinking light;
a controller 20 that controls a blinking frequency of the light source 11 such that the blinking frequency is included in a predetermined frequency band; and
fine particle discharge means 30 for discharging fine particles into a spatial region to which the light source 11 applies the light.

### (Supplement 2)

A treatment light control system 1 including:
a light source 11 that applies blinking light toward mainly front where a user is positioned;
a controller 20 that controls a blinking frequency of the light source 11 such that the blinking frequency is included in a predetermined frequency band; and
a visual display unit 12 that is disposed in front of the light source 11 and displays a predetermined visual content on a display surface by transmitting light applied from the light source 11.

### (Supplement 3)

The treatment light control system 1 according to supplement 1 or 2, wherein
the light source 11 occupies a certain region orthogonally mainly to a direction of applying the light, and
a blinking frequency of at least a certain region of the light source 11 is less than 100 Hz.

### (Supplement 4)

The treatment light control system 1 according to supplement 1, wherein the light source 11 is a part of a visual output device.

### (Supplement 5)

The treatment light control system 1 according to supplement 1 or 2, further including user analysis means 40 for detecting any one of a position, an attitude, a line of sight, and a hypnagogic state of a user.

### (Supplement 6)

The treatment light control system 1 according to supplement 1 or 2, further including sound stimulus means 60 for outputting a sound stimulus to be input into a user via a sense of hearing, a frequency of alternation of increase and decrease in output intensity of the sound stimulus being included in a predetermined frequency band.

### (Supplement 7)

The treatment light control system 1 according to supplement 1 or 2, further including cutaneous stimulus means 70 for outputting an electric or ultrasonic stimulus to be input into a user via a sense of touch, a frequency of alternation of increase and decrease in output intensity of the electric or ultrasonic stimulus being included in a predetermined frequency band.

### (Supplement 8)

The treatment light control system 1 according to supplement 1 or 2, wherein the treatment light control system 1 is provided and used in a bathroom.

### (Supplement 9)

A method executed by a treatment light control system 1 including a computer, wherein
a processor of the computer executes:
a step of, by a light source 11, applying blinking light;
a step of, by a controller 20, controlling a blinking frequency of the light source 11 such that the blinking frequency is included in a predetermined frequency band; and
a step of, by fine particle discharge means 30, discharging fine particles into a spatial region to which the light source 11 applies the light.

### (Supplement 10)

A program executed by a treatment light control system 1 including a computer,
the program causing a processor of the computer to execute:
a step of, by a light source 11, applying blinking light;
a step of, by a controller 20, controlling a blinking frequency of the light source 11 such that the blinking frequency is included in a predetermined frequency band; and
a step of, by fine particle discharge means 30, discharging fine particles into a spatial region to which the light source 11 applies the light.

### (Supplement 11)

A method executed by a treatment light control system 1 including a computer, wherein
a processor of the computer executes:
a step of, by a light source 11, applying blinking light toward mainly front where a user is positioned;
a step of, by a controller 20, controlling a blinking frequency of the light source 11 such that the blinking frequency is included in a predetermined frequency band; and
a step of, by a visual display unit 12, displaying a predetermined visual content on a display surface by transmitting light applied from the light source 11, the visual display unit 12 being disposed in front of the light source 11.

### (Supplement 12)

A program executed by a treatment light control system 1 including a computer,
the program causing a processor of the computer to execute:
a step of, by a light source 11, applying blinking light toward mainly front where a user is positioned;
a step of, by a controller 20, controlling a blinking frequency of the light source 11 such that the blinking frequency is included in a predetermined frequency band; and
a step of, by a visual display unit 12, displaying a predetermined visual content on a display surface by transmitting light applied from the light source 11, the visual display unit 12 being disposed in front of the light source 11.

### (Supplement 13)

The method according to supplement 9 or 11, wherein the processor further executes a step of detecting any one of a position, an attitude, a line of sight, and a hypnagogic state of a user.

### (Supplement 14)

The method according to supplement 9 or 11, wherein the processor further executes a step of turning off an illuminating device 80.

### (Supplement 15)

The method according to supplement 9 or 11, wherein the processor further executes a step of, by an optical sensor 81, measuring an amount of light in a space where a user receives treatment.

### (Supplement 16)

A method executed by a treatment light control system 1 including a computer, wherein
a processor of the computer executes:
a step of converting visual content into a visual signal that blinks at a predetermined interval;
a step of outputting, in a form of a visual content, the blinking visual signal from a visual output device; and
a step of discharging fine particles in front of the visual output device.

### (Supplement 17)

The method according to supplement 16, wherein
the processor further executes:
a step of converting sound content into a sound signal that is modulated to a predetermined interval; and
a step of outputting the sound signal.

### (Supplement 18)

A method executed by a treatment light control system 1 including a computer, wherein
a processor of the computer executes:
a step of recording a duration of treatment and a frequency of the treatment as a record of light treatment;
a step of evaluating a user based on a record of the light treatment being recorded; and
a step of calculating a reward based on a result of evaluating the user.

### (Supplement 19)

The method according to supplement 18, wherein
the processor further executes a step of obtaining a result of inspection pertaining to a cognitive ability of the user, and
in the step of evaluating the user, the user is evaluated based on a transition of the cognitive ability of the user that is confirmed from the record of the light treatment and the result of the inspection.

### (Supplement 20)

The method according to supplement 19, wherein
the processor evaluates a transition of a cognitive ability of the user by comparing a value indicated by the result of the inspection with a reference value that is preset.

### (Supplement 21)

A method executed by a treatment light control system 1 including a computer, wherein
a processor of the computer executes:
a step of recording a history of use of visual content as light treatment by a plurality of users;
a step of recording results of inspection of the plurality of users; and
a step of evaluating a plurality of pieces of visual content based on the history of use and the results of inspection.

### (Supplement 22)

The method according to supplement 21, wherein
in the step of evaluating the pieces of visual content,
the processor executes:
a step of extracting a user group that shows a suitable tendency in a transition of a cognitive ability in the results of inspection; and
a step of identifying visual content that is used with high frequency in the extracted user group.

### (Supplement 2-1)

A treatment light control system including:
a light source that applies blinking light;
a controller that controls a blinking frequency of the light source such that the blinking frequency is included in a predetermined frequency band; and
disposition means for disposing a moving liquid in a region to which the light source applies the light.

### (Supplement 2-2)

A treatment light control system that performs treatment for dementia, cranial nerve disease, or mental disorder by inputting a light stimulus with blinking light being visible light through eyes of a user to induce neural oscillation of the user, the treatment light control system including:
a light source that applies, toward the eyes of the user, blinking light that blinks at a frequency in a gamma band of 25 Hz to 100 Hz with such an intensity that the blinking light dazzles the user, the frequency being a frequency of a periodic neuronal activity that relates to a human cognitive function;
a controller that controls a blinking frequency of the light source such that the blinking frequency is included in the gamma band; and
disposition means for disposing a moving liquid in a region to which the light source applies the light, thus scattering the blinking light applied toward the eyes of the user to soften the light stimulus with the blinking light.

### (Supplement 3-1)

A treatment light control system that performs treatment for dementia, cranial nerve disease, or mental disorder by inputting a light stimulus with blinking light being visible light through eyes of a user to induce neural oscillation of the user, the treatment light control system including:
a light source that applies, toward the eyes of the user, blinking light that blinks at a frequency in a gamma band of 25 Hz to 100 Hz with such an intensity that the blinking light dazzles the user, the frequency being a frequency of a periodic neuronal activity that relates to a human cognitive function;
a controller that controls a blinking frequency of the light source such that the blinking frequency is included in the gamma band; and
fine particle discharge means for discharging fine particles that float in a form of fog in a spatial region to which the light source applies the light, thus scattering the blinking light applied toward the eyes of the user to soften the light stimulus with the blinking light.

### (Supplement 3-2)

A treatment light control system that performs treatment for dementia, cranial nerve disease, or mental disorder by inputting a light stimulus with blinking light being visible light through eyes of a user to induce neural oscillation of the user, the treatment light control system including:
a light source that applies, toward the eyes of the user, blinking light that blinks at a frequency in a gamma band of 25 Hz to 100 Hz with such an intensity that the blinking light dazzles the user, the frequency being a frequency of a periodic neuronal activity that relates to a human cognitive function;
a controller that controls a blinking frequency of the light source such that the blinking frequency is included in the gamma band; and
a visual display unit that is disposed in front of the light source, is made of, for example, a liquid crystal panel, an electro-wetting panel, an electrochromic panel, or an organic EL panel having transmittivity, and includes a transmissive panel electrically controlling a transmission pattern of light applied from the light source toward front, the visual display unit displaying a predetermined visual content on a display surface of the transmissive panel while softening a light stimulus caused by the blinking light by controlling a wavelength of the light being transmitted for each of pixels constituting the transmissive panel to switch between transmission and cutting off of the applied blinking light for each pixel.

### (Supplement 3-3)

The treatment light control system according to supplement 3-1, wherein the light source is a part of a visual output device.

### (Supplement 3-4)

The treatment light control system according to supplement 3-1 or 3-2, further including user analysis means for detecting any one of a position, a line of sight, and a hypnagogic state of the user.

### (Supplement 3-5)

The treatment light control system according to supplement 3-1 or 3-2, further including sound stimulus means for outputting a sound stimulus to be input into a user via a sense of hearing, a frequency of alternation of increase and decrease in output intensity of the sound stimulus being included in the gamma band.

### (Supplement 3-6)

The treatment light control system according to supplement 3-1 or 3-2, further including cutaneous stimulus means for outputting an electric or ultrasonic stimulus to be input into a user via a sense of touch, a frequency of alternation of increase and decrease in output intensity of the stimulus being included in the gamma band.

### (Supplement 3-7)

The treatment light control system according to supplement 3-1, wherein
the light source and the fine particle discharge means are both disposed in a bathroom including a bathtub, and
the light source applies the blinking light toward the user in the bathtub.

### (Supplement 3-8)

A method executed by a treatment light control system that includes a computer and performs treatment for dementia, cranial nerve disease, or mental disorder by inputting a light stimulus with blinking light being visible light through eyes of a user to induce neural oscillation of the user, wherein
a processor of the computer executes:
a step of, by a light source, applying, toward the eyes of the user, blinking light that blinks at a frequency in a gamma band of 25 Hz to 100 Hz with such an intensity that the blinking light dazzles the user, the frequency being a frequency of a periodic neuronal activity that relates to a human cognitive function;
a step of, by a controller, controlling a blinking frequency of the light source such that the blinking frequency is included in the gamma band; and
a step of, by fine particle discharge means for discharging fine particles that float in a form of fog in a spatial region to which the light source applies the light, thus scattering the blinking light applied toward the eyes of the user to soften the light stimulus with the blinking light, discharging the fine particles to the spatial region.

### (Supplement 3-9)

A program executed by a treatment light control system that includes a computer and performs treatment for dementia, cranial nerve disease, or mental disorder by inputting a light stimulus with blinking light being visible light through eyes of a user to induce neural oscillation of the user,
the program causing a processor of the computer to execute:
a step of, by a light source, applying, toward the eyes of the user, blinking light that blinks at a frequency in a gamma band of 25 Hz to 100 Hz with such an intensity that the blinking light dazzles the user, the frequency being a frequency of a periodic neuronal activity that relates to a human cognitive function;
a step of, by a controller, controlling a blinking frequency of the light source such that the blinking frequency is included in the gamma band; and
a step of, by fine particle discharge means for discharging fine particles that float in a form of fog in a spatial region to which the light source applies the light, thus scattering the blinking light applied toward the eyes of the user to soften the light stimulus with the blinking light, discharging the fine particles to the spatial region.

### (Supplement 3-10)

A method executed by a treatment light control system that includes a computer and performs treatment for dementia, cranial nerve disease, or mental disorder by inputting a light stimulus with blinking light being visible light through eyes of a user to induce neural oscillation of the user, wherein
a processor of the computer executes:
a step of, by a light source, applying, toward mainly front where the user is positioned and toward the eyes of the user, blinking light that blinks at a frequency in a gamma band of 25 Hz to 100 Hz with such an intensity that the blinking light dazzles the user, the frequency being a frequency of a periodic neuronal activity that relates to a human cognitive function;
a step of, by a controller, controlling a blinking frequency of the light source such that the blinking frequency is included in the gamma band; and
a step of, by a visual display unit that is disposed in front of the light source, is made of, for example, a liquid crystal panel, an electro-wetting panel, an electrochromic panel, or an organic EL panel having transmittivity, and includes a transmissive panel electrically controlling a transmission pattern of light applied from the light source toward front, displaying a predetermined visual content on a display surface of the transmissive panel while softening a light stimulus caused by the blinking light by controlling a wavelength of the light being transmitted for each of pixels constituting the transmissive panel to switch between transmission and cutting off of the applied blinking light for each pixel.

### (Supplement 3-11)

A program executed by a treatment light control system that includes a computer and performs treatment for dementia, cranial nerve disease, or mental disorder by inputting a light stimulus with blinking light being visible light through eyes of a user to induce neural oscillation of the user,
the program causing a processor of the computer to execute:
a step of, by a light source, applying, toward mainly front where the user is positioned and toward the eyes of the user, blinking light that blinks at a frequency in a gamma band of 25 Hz to 100 Hz with such an intensity that the blinking light dazzles the user, the frequency being a frequency of a periodic neuronal activity that relates to a human cognitive function;
a step of, by a controller, controlling a blinking frequency of the light source such that the blinking frequency is included in the gamma band; and
a step of, by a visual display unit that is disposed in front of the light source, is made of, for example, a liquid crystal panel, an electro-wetting panel, an electrochromic panel, or an organic EL panel having transmittivity, and includes a transmissive panel electrically controlling a transmission pattern of light applied from the light source toward front, displaying a predetermined visual content on a display surface of the transmissive panel while softening a light stimulus caused by the blinking light by controlling a wavelength of the light being transmitted for each of pixels constituting the transmissive panel to switch between transmission and cutting off of the applied blinking light for each pixel.

### (Supplement 3-12)

The method according to supplement 3-8 or 3-10, wherein the processor further executes a step of detecting any one of a position, a line of sight, and a hypnagogic state of a user.

### (Supplement 3-13)

The method according to supplement 3-8 or 3-10, wherein the processor further executes, prior to the step of applying the blinking light, a step of turning off an illuminating device other than the light source used in the spatial region.

### (Supplement 3-14)

The method according to supplement 3-8 or 3-10, wherein the processor further executes, prior to the step of applying the blinking light, a step of, by an optical sensor, measuring an amount of light output from an illuminating device other than the light source used in a spatial region that is a space where the user receives the treatment.

### (Supplement 3-15)

A method executed by a treatment light control system that includes a computer and performs treatment for dementia, cranial nerve disease, or mental disorder by inputting a light stimulus with blinking light being visible light through eyes of a user to induce neural oscillation of the user, wherein
a processor of the computer executes:
a step of converting visual content into a visual signal that blinks at a frequency in a gamma band of 25 Hz to 100 Hz, the frequency being a frequency of a periodic neuronal activity that relates to a human cognitive function;
a step of outputting, in a form of a visual content, a blinking visual signal from a visual output device toward the eyes of the user with such an intensity that the visual content dazzles the user; and
a step of, by fine particle discharge means, discharging, in front of the visual output device, fine particles that float in a form of fog, thus scattering the visual signal applied toward the eyes of the user to soften the light stimulus with the visual signal.

### (Supplement 3-16)

The method according to supplement 3-15, wherein
the processor further executes:
a step of converting sound content into a sound signal that alternates increase and decrease in the gamma band; and
a step of outputting the sound signal.

1. A treatment light control system comprising:
a light source configured to apply blinking light;
a controller configured to control a blinking frequency of the light source such that the blinking frequency is included in a predetermined frequency band; and
a fine particle discharge apparatus configured to discharge fine particles into a spatial region to which the light source applies light.

2. A treatment light control system comprising:
a light source configured to apply blinking light toward mainly front where a user is positioned;
a controller configured to control a blinking frequency of the light source such that the blinking frequency is included in a predetermined frequency band; and
a visual display apparatus disposed in front of the light source, configured to display a predetermined visual content on a display surface by transmitting light applied from the light source.

3. The treatment light control system according to claim 1 or 2, wherein
the light source is configured to occupy a certain region orthogonally mainly to a direction of applying the light, and
the blinking frequency of at least a certain region of the light source is less than 100 Hz.

4. The treatment light control system according to claim 1, wherein the light source is a part of a visual output apparatus.

5. The treatment light control system according to claim 1 or 2, further comprising user analysis apparatus configured to detect any one of a position, an attitude, a line of sight, and a hypnagogic state of a user.

6. The treatment light control system according to claim 1 or 2, further comprising sound stimulus apparatus configured to output a sound stimulus to be input into a user via a sense of hearing, a frequency of alternation of increase and decrease in output intensity of the sound stimulus being included in a predetermined frequency band.

7. The treatment light control system according to claim 1 or 2, wherein the treatment light control system is provided and used in a bathroom.

8. A method for operating a treatment light control system comprising circuitry, wherein the circuitry executes:
applying blinking light by a light source;
controlling a blinking frequency of the light source such that the blinking frequency is included in a predetermined frequency band by a controller; and
discharging fine particles into a spatial region to which the light source applies light by a fine particle discharge apparatus.

9. A method for operating a treatment light control system comprising circuitry, wherein the circuitry executes:
applying blinking light toward mainly front where a user is positioned by a light source;
controlling a blinking frequency of the light source such that the blinking frequency is included in a predetermined frequency band by a controller; and
displaying a predetermined visual content on a display surface by transmitting light applied from the light source, a visual display apparatus being disposed in front of the light source by the visual display apparatus.

10. The method according to claim 8 or 9, wherein the circuitry executes detecting any one of a position, an attitude, a line of sight, and a hypnagogic state of a user.

11. The method according to claim 8 or 9, wherein the circuitry executes turning off an illuminating apparatus.

12. A method for operating a treatment light control system comprising circuitry wherein the circuitry executes:
converting visual content into a visual signal that blinks at a predetermined interval;
outputting, in a form of a visual content, the blinking visual signal from a visual output apparatus; and
discharging fine particles in front of the visual output apparatus.

13. The method according to claim 12, wherein the circuitry executes:
converting sound content into a sound signal that is modulated to the predetermined interval; and
outputting the sound signal.

14. A treatment light control system comprising:
a light source configured to apply blinking light;
a controller configured to control a blinking frequency of the light source such that the blinking frequency is included in a predetermined frequency band; and
a disposition apparatus configured to dispose a moving liquid in a region to which the light source applies light.
